(19) United States
(12) Patent Application Publication
(10) Pub. No.: US 2010/0222401 A1
(43) Pub. Date: Sep. 2, 2010

(54) COMPOSITIONS AND METHODS FOR TREATING PATHOLOGIC ANGIOGENESIS AND VASCULAR PERMEABILITY

(75) Inventors: Dean Li, Salt Lake City, UT (US); Christopher Jones, Salt Lake City, UT (US); Nyall London, Bountiful, UT (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: University of Utah Research Foundation, Salt Lake City, UT (US)

(21) Appl. No.: 12/667,168

(22) PCT Filed: Apr. 16, 2009
(86) PCT No.: PCT/US09/40848

§ 371 Date: Feb. 24, 2010

Related U.S. Application Data
(60) Provisional application No. 61/045,580, filed on Apr. 16, 2008.
(60) Provisional application No. 61/073,880, filed on Jun. 19, 2008.

(51) Int. Cl. A61K 31/4196; C07K 14/435; C07D 249/08; C07D 405/02

(52) U.S. Cl. 514/384; 530/350; 548/267.6; 548/266.4

(57) ABSTRACT

Compounds, compositions and methods for inhibiting vascular permeability and pathologic angiogenesis by modulating a signaling pathway delineated herein are described. Moreover, methods for producing and screening compounds and compositions capable of modulating the signaling pathway described herein, inhibiting vascular permeability, and inhibiting pathologic angiogenesis are also provided.

### BACKGROUND OF THE INVENTION

Though the formation of the vertebrate vasculature of any organ system is a complex process that is orchestrated by a constellation of growth factors and guidance cues (Jain et al., 2003), recent studies have increased our understanding of the signaling cascades that regulate angiogenesis. For example, it is increasingly clear that molecular programs, which direct trajectory of axons and the formation of the neural network, have important roles in generating the highly stereotypical pattern of the mature vascular network (Carmeliet et al., 2005; Urness et al., 2004; and Jones et al., 2007).

During the initial phase of vascular development in mammals, which is referred to as vasculogenesis, endothelial cells differentiate, migrate and coalesce to form the central axial vessels, the dorsal aortae and cardinal veins. The second phase, called angiogenesis, is characterized by the sprouting of new vessels from the nascent plexus to form a mature circulatory system. VEGF (or VPF) is critical for both of these first two phases: the differentiation and survival of endothelial cells during vasculogenesis as well as proliferation and permeability during angiogenesis. Following this angiogenic remodeling, the endothelium secretes platelet-derived growth factor (PDGF), which induces the recruitment and differentiation of vascular smooth muscle cells. Subsequently, the vascular smooth muscle cells secrete angiopoietins, which ensure proper interaction between endothelial and vascular smooth muscle cells. Finally, the vascular smooth muscle cells deposit matrix proteins, such as elastin, that inhibit vascular smooth muscle cell proliferation and differentiation, thereby stabilizing the mature vessel. Thus, to establish and maintain a mature vascular network, the endothelial and smooth muscle compartments of a vessel must interact via autocrine and paracrine signaling. The gaps between endothelial cells (cell junctions) forming the vascular endothelium are strictly regulated depending on the type and physiological state of the tissue. For example, in a mature vascular bed, endothelial cells do not behave independently of one another; rather, they form a monolayer that prevents the movement of protein, fluid and cells from the endothelial lumen into the surrounding tissue.

Even after development, the vascular system is continually exposed to events, conditions or pathogens that cause injury, ischemia, and inflammation, which typically result in the release of cytokines and angiogenic factors, such as vascular endothelial growth factor (VEGF). Initially, VEGF was described, purified and cloned as vascular permeability factor (VPF), based on its ability to induce blood vessels to leak. VEGF destabilizes endothelial cell-cell junctions, leading to endothelial permeability, stimulates endothelial proliferation and migration, and promotes vascular sprouting and edema. These functions serve to deconstruct a stable vascular network producing leaky new blood vessels. In many contexts, the release of cytokines and angiogenic factors in response to injury, ischemia and inflammation is desirable, in that such a response leads initiates a restorative or healing processes. However, excessive angiogenesis and vascular leak (e.g., endothelial hyperpermeability) underscore the pathologies of several diseases and pathologic conditions.

For example, in the developed world, pathologic angiogenesis and endothelial hyperpermeability in the retinal or choroidal vascular beds are the most common causes of catastrophic vision loss. New and dysfunctional blood vessels leak, bleed or stimulate fibrosis that in turn precipitates edema, hemorrhage, or retinal detachment compromising vision. The major diseases sharing this pathogenesis include proliferative diabetic retinopathy (DR), non-proliferative diabetic macular edema (DME), and age-related macular degeneration (AMD) (Dorrell et al., 2007; Afzal et al., 2007). Approximately 15 million Americans over the age of 65 suffer from AMD, and 10% of these patients will experience visual loss as a result of choroidal neovascularization. Further, more than 16 million Americans are diabetic, and over 400,000 new patients suffer from retinal edema or neovascularization. Given that the current number of 200 million diabetics worldwide is likely to double in the next 20 years, and that over 8% of such patients suffer from microvascular complications, the number of patients that will experience vision loss from diabetic eye disease is unfortunately set to increase rapidly. Though less prevalent than DR, DME and AMD, retinopathy of prematurity (ROP) and ischemic retinal vein occlusion (IRVO) are also associated with pathologic angiogenesis and endothelial hyperpermeability in the retinal or choroidal vascular beds and lack effective treatment.

In addition to diseases of the eye, pathologic angiogenesis is also associated with tumor formation and growth. Tumor angiogenesis is the proliferation of a network of blood vessels that penetrates into cancerous growths, supplying nutrients and oxygen and removing waste products. With angiogenesis tumor growth proceeds, without it, growth is slowed or stops entirely. Tumor angiogenesis typically starts with cancerous tumor cells releasing molecules that send signals to surrounding normal host tissue, which activates production of proteins that encourage growth of new blood vessels. Angiogenesis is regulated by both activator and inhibitor molecules. Under normal conditions, the inhibitors predominate, blocking growth. However, during tumor formation and growth, tumor cells release angiogenesis activators, causing such activators to increase in number/concentration. Such an increase in angiogenesis activators results in the growth and division of vascular endothelial cells and, ultimately, the formation of new blood vessels.

Several different proteins, as well as several smaller molecules, have been identified as “angiogenic.” Among these molecules, two proteins appear to be the most important for sustaining tumor growth: vascular endothelial growth factor (VEGF) and basic fibroblast growth factor (bFGF). VEGF and bFGF are produced by many kinds of cancer cells and by certain types of normal cells. VEGF and bFGF are first synthesized inside tumor cells and then secreted into the surrounding tissue. When they encounter endothelial cells, they bind to specific proteins, called receptors, sitting on the outer surface of the cells. The binding of either VEGF or bFGF to its appropriate receptor activates a series of relay proteins that transmits a signal into the nucleus of the endothelial cells. The nuclear signal ultimately prompts a group of genes to make products needed for new endothelial cell growth. The activation of endothelial cells by VEGF or bFGF sets in motion a series of steps toward the creation of new blood vessels. First, the activated endothelial cells produce matrix metalloproteinases (MMPs), a special class of degradative enzymes. These enzymes are then released from the endothelial cells into the surrounding tissue. The MMPs break down the extracellular matrix—support material that fills the spaces between cells and is made of proteins and polysaccharides. Breakdown of this matrix permits the migration of endothelial cells. As they migrate into the surrounding tissues, activated endothelial cells begin to divide and organize into hollow tubes that evolve gradually into a mature network of blood vessels.

Additional diseases and disorders characterized by undesirable vascular permeability include, for example, edema associated with brain tumors, ascites associated with malignancies, Meigs' syndrome, lung inflammation, nephrotic syndrome, pericardial effusion, pleural effusion, acute lung injury, inflammatory bowel disease, ischemia/reperfusion injury in stroke, myocardial infarction, and infectious and non-infectious diseases that result in a cytokine storm. Though a cytokine storm is the systemic expression of a healthy and vigorous immune system, it is an exaggerated immune response caused by rapidly proliferating and highly activated T-cells or natural killer (NK) cells and results in the release of more than 150 inflammatory mediators (cytokines, oxygen free radicals, and coagulation factors). Both pro-inflammatory cytokines (such as Tumor Necrosis Factor-alpha, InterLeukin-1, and InterLeukin-6) and anti-inflammatory cytokines (such as interleukin 10, and interleukin 1 receptor antagonist) are elevated in the serum, and it is the fierce and often lethal interplay of these cytokines is referred to as a “cytokine storm.”

Cytokine storms can occur in a number of infectious and non-infectious diseases including, for example, graft versus host disease (GVHD), adult respiratory distress syndrome (ARDS), sepsis, avian influenza, smallpox, and systemic inflammatory response syndrome (SIRS). In the absence of prompt intervention, a cytokine storm can result in permanent lung damage and, in many cases, death. Many patients will develop ARDS, which is characterized by pulmonary edema that is not associated with volume overload or depressed left ventricular function. The end stage symptoms of a disease precipitating the cytokine storm may include one or more of the following: hypotension; tachycardia; dyspnea; fever; ischemia or insufficient tissue perfusion; uncontrollable hemorrhage; severe metabolism dysregulation; and multisystem organ failure. Deaths from infections that precipitate a cytokine storm are often attributable to the symptoms resulting from the cytokine storm and are, therefore, not directly caused by the relevant pathogen. For example, deaths in severe influenza infections, such as by avian influenza or “bird flu,” are typically the result of ARDS, which results from a cytokine storm triggered by the viral infection.

Because of its involvement in angiogenesis and vascular permeability, much attention has been focused on vascular endothelial growth factor (VEGF). Nevertheless, as VEGF is only one of many angiogenic, permeability and inflammatory factors that contribute to angiogenesis and vascular permeability, there is continued value in identifying pathways and developing methods that affect VEGF functionality as well as the functionality of other angiogenic, permeability, or inflammatory factors.

### SUMMARY

A signaling pathway whereby Robo4 signaling can inhibit protrusive events involved in cell migration, stabilize endothelial cell-cell junctions, and block pathological angiogenesis is described herein. As is shown herein, expression of Robo4 confers responsiveness to Slit2, and Slit2-Robo4 signaling negatively regulates cellular protrusive activity stimulated by cell adhesion. Such negative regulation is mediated by interaction of Robo4 with the adaptor protein, paxillin, and its paralogues, which recruits ARF-GAPs such as GIT1, leading to local inactivation of Adp ribosylation factor 6 (ARF6). This signaling pathway thereby interferes with adhesion-mediated Rac1 activation and cell protrusion.

As is further described herein, modulation of ARF GTPase activating proteins (“ARF-GAP” in the singular or “ARF-GAPs” in the plural) and ARF GTP exchange factors (“ARF-GEF” in the singular or “ARF-GEFs” in the plural) can be accomplished without Robo4 signaling, and such modulation can be used to inhibit cellular protrusive activity, vascular leak, endothelial permeability, and/or pathologic angiogenesis. Therefore, multiple targets for modulation of signaling pathways that contribute to inhibition of cellular protrusive activity, vascular leak, endothelial permeability, and/or pathologic angiogenesis are provided herein, including, for example, multiple targets defined within in the presently described Slit2-Robo4 signaling pathway.

Compounds, compositions and methods for inhibiting vascular permeability and pathologic angiogenesis by modulating the singnaling pathway delineated herein are also described. Moreover, methods for producing and screening compounds and compositions capable of modulating the signaling pathway described herein, inhibiting vascular permeability, and inhibiting pathologic angiogenesis are also provided.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions. As it is used herein, the term “Mock” indicates a sham preparation that does not include an active Slit protein.

FIG. 1 shows Robo4-mediated vascular guidance requires the cytoplasmic tail of the receptor. Shown is the results of confocal microscopy of 48 hpf TG(fli:egfp)yl embryos (A) un-injected, (B) injected with robo4 morpholino, (C) robo4 morpholino and wild-type murine robo4 RNA, and (D) robo4 morpholino and robo4Δtail RNA. Quantification is shown in FIG. 7. FIG. 1E shows model of defective vascular guidance in robo4 morphant embryos. 5× and 20× images are shown in the left and right panels, respectively. DLAV=dorsal longitudinal anastomosing vessel. PAV=parachordal vessel. DA=dorsal aorta. PCV=posterior cardinal vein.

FIG. 2 shows Robo4-dependent inhibition of haptotaxis requires the aminoterminal half of the cytoplasmic tail. FIG. 2A shows schematic representation of cDNA constructs used in the haptotaxis migration assays. TM represents the transmembrane domain. CC0 and CC2 are conserved cytoplasmic signaling motifs found in Robo family members. HA=hemagglutinin epitope. FIG. 2B and FIG. 2C show HEK 293 cells were co-transfected with GFP and the indicated constructs and 36 hours later subjected to haptotaxis migration on membranes coated with 5 μg/ml fibronectin and either Mock preparation or Slit2. Expression of Robo4 constructs was verified by Western blotting (Inset). Results are presented as the mean±SE.

FIG. 3 shows Robo4 interacts with Hic-5 and paxillin in HEK 293 cells. FIG. 3A shows HEK 293 cells were co-transfected with the Robo4 cytoplasmic tail-HA and Hic-5-V5, or empty vector (pcDNA3) and Hic-5-V5. Robo4 was immunoprecipitated with HA antibodies and Hic-5 was detected by western blotting with V5 antibodies. FIG. 3B shows total cell lysates from Cho-K1, HEK 293 and NIH 3T3 cells were probed with antibodies to Hic-5 and paxillin. FIG. 3C shows HEK 293 cells were co-transfected with paxillin-V5 and Robo4 cytoplasmic tail-HA or empty vector (pcDNA3). Robo4 was immunoprecipitated from cell lysates with HA antibodies and paxillin was detected by western blotting with V5 antibodies. FIG. 3D shows HEK 293 cells were transfected with full length Robo4-HA and paxillin-V5, and stimulated with Slit2 for 5 minutes. Robo4 was immunoprecipitated from cell lysates with HA antibodies and paxillin was detected by western blotting with V5 antibodies.

FIG. 4 shows paxillin interacts with Robo4 through a novel motif that is required for Robo4-dependent inhibition of haptotaxis. FIG. 4A shows schematic representation of GST-Robo4 fusion proteins used in pull down assays shown in panel B. FIG. 4B shows GST-Robo4 fusion proteins were purified form E. coli and incubated with recombinant purified paxillin. Paxillin was detected by western blotting with paxillin-specific monoclonal antibodies. FIG. 4C shows schematic representation of GST-Robo4 fusion proteins used in pull down assays described in panel D. FIG. 4D shows GST-Robo4 fusion proteins were purified form E. coli and incubated with recombinant purified paxillin. Paxillin was detected by western blotting with paxillin-specific monoclonal antibodies. FIG. 4E shows GST-Robo4 wild-type or GST-Robo4ΔPIM were purified from E. coli and incubated with recombinant purified paxillin or in vitro transcribed/translated Mena-V5. Paxillin and Mena were detected with paxillin-specific monoclonal antibodies and V5 antibodies, respectively. FIG. 4F shows HEK 293 cells were transfected with GFP and the indicated constructs and 36 hours later subjected to haptotaxis migration on membranes coated with 5 μg/ml fibronectin and either Mock preparation or Slit2. Expression of Robo4 constructs was verified by western blotting (Inset). Results are presented as the mean±SE.

FIG. 5 shows Robo4 suppresses cell spreading through inactivation of Rac. FIG. 5A. FIG. 5D, and FIG. 5G show HEK 293 cells were transfected with GFP and the indicated constructs and 36 hours later subjected to cell spreading assays on coverslips coated with 5 μg/ml fibronectin and either Mock preparation or Slit2. Results are presented as the mean±SE. FIG. 5B and FIG. 5E show HEK 293 cells were transfected with the indicated constructs and 36 hours later plated onto dishes coated with 5 μg/ml fibronectin and either Mock preparation or Slit2. Following a 5-minute incubation, cells were lysed and GTP-Rac was precipitated with GST-PBD. Rac was detected by western blotting with a Racspecific monoclonal antibody. FIG. 5H shows HUVEC were incubated for 60 minutes with Slit2, stimulated with 25 ng/ml VEGF for 5 minutes, lysed and GTP-Rac was precipitated with GST-PBD. Rac was detected by western blotting with a Rac-specific monoclonal antibody. Slit2-dependent inhibition of (C) and (F) adhesion induced- and (I) VEGF-induced Rac activation was quantified by densitometry. Results are presented as mean±SE.

FIG. 6 shows a paxillinΔLim4 mutant does not interact with Robo4, or support Slit2-Robo4-mediated inhibition of cell spreading. FIG. 6A shows a schematic representation of paxillin constructs used in panels B, C and D. FIG. 6B shows HEK 293 cells were co-transfected with the Robo4 cytoplasmic tail-HA and paxillin-V5, or empty vector (pcDNA3) and paxillin-V5. Robo4 was immunoprecipitated from cell lysates with HA antibodies, and paxillin was detected by western blotting with V5 antibodies. FIG. 6C shows HEK 293 cells were co-transfected with the Robo4 cytoplasmic tail-HA and either wild-type paxillin-V5 or paxillinΔLim4-V5. Robo4 was immunoprecipitated with HA antibodies, and paxillin was detected by western blotting with V5 antibodies. FIG. 6D shows Endogenous paxillin was knocked down in HEK 293 cells using siRNA and reconstituted with either wild-type chicken paxillin or chicken paxillinΔLim4. Knock down and reconstitution were visualized by western blotting with paxillin antibodies and quantified by densitometry. Paxillin expression was determined to be 35% of wild-type levels. FIG. 6E shows HEK 293 cells subjected to knock down/reconstitution were subjected to spreading assays on coverslips coated with 5 μg/ml fibronectin and either Mock preparation or Slit2. Results are presented as the mean±SE.

FIG. 7 shows the paxillin interaction motif is required for repulsive vascular guidance. FIG. 7A shows Quantification of vascular pattering defects in uninjected (n=66), robo4 morpholino (n=56), robo4 morpholino and wild-type murine robo4 RNA (n=60), robo4 morpholino and robo4Δtail RNA (n=17), and robo4 morpholino and robo4ΔPIM RNA (n=45) injected TG(fli:egfp)yl embryos. Representative images are shown in FIG. 1. FIG. 7B shows a model of a Slit2-Robo4 signaling axis that inhibits cell migration, spreading and Rac activation.

FIG. 8 shows splice-blocking morpholinos suppress expression of robo4 in zebrafish embryos. FIG. 8A shows a schematic representation of the robo4 locus in Danio rerio and the encoded Robo4 protein. The exon targeted with the splice-blocking morpholino is indicated, as is the location of the primers used to amplify robo4 cDNA. FIG. 8B shows RNA from uninjected embryos and embryos injected with robo4 spliceblocking morpholinos was isolated and used to reverse transcribe cDNA. The cDNA was then used to amplify robo4 and the resulting fragments were separated by agarose gel electrophoresis and visualized by ethidium bromide staining.

FIG. 9 shows Hic-5 is a Robo4-interacting protein. FIG. 9A shows a schematic representation of full-length Hic-5 and the cDNA clones recovered from the yeast two-hybrid screen. FIG. 9B shows S. cerevisiae strain PJ694-A was transformed with the indicated plasmids and plated to synthetic media lacking Leucine and Tryptophan, or Leucine, Tryptophan, Histidine and Alanine. Colonies capable of growing on nutrient deficient media were spotted onto the same media, replica plated, and either photographed or used for the beta-galactosidase assay.

FIG. 10 shows the paxillin interaction motif lies between CC0 and CC2 in the Robo4 cytoplasmic tail. Schematic representation of the murine Robo4 protein and identification of the amino acids comprising the paxillin interaction motif.

FIG. 11 shows the Robo4 cytoplasmic tail does not inhibit Cdc42 activation nor interact with srGAP1. FIG. 11A shows HEK 293 cells expressing Robo4 were plated onto bacterial Petri dishes coated with 5 μg/ml fibronectin and either Mock preparation or Slit2. Following a 5-minute incubation, cells were lysed, and GTP-Cdc42 was precipitated with GST-PBD. Cdc42 was detected by western blotting with a Cdc42-specific monoclonal antibody. FIG. 11B shows HEK 293 cells were transfected with the indicated plasmids, and Robo1/Robo4 were immunoprecipitated with HA antibodies. srGAP1 was detected by western blotting with Flag M2 antibodies.

FIG. 12 shows slit reduces retinopathy of prematurity, which is an FDA standard for factors that affect diabetic retinopathy, retinopathy of prematurity, and age related macular degeneration. FIG. 12A shows percent neovascularization of the retina in wildtype mice receiving Mock preparation compared to those receiving Slit protein. There was a 63% reduction in neovascularization in mice treated with Slit treated mice as compared to wildtype mice. N=6, P<0.003. FIG. 12B shows percent neovascularization of the retina in wildtype mice receiving Mock preparation compared to those receiving saline control. N=5, P<0.85. FIG. 12C shows percent neovascularization of the retina in knockout mice compared to slit. N=1.

FIG. 13 shows slit and netrin can reduce VEGF-induced dermal permeability.

FIG. 14 shows slit can reduce VEGF mediated retinal permeability.

FIG. 15 shows semaphorin like VEGF increases dermal permeability.

FIG. 16 shows that Robo4 blocks Rac-dependent protrusive activity through inhibition of ARF6. CHO-K1 cells stably expressing αIIb or αIIb-Robo4 cytoplasmic tail were plated on dishes coated with fibronectin or fibronectin and fibrinogen, lysed and GTP-ARF6 was precipitated with GST-GGA3. ARF6 was detected by western blotting with an ARF6-specific monoclonal antibody (See, FIG. 16A). CHO-K1 cells stably expressing αIIb or αIIb-Robo4 cytoplasmic tail were cotransfected with GFP and either an empty vector or the GIT1-PBS, and subjected to spreading assays on coverslips coated with fibronectin or fibronectin and fibrinogen. The area of GFP-positive cells was determined using ImageJ, with error bars indicating SEM (See, FIG. 16B). HEK 293 cells were co-transfected with GFP and the indicated constructs and 36 h later were subjected to spreading assays on fibronectin and either Mock preparation or a Slit2 protein (See, FIG. 16C). In all panels, error bars indicate mean±SE. Expression of Robo4 and ARNO was verified by western blotting (data not shown). HEK 293 cells were co-transfected with GFP and the indicated constructs and 36 h later were plated on dishes coated with fibronectin and either Mock preparation or a Slit2 protein. GTP-Rac was precipitated with GST-PBD and Rac was detected with a Rac1-specific monoclonal antibody (See, FIG. 16D).

FIG. 17 illustrates the results of immunoprecipitation reactions that demonstrate the Robo4 receptor binds to the Slit ligand. FIG. 17A shows the results of immunoprecipitation of cell lysates from untransfected human embryonic kidney cells (HEK), HEK cells transfected with Slit tagged with a myc epitope (Slit-myc), HEK cells transfected with Robo4 tagged with a HA epitope (Robo4-HA) and HEK cells transfected with a control vector (Control-HEK). Western blot analysis of the Slit-myc cell lysates serves as a control and demonstrates that the Slit protein has a mass of approximately 210 kD, as previously reported (lane 1). Slit-myc protein is also detected by Western blot with an anti-myc antibody after Slit-myc and Robo4-HA cell lysates were combined and immunoprecipitated with an anti-HA antibody (lane 6). The specificity of this interaction is confirmed by the absence of detectable Slit protein with all other combinations of lysates. The same amount of lysate was used in each experiment. The lower bands in lanes 2-6 correspond to immunoglobulin heavy chains. FIG. 17B shows the results of immunoprecipitation of conditioned media from untransfected HEK cells (HEK CM), HEK cells transfected with Slit tagged with a myc epitope (Slit-myc CM), HEK cells transfected with the N-terminal soluble ectodomain of Robo4 tagged with the HA epitope (NRobo4-HA CM) and HEK cells transfected with control vector (Control-HEK CM). The full-length Slit-myc protein (210 KD) and its C-terminal proteolytic fragment (70 KD) are detected in Slit-myc CM by an anti-myc antibody (lane 1). As in FIG. 17A, Slit-myc protein is also detected by Western blot after Slit-myc and Robo4-HA conditioned media are combined and immunoprecipitated with an anti-HA antibody (lane 6). The specificity of this interaction is confirmed by the absence of Slit protein with all other combinations of conditioned media. As shown in FIG. 17C-FIG. 17F, Slit protein binds to the plasma membrane of cells expressing Robo4. Binding of Slit-myc protein was detected using an anti-myc antibody and an Alexa 594 conjugated anti-mouse antibody. Binding is detected on the surface of Robo4-HEK cells (FIG. 17F) but not Control-HEK cells (FIG. 17D).

FIG. 18 illustrates that Robo4 expression is endothelial-specific and stalk-cell centric. FIG. 18A illustrates retinal flatmounts prepared from P5 Robo4^{+/AP} mice and stained for Endomucin (endothelial cells), NG2 (pericytes) and Alkaline Phosphatase (AP; Robo4). The top-most arrow pointing to the right in the upper left panel indicates a tip cell, and the remaining arrows indicate pericytes (NG2-positive). “T” also indicates tip cells. FIG. 18B illustrates retinal flatmounts prepared from adult Robo4^{+/AP} mice and stained for NG2 (pericytes) and AP (Robo4), with the arrows included in FIG. 18B indicating pericytes (NG2-positive). FIG. 18C shows the results of quantitative RT-PCR (qPCR) performed on the indicated samples using primers specific for PECAM, Robo1 and Robo4. As used in FIG. 18C: “HAEC” represents Human Aortic Endothelial Cells; “HMVEC” represents Human Microvascular Endothelial Cells; and “HASMC” represents Human Aortic Smooth Muscle Cells. FIG. 18D illustrates the results of probing total cell lysates from HMVEC and HASMC with antibodies to Robo4, VE-Cadherin, Smooth Muscle Actin and ERK1/2.

FIG. 19 illustrates that Robo4 signaling inhibits VEGF-A-induced migration, tube formation, permeability and Src family kinase (SFK) activation. Lung endothelial cells (ECs) isolated from Robo4^{+/+} and Robo4^{AP/AP} mice were used in endothelial cell migration (FIG. 19A), tube formation (FIG. 19B), in vitro permeability (FIG. 19C), Miles assay (FIG. 19D) and retinal permeability assay (FIG. 19E). Human microvascular endothelial cells were stimulated with VEGF-A in the presence of a Mock preparation or a Slit2 protein for 5 minutes, lysed and subjected to western blotting with phospho-VEGFR2 antibodies (FIG. 19F), western blotting with phospho-Src antibodies (FIG. 19G) and Rac activation assays (FIG. 19H). In all panels, * represents p<0.05, ** represents p<0.005, *** represents p<0.0005, NS indicates “not significant” and error bars represent SEM.

FIG. 20 illustrates that Robo4 signaling inhibits pathologic angiogenesis in an animal model of oxygen-induced retinopathy (“OIR”) and in an animal model of choroidal neovascularization (“CNV”). Neonatal Robo4^{+/+} and Robo4^{AP/AP} mice were subjected to oxygen-induced retinopathy and perfused with fluorescein isothiocyanate (FITC)-dextran (green). Retinal flatmounts were prepared for each condition and analyzed by fluorescence microscopy. Arrows indicate areas of pathological angiogenesis (FIG. 20A through FIG. 20D). Quantification of pathologic angiogenesis observed in FIG. 20A through FIG. 20D is provided in FIG. 20 E. In the CNV model, 2-3 month old Robo4^{+/+} and Robo4^{AP/AP} mice were subjected to laser-induced choroidal neovascularization. Choroidal flatmounts were prepared, stained with isolectin and analyzed by confocal microscopy (FIG. 20F through FIG. 20I. Quantification of pathologic angiogenesis observed in FIG. 20F through FIG. 20I is provided in FIG. 20J. In all panels, * represents p<0.05, ** represents p<0.005, *** represents p<0.0005, NS indicates “not significant” and error bars represent SEM.

FIG. 21 illustrates that Robo4 signaling inhibits bFGF-induced angiogenesis and thrombin-stimulated endothelial hyperpermeability. In carrying out the experiments that provided the results illustrated in FIG. 21A murine lung endothelial cells were subjected to tube formation assays on matrigel in the presence of bFGF and Mock preparation or a Slit2 protein. In carrying out the experiments that provided the results illustrated in FIG. 21B, murine lung endothelial cells were subjected to thrombin-induced permeability assays on fibronectin-coated Transwells.

FIG. 22 illustrates that Robo4 signaling reduces injury and inflammation in a model of acute lung injury. Mice were exposed to intratracheal LPS and treated with either Slit protein or a Mock preparation. The concentrations of inflammatory cells and protein in bronchoalveolar lavages (BAL) were significantly reduced by treatment with Slit protein.

FIG. 23 illustrates different constructs for Slit proteins and shows that recombinant Slit peptides as small as Slit2-D1 (40 kD) are active. In FIG. 23A, different constructs for the Slit protein are depicted. The four leucine rich domains (LRR), the epidermal growth factor homology region (EGF) and the c-terminal tags (MYC/HIS) are indicated. Inhibition of VEGF mediated endothelial cell migration by the different Slit constructs (2 nM) is shown in FIG. 23B.

FIG. 24 shows the effect of administering a Slit2 protein on the survival of mice infected with Avian Flu Virus in accordance with a mouse model of avian flu.

FIG. 25 illustrates the genomic traits of knockout mice described in Example 14.

FIG. 26 illustrates that the Robo4 cytoplasmic tail suppresses fibronectin-induced protrusive activity. FIG. 26A is a schematic representation of cDNA constructs used in the migration and spreading assays. TM=transmembrane domain. CC0 and CC2 are conserved cytoplasmic signaling motifs found in Robo family members. FIG. 26B, HEK 293 cells were co-transfected with GFP and the indicated constructs and 36 h later subjected to spreading assays on coverslips coated with 5 μg/ml fibronectin and either mock or Slit2. The area of GFP-positive cells was determined using ImageJ. Mock indicates a sham preparation of Slit2. Expression of Robo4 constructs was verified by Western blotting (data not shown). FIG. 26C, CHO-K1 cells stably expressing αIIb or αIIb-Robo4 cytoplasmic tail were subjected to spreading assays on coverslips coated with fibronectin or fibronectin and fibrinogen. Cell area was determined using ImageJ.

FIG. 27 shows the results of an immunoprecipitation experiment, wherein CHO-K1 cells were transfected with the indicated constructs and 36 h later plated onto dishes coated with 5 μg/ml fibronectin or 5 μg/ml fibronectin/fibrinogen. Following a 5-min incubation, cells were lysed and GTP-Rac was precipitated with GST-PBD. Rac was detected by western blotting with a Rae-specific monoclonal antibody.

FIG. 28 illustrates that Slit2 inhibits endothelial cell protrusion via GIT1. FIG. 28A, ECs were subjected to haptotaxis migration assays on membranes coated with 5 μg/ml fibronectin and either mock or Slit2. Cells on the underside of the filter were enumerated and migration on fibronectin/mock membranes was set at 100%. FIG. 28B ECs were subjected to spreading assays on fibronectin and either mock or Slit2. Cell area was determined using ImageJ. FIG. 28C. ECs were plated on dishes coated with fibronectin and either mock or Slit2, lysed and GTP-ARF6 was precipitated with GST-GGA3. FIG. 28D, ECs were plated on dishes coated with VEGF-165 and either mock or Slit2, lysed and GTP-ARF6 was precipitated with GST-GGA3. ARF6 was detected by western blotting with an ARF6-specific monoclonal antibody. **p<0.005. Error bars indicate SEM. Mock indicates a sham preparation of Slit2.

FIG. 29 depicts a chemical structure for Secin-H3.

FIG. 30 illustrates that ARF6 inhibition prevents neovascular tuft formation and endothelial hyperpermeability. DMSO or Secin-H3 were injected into contralateral eyes of wild-type mice and subjected to oxygen-induced retinopathy, laser-induced choroidal neovascularization and VEGF-165-induced retinal hyperpermeability. In FIG. 30A, retinal flatmounts were prepared from neonatal mice subjected to OIR, stained with fluorescent isolectin and analyzed by fluorescence microscopy. Top panels are low magnification images and bottom panels are high magnification images (pathologic neovascular tufts are indicated by yellow and white arrows, respectively). FIG. 30B depicts a quantification of pathologic neovascularization shown in FIG. 30A. In FIG. 30C, choroidal flatmounts were prepared from 2-3 month old mice subjected to laser-induced choroidal neovascularization, stained with fluorescent isolectin and analyzed by confocal microscopy. FIG. 30D shows a quantification of pathologic angiogenesis observed in FIG. 30C. FIG. 30E is a quantification of retinal permeability following intravitreal injection of VEGF-165. Vehicle is DMSO. *p<0.05. Error bars indicate SEM.

FIG. 31 illustrates that the small molecule Secin-H3 inhibits VEGF induced ARF6 GTP.

FIG. 32 illustrates that Secin-H3 inhibits VEGF induced migration of HMVECs.

FIG. 33 illustrates that Src kinase activation (phosphorylation) is not dependent on ARF6.

FIG. 34 illustrates that GIT1 RNAi increases VEGF induced HMVEC permeability.

FIG. 35 is a schematic diagram of pathways described herein.

FIG. 36 illustrates that SecinH3 blocks Arf6 activation and inhibits pathologic angiogenesis and endothelial hyperpermeability in animal models of vascular eye disease. ECs were pre-treated with SecinH3 or DMSO and subjected to Arf6 activation FIG. 36A) and cell migration assays (FIG. 36B). SecinH3 or DMSO were injected into contralateral eyes of wild-type mice and subjected to oxygen-induced retinopathy (FIG. 36C), laser-induced choroidal neovascularization (FIG. 36E) and VEGF-165-induced retinal hyperpermeability (FIG. 36G). Retinal flatmounts were prepared from neonatal mice subjected to OIR (FIG. 36 C), stained with fluorescent isolectin and analyzed by fluorescence microscopy. Top panels are low magnification images and bottom panels are high magnification images of area outlined by white boxes that emphasize the pathologic neovascular tufts (FIG. 36D). Quantification of pathologic neovascularization shown in FIG. 36C. Choroidal flatmounts were prepared from 2-3 month old mice subjected to laser-induced choroidal neovascularization, stained with fluorescent isolectin and analyzed by confocal microscopy (FIG. 36 E). Quantification of pathologic angiogenesis observed in FIG. 36E (FIG. 36F). Quantification of retinal permeability following intravitreal injection of VEGF-165 (FIG. 36G). Vehicle is DMSO. *p<0.05. Error bars indicate s.e.m.

FIG. 37 Provides images illustrating that Slit2 blocks recruitment of paxillin to focal adhesions and Slit2 recruits paxillin to the cell surface.

FIG. 38 Illustrates that Slit2 inhibits endothelial cell protrusion via ArfGAPs. Endothelial cells (ECs) were subjected to haptotaxis migration assays on membranes coated with 5 μg/ml fibronectin and either mock or Slit2 (FIG. 38A). Cells on the underside of the filter were enumerated and migration on fibronectin/mock membranes was set at 100%. ECs were subjected to spreading assays on fibronectin and either mock or Slit2 (FIG. 38B). Cell area was determined using ImageJ. ECs were plated on dishes coated with fibronectin and either mock or Slit2, lysed and GTP-Arf6 was precipitated with GST-GGA3 (FIG. 38C). ECs were plated on dishes coated with VEGF-165 and either mock or Slit2, lysed and GTP-Arf6 was precipitated with GST-GGA3 (FIG. 38D). Arf6 was detected by western blotting with an Arf6-specific monoclonal antibody. Mock indicates a sham preparation of Slit2. **p<0.005. Error bars indicate s.e.m.

FIG. 39 Illustrates that Rho activation was unaltered by Slit2, but Cdc42 activation was significantly reduced by Slit2.

### DETAILED DESCRIPTION OF THE INVENTION

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a polypeptide is disclosed and discussed and a number of modifications that can be made to a number of molecules including the polypeptide are discussed, each and every combination and permutation of polypeptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the meanings that would be commonly understood by one of skill in the art in the context of the present specification.

It must be noted that as used herein and in the appended claims, the singular forms “a,” “an,” and “the” include plural reference unless the context clearly dictates otherwise. Thus, for example, reference to “a polypeptide” includes a plurality of such polypeptides, reference to “the polypeptide” is a reference to one or more polypeptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the term “subject” refers to an animal or human, preferably a mammal, subject in need of treatment for a given disease, condition, event or injury. Thus, the subject can be a human. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between the specifically recited percentages, as compared to native or control levels.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the increase in an activity, response, condition, disease, or other biological parameter can be a 10, 20, 30, 40, 50, 60, 70, 80, 90, 100%, or more, including any amount of increase in between the specifically recited percentages, as compared to native or control levels.

The term “therapeutically effective” means that the amount of the composition used is of sufficient quantity to ameliorate one or more causes or symptoms of a disease or disorder. Such amelioration only requires a reduction or alteration, not necessarily elimination.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

“Alkyl” refers to an optionally substituted hydrocarbon group joined by single carbon-carbon bonds and having 1 to 8 carbon atoms joined together. The alkyl hydrocarbon group may be straight-chain or contain one or more branches. These groups include methyl, ethyl, propyl, isopropyl, butyl, isobutyl, tertiary butyl, pentyl, hexyl, and the like. “Lower alkyl” refers to optionally substituted branched- or straight-chain alkyl having 1 to 4 carbons.

“Alkenyl” refers to an optionally substituted hydrocarbon group containing at least one carbon-carbon double bond between the carbon atoms and containing 2-8 carbon atoms joined together. The alkenyl hydrocarbon group may be branched or straight-chain.

“Cycloalkyl” refers to an optionally substituted cyclic alkyl or an optionally substituted non-aromatic cyclic alkenyl and includes monocyclic and multiple fused ring structures such as bicyclic and tricyclic. The cycloalkyl may have, for example, 3 to 15 carbon atoms. In one embodiment, cycloalkyl has 5 to 12 carbon atoms. Examples of suitable cycloalkyl groups include cyclopropyl, cyclobutyl, cyclopentyl, cyclohexyl and the like.

“Heterocycle” refers to optionally substituted saturated or partially saturated non-aromatic ringed moieties including at least one non-carbon atom. Heterocyclic moieties typically comprise a single ring or multiple fused ring structures, such as bicyclic and tricyclic. In one embodiment, the ring(s) is 5 to 6-membered and typically contains 1 to 3 non-carbon atoms. Non-carbon atoms for heterocycle may be independently selected from nitrogen, oxygen and sulfur.

“Aryl” refers to an optionally substituted aromatic group with at least one ring having a conjugated pi-electron ring system, and includes monocyclic and multiple fused ring structures such as bicyclic and tricyclic. Aryl includes optionally substituted carbocyclic aryl. Examples of suitable aryl groups include phenyl, naphthyl, anthracenyl, phenanthrenyl and the like.

“Heterocyclic aryl” refers to an optionally substituted aromatic group with at least one ring having a conjugated pi-electron ring system including at least one non-carbon atom. Heterocyclic aryl moieties typically comprise one ring or multiple fused ring structures, such as bicyclic and tricyclic. Examples of suitable heterocyclic aryl groups include furanyl, thienyl, pyrrolyl, imidazolyl, pyridinyl, and the like.

“Alkoxy” refers to oxygen joined to an alkyl group. “Lower alkoxy” refers to oxygen joined to a lower alkyl group. In one embodiment, the oxygen is joined to an unsubstituted alkyl 1 to 4 carbons in length. For example, the alkoxy may be methoxy, ethoxy and the like.

“Alkylene” refers to an optionally substituted hydrocarbon chain containing only carbon-carbon single bonds between the carbon atoms. The alkylene chain has 1 to 6 carbons and is attached at two locations to other functional groups or structural moieties. Examples of suitable alkylene groups include methylene, ethylene and the like.

When referring to an active agent “biologically active” and “desired biological activity” refer to an ability to modulate the activity or activation of a targeted molecule. In particular, embodiments, when used in conjunction with an the biologically active agents described herein, “biologically active” and “desired biological activity” refer to an ability to directly or indirectly inhibit or block the activity or activation of a targeted molecule.

As used herein, “small molecule” refers to low molecular weight compounds. For example, in particular embodiments, such small molecule compounds are compounds the exhibit a molecular weight of between 50 daltons to 800 daltons. In alternative embodiments, a small molecule as described herein exhibit a molecular weight selected from the ranges of between 100 daltons and 500 daltons and between 250 daltons to 475 daltons.

As used herein, the terms “treat,” “treating,” and “treatment” refer to a therapeutic benefit, whereby the detrimental effect(s) or progress of a particular disease, condition, event or injury is prevented, reduced, halted or slowed.

A “therapeutically effective amount” is the amount of compound which achieves a therapeutic benefit, such as, for example, retarding a disease in a subject having a disease or prophylactically retarding or preventing the onset of a disease. A therapeutically effective amount may be an amount which relieves to some extent one or more symptoms of a disease or disorder in a subject; returns to normal either partially or completely one or more physiological or biochemical parameters associated with or causative of the disease or disorder; and/or reduces the likelihood of the onset of the disease of disorder.

The terms “pathologic” or “pathologic conditions” refer to any deviation from a healthy, normal, or efficient condition which may be the result of a disease, condition, event or injury.

The term “regulatory sequences” refers to those sequences normally within 100-1000 kilobases (kb) of the coding region of a locus, but they may also be more distant from the coding region, which affect the expression of the gene. Such regulation of expression comprises transcription of the gene, and translation, splicing, and stability of the messenger RNA.

The term “operably linked” refers to a juxtaposition wherein the components so described are in a relationship permitting them to function in their intended manner. For instance, a promoter is operably linked to a coding sequence if the promoter affects its transcription or expression. The term “operably linked” may refer to functional linkage between a nucleic acid expression control sequence (e.g., a promoter, enhancer, or array of transcription factor binding sites) and a second nucleic acid sequence, wherein the expression control sequence directs transcription of the nucleic acid corresponding to the second sequence.

“Isolated,” when used to describe biomolecules disclosed herein, means, e.g., a peptide, protein, or nucleic acid that has been identified and separated and/or recovered from a component of its natural environment. Contaminant components of its natural environment are materials that would typically interfere with diagnostic or therapeutic uses for the isolated molecule(s), and may include enzymes, hormones, and other proteinaceous or non-proteinaceous materials. Methods for isolation and purification of biomolecules described herein are known and available in the art, and one of ordinary skill in the art can determine suitable isolation and purification methods in light of the material to be isolated or purified. Though isolated biomolecules will typically be prepared using at least one purification step, as it is used herein, “isolated” additionally refers to, for example, peptide, protein, antibody, or nucleic acid materials in-situ within recombinant cells, even if expressed in a homologous cell type.

Further, where the terms “isolated”, “substantially pure”, and “substantially homogeneous” are used to describe a monomeric protein they are used interchangeably herein. A monomeric protein is substantially pure when at least about 60 to 75% of a sample exhibits a single polypeptide sequence. A substantially pure protein can typically comprise about 60 to 90% W/W of a protein sample, and where desired, a substantially pure protein can be greater than about 90%, about 95%, or about 99% pure. Protein purity or homogeneity can be indicated by a number of means well known in the art, such as polyacrylamide gel electrophoresis of a protein sample, followed by visualizing a single polypeptide band upon staining the gel. For certain purposes, higher resolution can be provided by using HPLC or other means well known in the art which are utilized for purification.

Throughout the description and claims of this specification, the word “comprise” and variations of the word, such as “comprising” and “comprises,” means “including but not limited to,” and is not intended to exclude, for example, other additives, components, integers or steps.

### Proteins & Peptides

As the terms are used herein, “protein” and “peptide” are simply refer to polypeptide molecules generally and are not used to refer to polypeptide molecules of any specific size, length or molecular weight. Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Table 1 and are referred to as conservative substitutions.

TABLE 1
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the proteins and peptides disclosed herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### Vascular Permeability

As used herein, “vascular permeability” refers to the capacity of small molecules (e.g., ions, water, nutrients), large molecules (e.g., proteins and nucleic acids) or even whole cells (lymphocytes on their way to the site of inflammation) to pass through a blood vessel wall.

Diseases and disorders characterized by undesirable vascular permeability include, for example, edema associated with brain tumors, ascites associated with malignancies, Meigs' syndrome, lung inflammation, nephrotic syndrome, pericardial effusion and pleural effusion. Thus, provided is a method of treating or preventing these or any other disease associated with an increase in vascular permeability or edema. For example, inhibiting edema formation should be beneficial to overall patient outcome in situations such as inflammation, allergic diseases, cancer, cerebral stroke, myocardial infarction, pulmonary and cardiac insufficiency, renal failure, and retinopathies, to name a few. Furthermore, as edema is a general consequence of tissue hypoxia, it can also be concluded that inhibition of vascular leakage represents a potential approach to the treatment of tissue hypoxia. For example, interruption of blood flow by pathologic conditions (such as thrombus formation) or medical intervention (such as cardioplegia, organ transplantation, and angioplasty) could be treated both acutely and prophylactically using inhibitors of vascular leakage.

Ischemia/reperfusion injury following stroke and myocardial infarction is also characterized by vascular permeability and edema. A deficit in tissue perfusion leads to persistent post-ischemic vasogenic edema, which develops as a result of increased vascular permeability. Tissue perfusion is a measure of oxygenated blood reaching the given tissue due to the patency of an artery and the flow of blood in an artery. Tissue vascularization may be disrupted due to blockage, or alternatively, it may result from the loss of blood flow resulting from blood vessel leakage or hemorrhage upstream of the affected site. The deficit in tissue perfusion during acute myocardial infarction, cerebral stroke, surgical revascularization procedures, and other conditions in which tissue vascularization has been disrupted, is a crucial factor in outcome of the patient's condition. Edema can cause various types of damage including vessel collapse and impaired electrical function, particularly in the heart. Subsequent reperfusion, however, can also cause similar damage in some patients, leading to a treatment paradox. While it is necessary, to unblock an occluded blood vessel or to repair or replace a damaged blood vessel, the ensuing reperfusion can, in some cases, lead to further damage. Likewise, during bypass surgery, it is necessary to stop the heart from beating and to have the patient hooked to a heart pump. Some patients who undergo bypass surgery, for example, may actually experience a worsening of condition (“post-pump syndrome”), which may be the result of ischemia during cessation of cardiac function during surgery. An arterial blockage may cause a reduction in the flow of blood, but even after the blockage is removed and the artery is opened, if tissue reperfusion fails to occur, further tissue damage may result. For example, disruption of a clot may trigger a chain of events leading to loss of tissue perfusion, rather than a gain of perfusion.

Additional diseases and disorders characterized by undesirable vascular permeability include, for example, infectious and non-infectious diseases that may result in a cytokine storm. A cytokine storm can be precipitated by a number of infectious and non-infectious diseases including, for example, graft versus host disease (GVHD), adult respiratory distress syndrome (ARDS), sepsis, avian influenza, smallpox, and systemic inflammatory response syndrome (SIRS).

### Pathologic Angiogenesis

Angiogenesis and angiogenesis related diseases are closely affected by cellular proliferation. As used herein, the term “angiogenesis” means the generation of new blood vessels into a tissue or organ. Under normal physiological conditions, humans or animals undergo angiogenesis only in very specific restricted situations. For example, angiogenesis is normally observed in wound healing, fetal and embryonal development and formation of the corpus luteum, endometrium and placenta. The term “endothelium” is defined herein as a thin layer of flat cells that lines serous cavities, lymph vessels, and blood vessels. These cells are defined herein as “endothelial cells.” The term “endothelial inhibiting activity” means the capability of a molecule to inhibit angiogenesis in general. The inhibition of endothelial cell proliferation also results in an inhibition of angiogenesis.

Both controlled and uncontrolled angiogenesis are thought to proceed in a similar manner. Endothelial cells and pericytes are surrounded by a basement membrane and form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

New blood vessels may also form in part by vasculogenesis. Vasculogenesis is distinguished from angiogenesis by the source of the endothelial cells. Vasculogenesis involves the recruitment of endothelial progenitor cells known as angioblasts. These angioblasts can come from the circulation or from the tissue. Vasculogenesis is regulated by similar signaling pathways as angiogenesis. Thus, the term “angiogenesis” is used herein interchangeably with vasculogenesis such that a method of modulating angiogenesis can also modulate vasculogenesis.

Pathologic angiogenesis, which may be characterized as persistent, dysregulated or unregulated angiogenesis, occurs in a multiplicity of disease states, tumor metastasis and abnormal growth by endothelial cells and supports the pathological damage seen in these conditions. The diverse disease states in which pathologic angiogenesis is present have been grouped together as angiogenic-dependent, angiogenic-associated, or angiogenic-related diseases. These diseases are a result of abnormal or undesirable cell proliferation, particularly endothelial cell proliferation.

Diseases and processes mediated by abnormal or undesirable endothelial cell proliferation, including, but not limited to, hemangioma, solid tumors, leukemia, metastasis, telangiectasia psoriasis scleroderma, pyogenic granuloma, myocardial angiogenesis, plaque neovascularization, coronary collaterals, ischemic limb angiogenesis, corneal diseases, rubeosis, neovascular glaucoma, diabetic retinopathy (DR), retrolental fibroplasia, non-proliferative diabetic macular edema (DME), arthritis, diabetic neovascularization, age-related macular degeneration (AMD), retinopathy of prematurity (ROP), ischemic retinal vein occlusion (IRVO), wound healing, peptic ulcer, fractures, keloids, vasculogenesis, hematopoiesis, ovulation, menstruation, and placentation.

### Robo4 Signaling Pathway

The Robo family of proteins is a family of transmembrane proteins known to interact with Slit proteins to guide axonal pathfinding in the nervous system. Robos have been identified in vertebrates, and Robo1-3 are predominantly expressed in the nervous system (Marillat et al., 2002). In contrast, Robo4, also known as Magic Roundabout, is exclusively or predominantly expressed in the vasculature (See, e.g., Park et al., 2003; Huminiecki et al., 2002; and Huminiecki et al., 2002; Seth et al., 2005). Robo4 is further distinguished from Robo1-3 by its divergent sequence: the ectodomain of the neuronal Robos contains five immunoglobulin (Ig) domains and three fibronectin type III (FNIII) repeats, while Robo4 contains two Ig domains and two FNIII repeats (Huminiecki et al., 2002; Park et al., 2003). In addition, Robo1-3 possess four conserved cytoplasmic (CC) motifs, CC0, CC1, CC2 and CC3 (Kidd et al., 1998; Zallen et al., 1998), of which, only CC0 and CC2 are present' in Robo4 (Huminiecki et al., 2002; Park et al., 2003).

Published reports have proposed that Robos can promote angiogenesis in both Slit-dependent and Slit-independent ways. For example, it was reported that Slit2 stimulation of Robo1 induced migration and tube formation in vitro, and promoted tumor angiogenesis in vivo (Feng et al., 2004). Moreover, a study conducted in 2004 showed blocking Robo4 activity with a soluble Robo4 ectodomain inhibited migration and tube formation in vitro, consistent with a positive role for Robo4 during angiogenesis. Further, this study reported that Slit proteins do not bind to Robo4, thereby implicating an unknown ligand for the receptor (Suchting et al., 2004). The notion that Robo4 is proangiogenic has also emerged from recent data showing that overexpression of Robo4 augments endothelial cell adhesion and migration independently of Slit (Kaur et al., 2006).

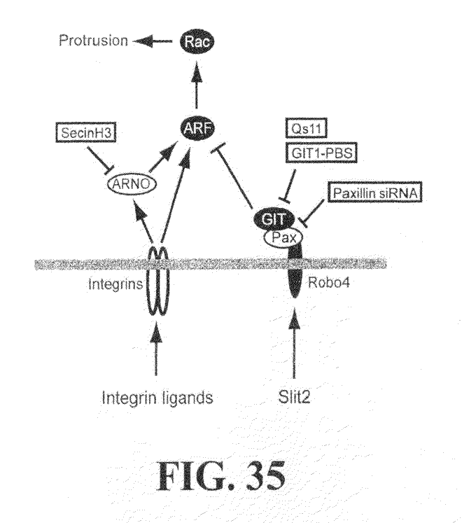

A signaling pathway whereby Robo4 signaling inhibits protrusive events involved in cell migration, stabilize endothelial cell-cell junctions, and block pathological angiogenesis is described herein. The signaling pathway described herein is illustrated in FIG. 35 and provides multiple targets that may be modulated in a manner that affects, for example, cell motility, vascular permeability, and angiogenisis. “Modulation” as used herein includes changing the activity of a target, and “manipulation” as used herein includes a change in the cellular state. As disclosed herein, initiation of Robo4 signaling by ligands of Robo4, such as a Slit-2 Protein as disclosed herein, negatively regulates cell motility and inhibits vascular permeability. In particular, Slit2 elicits a repulsive cue in the endothelium via activation of Robo4, defining a novel signal transduction cascade responsible for such activity. As described herein, Slit2 activation of Robo4, among other things, inhibits Rac activation and, hence, Rac initiated or mediated cell motility and cell spreading.

The teachings provided herein establish a Slit2-dependent association between Robo4 and the adaptor protein paxillin, with the experimental data detailed herein providing biochemical and cell biological evidence that this interaction facilitates Robo4-dependent inhibition of cell migration, cell spreading, and Rac activation. In particular, Robo4 activation initiates paxillin activation of GIT1 and, in turn, GIT1 inhibition of ARF6. Robo4 activation preserves endothelial barrier function, blocks VEGF signaling downstream of the VEGF receptor, and inhibits cellular protrusive activity, vascular leak, endothelial permeability, and/or pathologic angiogenesis. Robo4 activation not only blocks VEGF signaling, but inhibits signaling from multiple angiogenic, permeability and inflammatory factors, including thrombin and bFGF.

As is further disclosed herein, modulation of ARF-GAP activity can be targeted to inhibit cellular protrusive activity, vascular leak, endothelial permeability, and/or pathologic angiogenesis. In particular, activation of ARF-GAPs can inhibit activation of ARF6, and inhibition of ARF6 activity is shown to inhibit cellular protrusive activity, vascular leak, endothelial permeability, and pathologic angiogenesis. In addition, the examples provided herein illustrate that inactivation of ARF-GAPs, such as the ARF-GAP GIT1, can reverse the stabilizing effect of Robo4 signaling on endothelial integrity. Activation of the ARF-GAP GIT1 inhibits activation of ARF6, resulting in an inhibition of VEGF-induced endothelial cell responses. As such, the direct or indirect modulation of ARF6 activity represents a target for controlling vascular permeability and angiogenesis.

In addition to ARF-GAPs, modulation of one or more ARF-GEFs, such as one or more cytohesins, including the ARNO family of cytohesins, can be targeted to inhibit cellular protrusive activity, vascular leak, endothelial permeability, and/or pathologic angiogenesis. Without being bound by a particular theory, it is thought that an effect of Slit2-Robo4 signaling is inhibition or prevention of GTP loading of one or more ARFs. In particular, again without being bound by a particular theory, it is believed that an effect of Slit2-Robo4 signaling is inhibition or prevention of GTP loading of ARF6 and/or ARF1. ARF-GEFs, including those disclosed herein, facilitate GTP loading of ARF6 and inhibition of ARF-GEF activity inhibits ARF activation or activity. As described herein, inhibitors of ARF-GEFs, such as inhibitors of cytohesins, including ARNO and the ARNO family of cytohesins, ARNO results in inhibition of ARF activity as well as inhibition of cellular protrusive activity, vascular leak, endothelial permeability, and/or pathologic angiogenesis.

### Compositions

Compositions for inhibiting vascular permeability and pathologic angiogenesis are provided herein.

Compositions for modulating activity of ARFs are provided herein. In one embodiment, the composition inhibits, either directly or indirectly the activity or activation of an ARF selected from one or both of ARF6 and ARF1. In one such embodiment, the composition includes one or more active agents that directly inhibits an ARF selected from one or both of AFR6 and ARF1. In such an embodiment, the one or more active agents may include one or more ligand of ARF6 and/or ARF1. In another such embodiment, the one or more active agents are selected from one or more small molecules, proteins, peptides or nucleic acids that directly inhibits activity or activation of ARF6 and/or ARF1. In another embodiment, the composition includes one or more active agents that indirectly inhibits an ARF selected from one or both of AFR6 and ARF1. In such an embodiment, the one or more active agents may include an upstream modulator of ARF6 or ARF1 activity or activation, wherein such upstream modulator is selected from one or more small molecules, proteins, peptides or nucleic acids that directly inhibits activity or activation of an upstream modulator of ARF6 or ARF1 activity, such as, for example, the Robo4 receptor, an ARF-GAP, such as GIT1 or an ARF-GEF, such as ARNO or other cytohesin. In another embodiment, a composition as described herein includes one or more active agents that inhibit ARF6 activation of Rac. In one such embodiment, the one or more active agents may be selected from one or more small molecules, proteins, peptides or nucleic acids that act directly or indirectly on or through ARF6 as described herein to inhibit Rac activation by VEGF.

In one embodiment, a composition for modulating the activity of one or more ARFs may include an active agent that indirectly inhibits ARF6 activity or activation by modulating activation, activity or availability of an accessory protein required for ARF6 activity or activation. In one such embodiment, a composition as described herein may include one or more active agents that directly or indirectly inhibit one or more ARF-GEFs, such as, for example, a cytohesin or a member of the ARNO family of cytohesins, such that the activity or activation of one or more ARF family proteins, such as ARF6 and/or ARF1, is reduced. In one such embodiment, a composition as described herein may include one or more active agents that bind ARNO and decrease the activity of individual ARNO proteins such that fewer ARF6 and/or ARF1 proteins are in a GTP-bound state, thereby reducing the pool of active ARF6 proteins. In one embodiment of a composition that includes one or more active agents that directly or indirectly inhibit one or more ARF-GEFs, the one or more active agents may be a ligand of a targeted ARF-GEF. In another such embodiment of a composition that includes one or more active agents that directly or indirectly inhibit one or more ARF-GEFs, the one or more active agents may be selected from one or more small molecules, proteins, peptides or nucleic acids that directly inhibits activity, activation, or availability of the targeted ARF-GEF. In another embodiment of a composition that includes one or more active agents that directly or indirectly inhibit one or more ARF-GEFs, the one or more active agents may be any agent that operates by any mechanism to inhibit the availability, activation or activity of one or more ARF-GEFs.

In another embodiment, a composition for modulating the activity of one or more ARFs may include one or more active agents that increase the activity, activation, or availability of one or more ARF-GAPs, such that the activity or activation of one or more ARF proteins, such as ARF6 and/or ARF1 is reduced. For example, a composition as described herein may include one or more active agents that directly or indirectly increase the activity, activation, or availability of GIT1 such that fewer ARF proteins, for example ARF6 and/or ARF1, are activated, thereby reducing a signal cascade acting through or propagated by ARF6. In one such embodiment, the one or more active agents may include a ligand of GIT1 that binds directly to GIT1 and increases the activation or activity of GIT1 such that the activity or activation of one or more ARF proteins, such as ARF6 and/or ARF1 is reduced. Where a composition includes one or more active agents that directly increases the activity, activation or availability activity of one or more ARF-GAPs, the one or more active agents may be include one or more small molecules, proteins, peptides or nucleic acids that directly or indirectly increase activity, activation or availability of the targeted ARF-GAP. Alternatively, in another embodiment of a composition that includes one or more active agents that increase the activity, activation, or availability of one or more ARF-GAPs, the one or more active agents may be any agent that operates by any mechanism to promote the availability, activation or activity of one or more ARF-GAPs.

In one embodiment, the composition provided herein comprises a ligand of a Robo4 receptor. In one such embodiment, the ligand of Robo4 can be any composition or molecule that can act through Robo4 to negatively regulate cell motility. In another such embodiment, the ligand of Robo4 can be any composition or molecule that can act through Robo4 to inhibit vascular permeability. In yet another such embodiment, the ligand of Robo4 can be any composition or molecule that can act through Robo4 to inhibit Rac activation by VEGF. In still a further embodiment, a composition as described herein includes a ligand of a Robo4 receptor, wherein the ligand can act through Robo4 to initiate paxillin activation of GIT1. In another embodiment, a composition as described herein includes a ligand of a Robo4 receptor, wherein the ligand can act through Robo4 to activate Git1 inhibition of ARF6. In a further embodiment, a composition as described herein includes a ligand of a Robo4 receptor, wherein the ligand can act through Robo4 in a manner that results in one or more of the following preservation of endothelial barrier function, blocking of VEGF signaling downstream of the VEGF receptor, inhibition of vascular leak, inhibition of pathologic angiogenesis, signal inhibition of multiple angiogenic, permeability and inflammatory factors.

Where the composition of the present invention includes a ligand of Robo4, the ligand be any composition or molecule that binds the extracellular domain of Robo4. Alternatively, a ligand of Robo4 can be any composition or molecule that acts through the Robo4 receptor to inhibit Rac activation by VEGF. Even further, a ligand of Robo4 can be any composition or molecule that acts through the Robo4 receptor to activate Git1 inhibition of ARF6. Still further, a ligand of Robo4 can be any composition or molecule that acts through the Robo4 receptor to activate Paxillin activation of Git1. In another aspect, a ligand of Robo4 can be any composition or molecule that mimics the Robo4 receptor to activate Paxillin activation of Git1. In one embodiment, a ligand of Robo4 included in a composition according to the present description comprises an isolated polypeptide of about 5, 10, 15, 20, 25, 30, 40, 50, 60, 70, 80, 90, 100, 200, 300, 400 amino acids in length.

Where a composition as described herein includes a ligand of Robo4, such ligand can be a Slit ligand, such as Slit2 ligand, or a fragment or variant thereof that binds and activates Robo4. In specific embodiments, the Slit ligand, or fragment or variant thereof, binds to and activates Robo4 in a manner that results in one or more of the following: inhibition of Rac, inhibition of ARF6; preservation of endothelial barrier function; blocking of VEGF signaling downstream of the VEGF receptor; inhibition of vascular leak; inhibition of pathologic angiogenesis; and signal inhibition of multiple angiogenic, permeability and inflammatory factors. For example, the ligand of Robo4 can comprise an amino acid sequence selected from Slit1 (SEQ ID NO: 1), Slit2 (SEQ ID NO: 2), Slit3 (SEQ ID NO: 3), fragments of Slit1, such as the fragment represented by SEQ ID NO: 4, fragments of Slit2, such as the fragment represented by SEQ ID NO: 5, and fragments of Slit3, such as the fragment represented by SEQ ID NO: 6. In other embodiments, the ligand of Robo4 may be selected from the Slit2 ligands represented by SEQ ID NO: 7 through SEQ ID NO: 15. In particular, a Robo4 ligand according to the present description may be selected from Slit2N (SEQ ID NO: 7), the Slit2 represented by SEQ ID NO: 8, Slit2AP (SEQ ID NO: 9), Slit2 D1 (SEQ ID NO: 10), Slit2 D1-D2 (SEQ ID NO: 11), Slit2 D1-D3 (SEQ ID NO: 12), Slit2 D1-D4 (SEQ ID NO: 13), Slit2 D1-E5 (SEQ ID NO: 14), and Slit2 D1-E6 (SEQ ID NO: 15), or fragments thereof that bind Robo4. For example, in some embodiments, a fragment of such amino acid sequences can be at least about 5, 10, 15, 20, 25, 30, 40, 50, 60, 70, 80, 90, or 100 amino acids long. The ligand of Robo4 can comprise an amino acid sequence having at least about 70%, at least about 75%, at least about 80%, at least about 85%, at least about 90%, at least about 95%, or at least about 100% sequence identity to and amino acid sequence selected from an amino acid sequence selected from SEQ ID NO: 1, SEQ ID NO: 2, SEQ ID NO: 3, and any of SEQ ID NO: 4 through SEQ ID NO: 15, or a fragment thereof that interacts with Robo4 in a manner that results in one or more of the following: inhibition of Rac; inhibition of ARF6; preservation of endothelial barrier function; blocking of VEGF signaling downstream of the VEGF receptor; inhibition of vascular leak; inhibition of pathologic angiogenesis; and signal inhibition of multiple angiogenic, permeability and inflammatory factors.

In some embodiments, a Slit fragment suitable as a Robo4 ligand as described herein may comprise the N-terminal region of a Slit. For example, the ligand of Robo4 can comprise amino acids 1-1132 of Slit1 (SEQ ID NO: 4), amino acids 1-1119 of Slit2 (SEQ ID NO: 5), amino acids 1-1118 of Slit3 (SEQ ID NO: 6), or any of the n-terminal fragments illustrated in FIG. 23 and detailed SEQ ID NO: 7 through SEQ ID NO: 15. In particular embodiments, the ligand of Robo4 can comprise a polypeptide consisting essentially of an amino acid sequence selected from any one of SEQ ID NO: 4 through SEQ ID NO: 15. In some embodiments, as reflected in the amino acid sequences of SEQ ID NO: 7 through SEQ ID NO: 15, a Slit fragment included in a composition of the present invention does not comprise the N-terminal most amino acids. For example, in some embodiments, the amino acid sequence may lack about 5, 10, 15, 20, 25, 30, 40, 50, 60, 70, 80, 90, or 100 N-terminal amino acids of a natural Slit. In other embodiments, the Slit fragment may not comprise the C-terminal most about 5, 10, 15, 20, 25, 30, 40, 50, 60, 70, 80, 90, or 100 amino acids of a natural Slit.

For example, in particular embodiments, the ligand of Robo4 can comprise a polypeptide consisting essentially of amino acids 281-511 (SEQ ID NO: 16) of Slit1 or amino acids 271-504 of Slit2 (SEQ ID NO: 17). Thus, the ligand of Robo4 can comprise SEQ ID NO:15 or SEQ ID NO: 16 or a fragment thereof that binds Robo4. The ligand of Robo4 can comprise an amino acid sequence having at least about 70%, at least about 75%, at least about 80%, at least about 85%, at least about 90%, at least about 95%, or at least about 100% sequence identity to SEQ ID NO: 16 or SEQ ID NO: 17 or a fragment thereof that interacts with Robo4 in a manner that results in one or more of the following: inhibition of Rac; inhibition of ARF6; preservation of endothelial barrier function; blocking of VEGF signaling downstream of the VEGF receptor; inhibition of vascular leak; inhibition of pathologic angiogenesis; and signal inhibition of multiple angiogenic, permeability and inflammatory factors.

In one embodiment, a composition for modulating the activity of one or more ARFs according to the present description includes a small molecule active agent capable of modulating the activity of an upstream activator of one or more ARFs. In one such embodiment, the small molecule active agent promotes the availability, activation or activity of one or more ARF-GAPs as described herein. In another such embodiment, the small molecule active agent inhibits the availability, activation or activity of an ARF-GEF as described herein. In another such embodiment, the small molecule active agent inhibits the activity of a cytohesin in a manner that results in one or more of the following: inhibition of Rac; inhibition of ARF6; preservation of endothelial barrier function; blocking of VEGF signaling downstream of the VEGF receptor; inhibition of vascular leak; inhibition of pathologic angiogenesis; and signal inhibition of multiple angiogenic, permeability and inflammatory factors.

In yet another such embodiment, small molecule active agent inhibits the activity of a cytohesin selected from the ARNO family of cytohesins in a manner that results in one or more of the following: inhibition of Rac; inhibition of ARF6; preservation of endothelial barrier function; blocking of VEGF signaling downstream of the VEGF receptor; inhibition of vascular leak; inhibition of pathologic angiogenesis; and signal inhibition of multiple angiogenic, permeability and inflammatory factors. In another embodiment, the small molecule active agent inhibits the activity of ARNO in a manner that results in one or more of the following: inhibition of Rac; inhibition of ARF6; preservation of endothelial barrier function; blocking of VEGF signaling downstream of the VEGF receptor; inhibition of vascular leak; inhibition of pathologic angiogenesis; and signal inhibition of multiple angiogenic, permeability and inflammatory factors. It is to be understood that in each embodiment including a small molecule active agent, one or more active agents as described herein may be included in the composition.

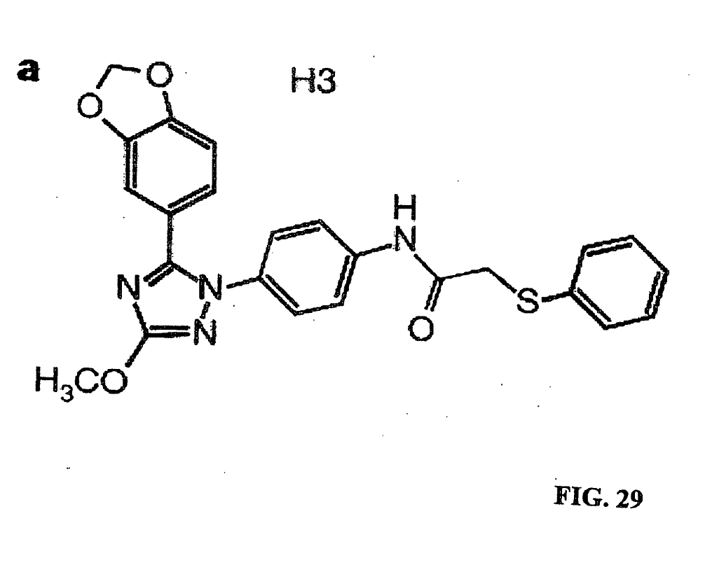

In a specific embodiment, a composition for inhibiting vascular permeability and/or pathologic angiogenesis includes SecinH3, the structure of which is provided in FIG. 29. SecinH3 is a known inhibitor of cytohesins (see, for example, Hafner et al., Inhibition of cytohesins by SecinH3 leads to hepatic insulin resistance, Nature (2006), 444, 941-944, and International Patent App. Publication No. WO 2006/053903). As is described in detail herein, in the present context, Secin-H3 inhibits ARF6 activation, VEGF induced ARF6-GTP, VEGF induced migration of endothelial cells, neovascular tuft formation in models of oxygen-induced retinopathy and choroidal neovascularization, and retinal hyperpermeability caused by VEGF. Thus, in one embodiment, a composition as described herein includes the SecinH3, which inhibits cytohesin activity, such as, for example the activity of ARNO, in a manner that results in one or more of the following: inhibition of Rac; inhibition of ARF6; preservation of endothelial barrier function; blocking of VEGF signaling downstream of the VEGF receptor; inhibition of vascular leak; inhibition of pathologic angiogenesis; and signal inhibition of multiple angiogenic, permeability and inflammatory factors.

In another embodiment, a composition as described herein includes one or more small molecule active agents selected from compounds that inhibit the availability, activation or activity of an ARF-GEF, such as a cytohesin, a cytohesin selected from the ARNO family of cytohesins, or ARNO in a manner that results in one or more of the following: inhibition of Rac; inhibition of ARF6; preservation of endothelial barrier function; blocking of VEGF signaling downstream of the VEGF receptor; inhibition of vascular leak; inhibition of pathologic angiogenesis; and signal inhibition of multiple angiogenic, permeability and inflammatory factors. In such an embodiment, a composition as described herein may include one or more compounds having the following chemical formula (Formula 1):

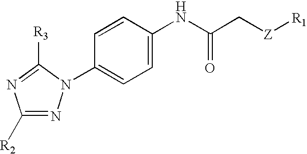

wherein:

R¹ and R³ are independently chosen from optionally substituted aryl, optionally substituted heteroaryl, optionally substituted cycloalkyl, or optionally substituted heterocycle;

R² is chosen from hydrogen, lower alkoxy, lower alkyl, halogen or hydroxy;

Z is chosen from O, S, NH, alkylene or a single bond; or

pharmaceutically acceptable salts, solvates or hydrates thereof. In one such embodiment, the one or more compounds are selected from compounds as described by Formula 1, wherein R³ is substituted with 1 to 5 substituents independently chosen from halogen, lower alkyl, lower alkoxy, heteroatom lower alkyl, hydroxy, or methylene dioxy, wherein two substituents together may form a fused cycloalkyl or heterocyclic ring structure. In another such embodiment, the one or more compounds are selected from compounds as described by Formula 1, wherein R¹ is chosen from unsubstituted aryl or unsubstituted heteroaryl; R² is chosen from hydrogen, lower alkoxy, or lower alkyl; R³ is chosen from aryl, optionally substituted with 1 to 5 substituents independently chosen from halogen, lower alkyl, lower alkoxy, or methylene dioxy, wherein two substituents together may form a fused cycloalkyl or heterocyclic ring structure; and Z is chosen from O, S, or a single bond.

In another embodiment, a composition as described herein includes one or more small molecule active agents selected from compounds that inhibit the availability, activation or activity of an ARF-GEF, such as a cytohesin, a cytohesin selected from the ARNO family of cytohesins, or ARNO in a manner that results in one or more of the following: inhibition of Rac; inhibition of ARF6; preservation of endothelial barrier function; blocking of VEGF signaling downstream of the VEGF receptor; inhibition of vascular leak; inhibition of pathologic angiogenesis; and signal inhibition of multiple angiogenic, permeability and inflammatory factors. In such an embodiment, a composition as described herein may include one or more compounds having the following chemical formula (Formula 2):

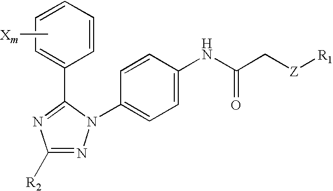

wherein:

R¹ is chosen from optionally substituted aryl, optionally substituted heteroaryl, optionally substituted cycloalkyl, or optionally substituted heterocycle;

R² is chosen from hydrogen, lower alkoxy, lower alkyl, halogen or hydroxy;

Z is chosen from O, S, NH, alkylene or a single bond;

X is independently chosen from halogen, lower alkyl, lower alkoxy, heteroatom lower alkyl, hydroxy, or methylene dioxy, wherein two substituents together may form a fused cycloalkyl or heterocyclic ring structure;

m is 0 to 5; or

pharmaceutically acceptable salts, solvates or hydrates thereof.

In one such embodiment, the one or more compounds are selected from the following compounds:

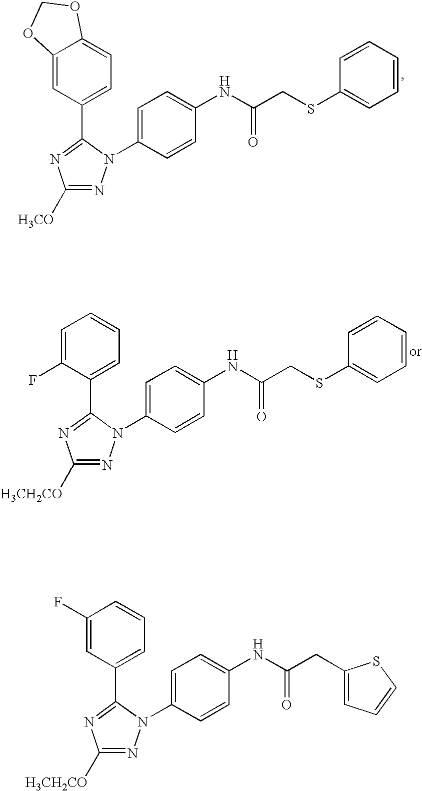

or pharmaceutically acceptable salts, solvates or hydrates thereof.

In another embodiment, a composition according to the present description includes a nucleic acid that directly or indirectly modulates the activity of a targeted molecule as described herein. Nucleic acids that may be included in composition as described herein may be selected from, for example, aptamers, antisense molecules, siRNA, ribozymes, and triple helix molecules. Techniques for the production and use of such molecules are known to those of skill in the art, such as described herein or in U.S. Pat. No. 5,800,998, incorporated herein by reference.

Antisense RNA and DNA molecules act to directly block the translation of mRNA by binding to targeted mRNA and preventing protein translation. With respect to antisense DNA, oligodeoxyribonucleotides derived from the translation initiation site, e.g., between the −10 and +10 regions of the target sequence are preferred. For example, an antisense RNA or DNA molecule may be included in a composition as described herein in a manner that reduces translation of one or more ARF proteins, including ARF6 or ARF1, or an upstream activator of an ARF protein, such as an ARF-GEF, including, for example, a cytohesin or a member of the ARNO family of cytohesins.

In one embodiment, a nucleic acid included in a composition as described herein is a small interfering RNA (siRNA) compounds or a modified equivalent thereof. In another embodiment, a nucleic acid included in a composition as described herein is a double-stranded small interfering RNA (siRNA) compound or a modified equivalent thereof. For example, an siRNA included in a composition as described herein may reduce levels of one or more ARF proteins, including ARF6 or ARF1, or an upstream activator of an ARF protein, such as an ARF-GEF, including, for example, a cytohesin or a member of the ARNO family of cytohesins.

As is generally known in the art, siRNA compounds are RNA duplexes comprising two complementary single-stranded RNAs of 21 nucleotides that form 19 base pairs and possess 3′ overhangs of two nucleotides (See, Elbashir et al., Nature 411:494 498 (2001); and PCT Publication Nos. WO 00/44895; WO 01/36646; WO 99/32619; WO 00/01846; WO 01/29058; WO 99/07409; and WO 00/44914). When appropriately targeted via its nucleotide sequence to a specific mRNA in cells, a siRNA can specifically suppress gene expression through a process known as RNA interference (RNAi) (See, e.g., Zamore & Aronin, Nature Medicine, 9:266 267 (2003)). siRNAs can reduce the cellular level of specific mRNAs, and decrease the level of proteins coded by such mRNAs. siRNAs utilize sequence complementarity to target an mRNA for destruction, and are sequence-specific. Thus, they can be highly target-specific, and in mammals have been shown to target mRNAs encoded by different alleles of the same gene. Because of this precision, side effects typically associated with traditional drugs may be reduced or eliminated. In addition, they are relatively stable, and like antisense and ribozyme molecules, they can also be modified to achieve improved pharmaceutical characteristics, such as increased stability, deliverability, and ease of manufacture. Moreover, because siRNA molecules take advantage of a natural cellular pathway, i.e., RNA interference, they are highly efficient in destroying targeted mRNA molecules

In-vivo inhibition of specific gene expression by RNAi has been achieved in various organisms including mammals. For example, Song et al., Nature Medicine, 9:347 351 (2003) demonstrate that intravenous injection of Fas siRNA compounds into laboratory mice with autoimmune hepatitis specifically reduced Fas mRNA levels and expression of Fas protein in mouse liver cells. The gene silencing effect persisted without diminution for 10 days after the intravenous injection. The injected siRNA was effective in protecting the mice from liver failure and fibrosis. Song et al., Nature Medicine, 9:347 351 (2003). Several other approaches for delivery of siRNA into animals have also proved to be successful (See, e.g., McCaffery et al., Nature, 418:38 39 (2002); Lewis et al., Nature Genetics, 32:107 108 (2002); and Xia et al., Nature Biotech., 20:1006 1010 (2002)).

The siRNA compounds provided according to the present description can be synthesized using conventional RNA synthesis methods. For example, they can be chemically synthesized using appropriately protected ribonucleoside phosphoramidites and a conventional DNA/RNA synthesizer. Various applicable methods for RNA synthesis are disclosed in, e.g., Usman et al., J. Am. Chem. Soc., 109:7845 7854 (1987) and Scaringe et al., Nucleic Acids Res., 18:5433 5441 (1990). Custom siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK).

A composition as described herein may be prepared as a pharmaceutical formulation. For example, in addition to one or more small molecules, proteins, peptides or nucleic acids, a composition as described may include a pharmaceutically acceptable carrier to provide a formulation that is suitable for therapeutic administration. As used herein, “pharmaceutically acceptable” refers to a material that is not biologically or otherwise undesirable, i.e., the material may be administered to a subject, along with the desired composition (e.g., a desired ligand, protein, peptide, nucleic acid, small molecule therapeutic, etc.), without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

A pharmaceutical composition according to the present description may be prepared in any for suitable for administration, such as a tableted composition, a powder composition for encapsulation, a solution composition for encapsulation or parenteral delivery, an emulsion, or a suspension, such as a formulation that incorporates is incorporated into microparticles, a matrix material or liposomes. A pharmaceutical composition as described herein may include components that targeted to a particular cell type via antibodies, receptors, or receptor ligands. The following references are examples formulation technologies targeting specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Pharmaceutical carriers and their formulations are described, for example, in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered. In addition to one or more carriers, a pharmaceutical composition as described herein may include one or more thickener, flavoring, diluent, buffer, preservative, antimicrobial agents, antiinflammatory agents, anesthetics, surface active agent, and the like.

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, transdermally orally, parenterally (e.g., intravenously), intratracheally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained. See, e.g., U.S. Pat. No. 3,610,795, which is incorporated by reference herein.

The compositions disclosed herein may be administered prophylactically to patients or subjects who are at risk for vascular permeability or pathologic angiogenesis. Thus, the method can further comprise identifying a subject at risk for vascular permeability or pathologic angiogenesis prior to administration of the herein disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389

### Methods

Methods of screening for or evaluating an agent that inhibits cellular protrusive activity, vascular leak, endothelial permeability, and/or pathologic angiogenesis are provided herein. In one embodiment, the method comprises determining the ability of said agent to affect the activation or activity of GIT1, including Robo4-mediated activation of GIT1. For example, Robo4-mediated activation of Git1 can be determined by the steps comprising: contacting a first cell expressing Robo4 with a candidate agent, contacting a second cell essentially identical to the first cell but substantially lacking Robo4 with the candidate agent, and assaying for GIT1 activation in the first and second cells, wherein detectably higher Git1 activation in the first cell as compared to the second cell indicates Robo4-mediated Git1 activation by said agent.

As disclosed herein, Robo4-mediated Git1 activation results in ARF6 inactivation. ARF6 is involved in VEGF-mediated activation of Rac, which activates Pak, which activates MEK, which activates ERK, which promotes vascular permeability. Thus, as disclosed herein GIT1 activation can be assayed by detecting any of the components of the signaling pathway that is either activated or inactivated, and Robo4-mediated GIT1 activation can be assayed by detecting ARF6 inactivation, Rac inactivation, Pak inactivation, MEK inactivation, or ERIC inactivation. It is understood that any other known or newly discovered method of monitoring this signaling pathway can be used in the disclosed methods.

Also provided is a method of screening for or evaluating an agent that inhibits cellular protrusive activity, vascular leak, endothelial permeability, and/or pathologic angiogenesis, comprising determining the ability of said agent to inhibit ARF6, Rac, Pak, MEK, or ERK. For example, in one embodiment, Robo4-mediated inhibition of ARF6, Rac, Pak, MEK, or Erk is determined by the steps comprising: contacting a first cell expressing Robo4 with a candidate agent, contacting a second cell essentially identical to the first cell but substantially lacking Robo4 with the candidate agent, assaying for inhibition of ARF6, Rac, Pak, MEK, ERK, or a combination thereof, in the first and second cells, wherein detectably lower ARF6, Rac, Pak, MEK, or ERK activation in the first cell as compared to the second cell indicates Robo4-mediated ARF6, Rac, Pak, MEK, or ERIC inhibition by said agent. Alternatively, the ability of an agent to inhibit ARF6, Rac, Pak, MEK, or ERK in the absence of Robo4 signaling may also be determined. In one such embodiment, the method comprises: contacting a first cell is with a candidate agent; contacting a second cell identical to the first cell with a control lacking the candidate agent; and assaying for inhibition ARF6, Rac, Pak, MEK, ERK, or a combination thereof, in the first and second cells, wherein detectably lower ARF6, Rac, Pak, MEK, or ERK activation in the first cell as compared to the second cell indicates inhibition of ARF6, Rac, Pak, MEK, or ERK inhibition by said agent.

Activation of signaling proteins such as Rac, Pak, MEK, ERK can be assayed by detecting the phosphorylation of said proteins. Cell-based and cell-free assays for detecting phosphorylation of proteins are well known in the art and include the use of antibodies, including, for example, anti-Phosphoserine (Chemicon® AB1603) (Chemicon, Temecula, Calif.), anti-Phosphothreonine (Chemicon® AB1607), and anti-Phosphotyrosine (Chemicon® AB1599). Site-specific antibodies can also be generated specific for the phosphorylated form of DDX-3. The methods of generating and using said antibodies are well known in the art.

The herein disclosed assay methods can be performed in the substantial absence of VEGF, TNF, thrombin, or histamine. Alternatively, the disclosed assay methods can be performed in the presence of a biologically active amount of VEGF, TNF, thrombin, or histamine.

“Activities” of a molecule, such as a protein or peptide molecule, include, for example, transcription, translation, intracellular translocation, secretion, phosphorylation by kinases, cleavage by proteases, homophilic and heterophilic binding to other proteins, ubiquitination.

In one embodiment, the method of screening described herein is a screening assay, such as a high-throughput screening assay. Thus, the contacting step can be in a cell-based or cell-free assay. For example, vascular endothelial cells can be contacted with a candidate agent either in vivo, ex vivo, or in vitro. The cells can be on in monolayer culture but preferably constitute an epithelium. The cells can be assayed in vitro or in situ or the protein extract of said cells can be assayed in vitro for the detection of activated (e.g., phosphorylated) Rac, Pak, MEK, ERK. Endothelial cells can also be engineered to express a reporter construct, wherein the cells are contacted with a candidate agent and evaluated for reporter expression. Other such cell-based and cell-free assays are contemplated for use herein.

In a specific embodiment, a method for identifying an agent that inhibits cellular protrusive activity, vascular leak, endothelial permeability, and/or pathologic angiogenesis involves an aptamer-displacement screen assay as described, for example, by Hafner et al. (Displacement of protein-bound aptamers with small molecules screened by fluorescence polarization, Nat Protoc (2008), 3, 579-587). In particular, such a method can be used to identify and confirm the activity of small molecules, such as those described herein, for inhibiting the activity of a targeted ARF-GEF, such as a cytohesin, a cytohesin belonging to the ARNO family of cytohesins or ARNO. In confirming such activity, active agents capable of inhibiting cellular protrusive activity, vascular leak, endothelial permeability, and/or pathologic angiogenesis can be identified. Such an aptamer-displacement screen assay utilizes displacement of a fluorescence-labeled aptamer protein inhibitor to identify small molecules with activity analogous to the fluorescence-labeled aptamer protein inhibitor. The association of the aptamer with its target is detected by fluorescence polarization. The fluorescence-labeled aptamer exhibits low polarization in the non-bound state. When bound to the target protein, the fluorescence polarization of the fluorescence-labeled aptamer is increased. If a small molecule displaces the aptamer from the protein, the fluorescence polarization of the fluorescence-labeled aptamer decreases, thereby allowing identification of small molecule candidates exhibiting activities analogous to the fluorescence labeled aptamer.

In general, candidate agents can be identified from libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including. Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits vascular permeability. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate vascular permeability.

Methods for inhibiting cellular protrusive activity, vascular leak, endothelial permeability, and/or pathologic angiogenesis in a subject are also provided herein. As is detailed herein, activation of Robo4 inhibits or reduces the activation of ARF6, and thereby inhibits vascular permeability. As described herein, activation of Robo4 signaling achieves such effects through initiation of paxillin activation of GIT1, which, in turn, leads to GIT1 inhibition of ARF6. Therefore, in one embodiment, a method for inhibiting cellular protrusive activity, vascular leak, endothelial permeability, and/or pathologic angiogenesis as described herein comprises administering a therapeutically effective amount of a composition as described herein to a subject in need thereof. In one such embodiment, the method includes administering a therapeutically effective amount of a Robo4 ligand according to the present description to achieve an effect selected from one or more of inhibition of Rac, inhibition of Rac activation by VEGF, preservation of endothelial cell barrier function, inhibition of VEGF signaling downstream of the VEGF receptor, inhibition of vascular leak, and inhibition of multiple angiogenic, permeability and inflammatory factors.

However, as is further described herein, the inhibition of vascular permeability or pathologic angiogenesis resulting from Robo4 signaling can also be achieved without activation of Robo4, in particular, modulation of one or more downstream steps in the Robo4 signaling pathway described herein can also inhibit cellular protrusive activity, vascular leak, endothelial permeability, and/or pathologic angiogenesis. In one such embodiment, a method as described herein comprises modulating one or more of the steps in the Robo4 signaling pathway such to achieve an effect selected from one or more of inhibition of Rac, inhibition of Rac activation by VEGF, preservation of endothelial cell barrier function, inhibition of VEGF signaling downstream of the VEGF receptor, inhibition of vascular leak, and inhibition of multiple angiogenic, permeability and inflammatory factors.

In one such embodiment, a method as described herein comprises directly or indirectly inhibiting activation of ARF6. For example, in one embodiment, a method for inhibiting cellular protrusive activity, vascular leak, endothelial permeability, and/or pathologic angiogenesis includes inhibiting an upstream activator of ARF6. In one such embodiment, the method includes inhibiting the activity of one or more ARF-GEF or other cytohesin family GEFs such that the activity of one or more protein of the ARF family of proteins is reduced. For example, the method may include providing a composition including one or more molecules that decrease the activity, activation or availability of a cytohesin, such as ARNO or a cytohesin belonging to the ARNO family of cytohesins, such that fewer ARF6 proteins are in a GTP-bound state, thereby reducing the pool of active ARF proteins. In another such embodiment, the method includes promoting the activity of an upstream inhibitor of ARF6. In one such embodiment, the method includes increasing the activity of one or more ARF-GAP such that the activity or activation of one or more protein from the ARF family of proteins is reduced. For example, the method may include providing a composition that includes one or more molecules that increase the activity or availability of individual Git1 proteins such that fewer ARF proteins are activated, thereby reducing a signal cascade acting through or propogated by the ARF proteins. In such embodiments, where activity of the ARF family of proteins is targeted, the ARF protein(s) affected may be selected from, for example, ARF6 and ARF1.

In each of the methods for inhibiting cellular protrusive activity, vascular leak, endothelial permeability, and/or pathologic angiogenesis, a composition as described herein may be used to directly inhibit ARF6 activity, to inhibit an upstream activator of ARF6, or to promote an upstream inhibitor of ARF6. In a specific embodiment, a method for inhibiting cellular protrusive activity, vascular leak, endothelial permeability, and/or pathologic angiogenesis comprises inhibiting ARF6 activity by administration of a small molecule, protein, peptide or nucleic acid as described herein. In another embodiment, a method for inhibiting vascular permeability or pathologic angiogenesis comprises inhibiting ARF6 activity by administration of an activator of an ARF-GAP, such as Git1. In yet another embodiment, a method for inhibiting cellular protrusive activity, vascular leak, endothelial permeability, and/or pathologic angiogenesis comprises administering an inhibitor of ARF6 activation of Rac.

In specific embodiments, a method for vascular leak or endothelial permeability as described herein includes inhibiting cellular protrusive activity, vascular leak, endothelial permeability, and/or pathologic angiogenesis experienced by a subject that is associated with a disease state selected from infectious and non-infectious diseases that may result in a cytokine storm, graft versus host disease (GVHD), adult respiratory distress syndrome (ARDS), sepsis, avian influenza, smallpox, and systemic inflammatory response syndrome (SIRS), ischemia/reperfusion injury following stroke or myocardial infarction, edema associated with brain tumors, ascites associated with malignancies, Meigs' syndrome, lung inflammation, nephrotic syndrome, pericardial effusion and pleural effusion, inflammation, allergic diseases, cancer, cerebral stroke, myocardial infarction, pulmonary and cardiac insufficiency, renal failure, and retinopathies.

Additionally, in specific embodiment, a method for inhibiting pathologic angiogenesis as described herein includes inhibiting pathologic angiogenesis experienced by a subject that is associated with a disease state selected from hemangioma, solid tumors, leukemia, metastasis, telangiectasia psoriasis scleroderma, pyogenic granuloma, myocardial angiogenesis, plaque neovascularization, coronary collaterals, ischemic limb angiogenesis, corneal diseases, rubeosis, neovascular glaucoma, diabetic retinopathy (DR), retrolental fibroplasia, non-proliferative diabetic macular edema (DME), arthritis, diabetic neovascularization, age-related macular degeneration (AMD), retinopathy of prematurity (ROP), ischemic retinal vein occlusion (IRVO), wound healing, peptic ulcer, fractures, keloids, vasculogenesis, hematopoiesis, ovulation, menstruation, and placentation.

In another embodiment, a method of treating or preventing avian flu is provided, wherein the method comprises identifying a subject having or at risk of having said avian flu, and administering to the subject a therapeutically effective amount of a composition as described herein.

In another embodiment, a method of treating or preventing adult respiratory distress syndrome (ARDS) is provided, wherein the method comprises identifying a subject having or at risk of having said ARDS, and administering to the subject a therapeutically effective amount of a composition as described herein.

In another embodiment, a method of treating or preventing systemic inflammatory response syndrome (SIRS) is provided, wherein the method comprises identifying a subject having or at risk of having said SIRS, and administering to the subject a therapeutically effective amount of a composition as described herein.

In another embodiment, a method of treating or preventing graft versus host disease (GVHD) is provided, wherein the method comprises identifying a subject having or at risk of having said RDS, and administering to the subject a therapeutically effective amount of a composition as described herein.

In another embodiment, a method of treating or preventing tumor formation or growth is provided, wherein the method comprises identifying a subject having or at risk of having said tumor formation or growth, and administering to the subject a therapeutically effective amount of a composition as described herein.

In another embodiment, a method of treating or preventing respiratory distress syndrome (RDS) is provided, wherein the method comprises identifying a subject having or at risk of having said RDS, and administering to the subject a therapeutically effective amount of a composition as described herein.

In another embodiment, a method of treating or prevention ischemic retinal vein occlusion (IRVO) in a subject is provided, wherein the method comprises identifying a subject having or at risk of having said IRVO, and administering to the subject a therapeutically effective amount of a composition as described herein.

In another embodiment, a method of treating or preventing non-proliferative diabetic macular edema (DME) in a subject is provided, wherein the method comprises identifying a subject having or at risk of having said DME, and administering to the subject a therapeutically effective amount of a composition as described herein.

In another embodiment, a method of treating or preventing retinopathy of pre-maturity (ROP) is provided, wherein the method comprises identifying a subject having or at risk of having said ROP, and administering to the subject a therapeutically effective amount of a composition as described herein.

In another embodiment, a method of treating or preventing diabetic retinopathy (DR) in a subject is provided, wherein the method comprises identifying a subject having or at risk of having said DR, and administering to the subject a therapeutically effective amount of a composition as described herein.

In another embodiment, a method of treating or preventing wet macular degeneration (AMD) in a subject is provided, wherein the method comprises identifying a subject having or at risk of having said AMD, and administering to the subject a therapeutically effective amount of a composition as described herein.

In another embodiment, a method of treating or preventing ischemia in a subject is provided, wherein the method comprises identifying a subject having or at risk of having said ischemia, and administering to the subject a therapeutically effective amount a composition as described herein.

In another embodiment, a method of treating or preventing hemorrhagic stroke in a subject is provided, wherein the methods comprises identifying a subject having or at risk of having said hemorrhagic stroke, and administering to the subject a therapeutically effective amount of a composition as described herein.

In another embodiment, a method of treating or preventing reperfusion injury, such as that observed in myocardial infarction and stroke, in a subject is provided, wherein the method comprises identifying a subject having or at risk of having said reperfusion injury, and administering to the subject a therapeutically effective amount of a composition as described herein.

In another embodiment, a method of treating or preventing a dermal vascular blemish or malformation in a subject is provided, wherein the method comprises identifying a subject having or at risk of having said blemish, and administering to the skin of the subject a therapeutically effective amount of a composition as described herein.

In some aspects, subjects are identified by medical diagnosis. For example, subjects with diabetic retinopathy and macular degeneration can be identified by visualization of excess blood vessels in the eyes. Acute lung injury can be diagnosed by lung edema in the absence of congestive heart failure. Ischemic stroke can be diagnosed by neurologic presentation and imaging (MRI and CT). Other known or newly discovered medical determinations can be used to identify subjects for use in the disclosed methods.

In addition, subjects can be identified by genetic predisposition. For example, genes that predispose patients to age related macular degeneration have been identified (Klein R J, et al, 2005; Yang Z, et al. 2006; Dewan A, et al. 2006). Likewise, genetic mutations that predispose patients to vascular malformations in the brain have been identified (Plummer N W, et al., 2005). Other known or newly discovered genetic determinations can be used to identify subjects for use in the disclosed methods.

### EXAMPLES

The Examples that follow are offered for illustrative purposes only and are not intended to limit the scope of the compositions and methods described herein in any way. In each instance, unless otherwise specified, standard materials and methods were used in carrying out the work described in the Examples provided. All patent and literature references cited in the present specification are hereby incorporated by reference in their entirety.

The practice of the present invention employs, unless otherwise indicated, conventional techniques of chemistry, molecular biology, microbiology, recombinant DNA, genetics, immunology, cell biology, cell culture and transgenic biology, which are within the skill of the art (See, e.g., Maniatis, T., et al. (1982) Molecular Cloning: A Laboratory Manual (Cold Spring Harbor Laboratory, Cold Spring Harbor, N.Y.); Sambrook, J., et al. (1989) Molecular Cloning: A Laboratory Manual, 2^{nd} Ed. (Cold Spring Harbor Laboratory, Cold Spring Harbor, N.Y.); Ausubel, F. M., et al. (1992) Current Protocols in Molecular Biology, (J. Wiley and Sons, NY); Glover, D. (1985) DNA Cloning, I and II (Oxford Press); Anand, R. (1992) Techniques for the Analysis of Complex Genomes, (Academic Press); Guthrie, G. and Fink, G. R. (1991) Guide to Yeast Genetics and Molecular Biology (Academic Press); Harlow and Lane (1988) Antibodies: A Laboratory Manual (Cold Spring Harbor Laboratory, Cold Spring Harbor, N.Y.); Jakoby, W. B. and Pastan, I. H. (eds.) (1979) Cell Culture. Methods in Enzymology, Vol. 58 (Academic Press, Inc., Harcourt Brace Jovanovich (NY); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Hogan et al. (eds) (1994) Manipulating the Mouse Embryo. A Laboratory Manual, 2^{nd} Edition, Cold Spring Harbor Laboratory Press, Cold Spring Harbor, N. Y. A general discussion of techniques and materials for human gene mapping, including mapping of human chromosome 1, is provided, e.g., in White and Lalouel (1988) Ann. Rev. Genet. 22:259 279. The practice of the present invention employs, unless otherwise indicated, conventional techniques of chemistry, molecular biology, microbiology, recombinant DNA, genetics, and immunology. (See, e.g., Maniatis et al., 1982; Sambrook et al., 1989; Ausubel et al., 1992; Glover, 1985; Anand, 1992; Guthrie and Fink, 1991).

Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

### Example 1

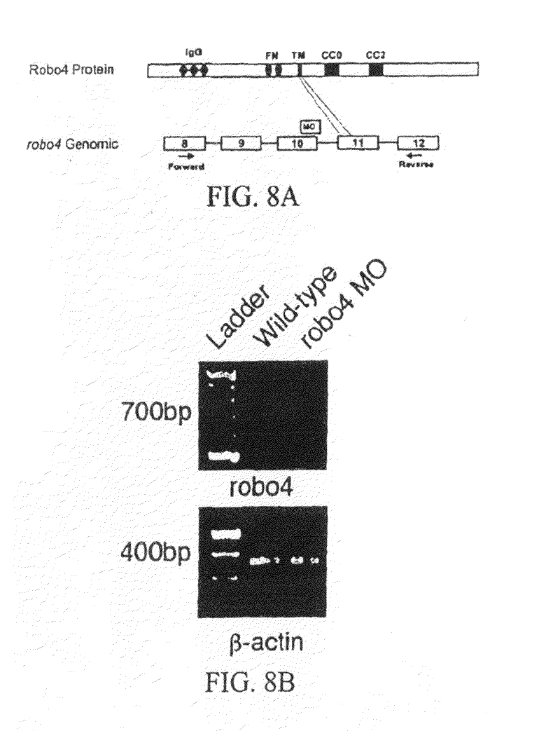

Robo4 is Required for Vascular Guidance in vivo: During the past decade, the zebrafish has become an attractive model for analysis of vascular development (Weinstein, 2002), and was chosen to investigate the biological importance of Robo4 in vivo. To suppress Robo4 gene expression, a previously described splice-blocking morpholino that targets the exon10-intron10 boundary of Robo4 pre-mRNA (Bedell et al., 2005) was used. To verify the efficacy of the Robo4 morpholino, RNA was isolated from un-injected and morpholino-injected embryos, and analyzed by RT-PCR with primers flanking the targeted exon (FIG. 8A). Injection of the Robo4 morpholino resulted in complete loss of wild-type RNA when compared to the un-injected control, indicating that morphant zebrafish are functionally null for Robo4 (FIG. 8B).

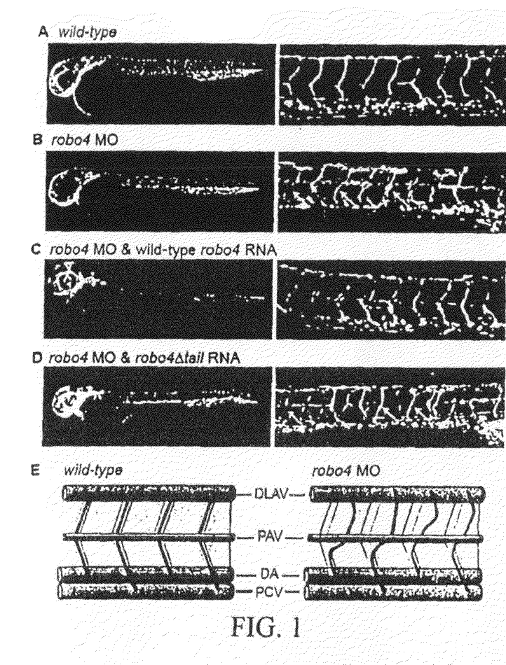

TG(fli1:egfp)^{yl} zebrafish embryos, which express green fluorescent protein under the control of the endothelial specific fli1 promoter, and permit detailed visualization of the developing endothelium in vivo were utilized to evaluate the consequence of morpholino-mediated knockdown of Robo4 on vascular development (FIG. 1A; Lawson and Weinstein, 2002). At 48 hpf, Robo4 MO-injected embryos exhibited wild-type formation of the primary axial vessels (dorsal aorta and posterior cardinal vein), as well as the dorsal longitudinal anastomotic vessel and parachordal vessel, indicating that vasculogenesis and angiogenesis, respectively, are not affected by reduction of Robo4 levels (FIG. 1B, right panel). However, a striking degree of abnormality was observed in the architecture of the intersegmental vessels in Robo4 morphants. In wild-type embryos, the intersegmental vessels arise form the dorsal aorta and grow toward the dorsal surface of the embryo, tightly apposed to the somitic boundary. It is this precise trajectory between the somites that defines the characteristic chevron shape of the intersegmental vessels (FIG. 1A, right panel). Rather than adopting this stereotypical pattern, the intersegmental vessels of Robo4 morphant embryos grew the wrong direction (FIG. 1B, right panel: white arrows indicate abnormal vessels). At 48 hpf, 60% of embryos injected with the Robo MO exhibited this defect, compared to 5% in wild-type embryos. Importantly, Robo4 morphants were indistinguishable from control embryos by phase microscopy, indicating that the observed vascular patterning defects were not a result of gross morphological perturbation. Together, these data demonstrate a requirement for Robo4 during zebrafish vascular development and suggest that functional output from the receptor elicits a repulsive guidance cue.

### Example 2

The Robo4 Cytoplasmic Tail is required for Vascular Guidance in vivo: It was next determined whether the vascular defects observed in Robo4 morphants could be suppressed by reconstitution of robo4. robo4 MO and wildtype murine Robo4 RNA, which is refractory to the morpholino, were injected into TG(fli1:egfp)yl embryos and vascular patterning was analyzed at 48. hpf. Robo4 RNA restored the stereotypic patterning of the trunk vessels in approximately 60% of morphant embryos, confirming the specificity of gene knockdown (FIGS. 1B and C, right panels).

The ability of the robo4 to regulate vascular development is likely a consequence of its ability to transmit cytoplasmic signals. To substantiate this notion, Robo4 MO and a mutant form of murine Robo4 lacking the portion of the receptor that interacts with cytoplasmic components (robo4Δtail) was co-injected and vessel architecture evaluated at 48 hpf. Unlike wild-type Robo4 RNA, robo4Δtail was unable to rescue patterning defects in morphant embryos (FIGS. 1B and D, right panels). These data demonstrate that information contained in the cytoplasmic tail of Robo4 is critical for vascular guidance during zebrafish embryogenesis. All together, these in vivo analyses indicate that Robo4 activity is required for precisely defining the trajectory of the intersegmental vessels during vertebrate vascular development (FIG. 1E).

### Example 3

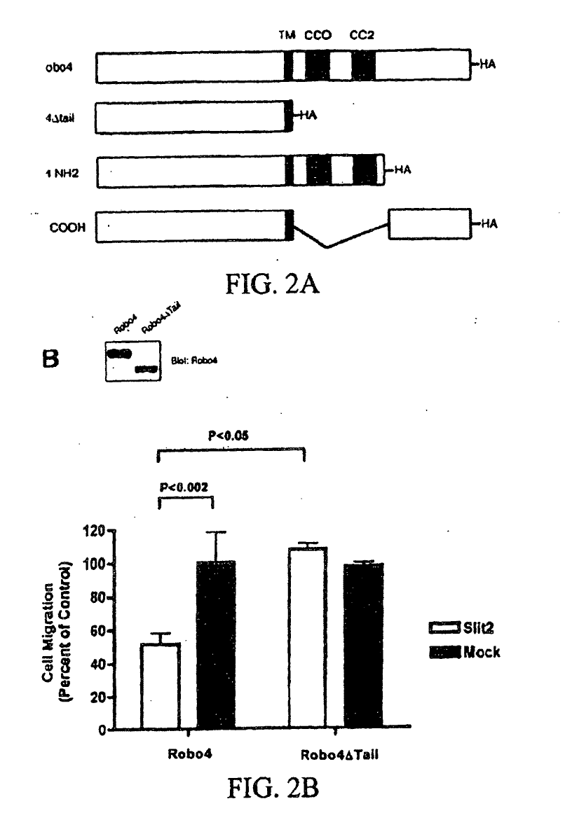

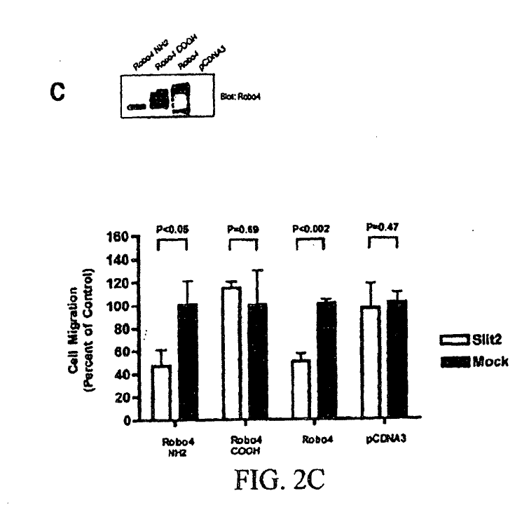

The Robo4 Cytoplasmic Tail is required for Inhibition of Haptotaxis: Slit2-Robo4 signaling inhibits migration of primary endothelial cells towards a gradient of VEGF, and of HEK 293 cells ectopically expressing Robo4 towards serum (Park et al., 2003; Seth et al., 2005). In addition to soluble growth factors, immobilized extracellular matrix proteins such as fibronectin play a critical role in cellular motility (Ridley et al., 2003), and gradients of fibronectin can direct migration in a process called haptotaxis. Indeed it has been shown that fibronectin is deposited adjacent to migrating endothelial cells in the early zebrafish embryo (Jin et al., 2005). The observation that Robo4 is required for proper endothelial cell migration in vivo (FIG. 1), indicated the ability of Slit2-Robo4 signaling to modulate fibronectin-induced haptotaxis. HEK 293 cells were transfected with Robo4 or Robo4ΔTail (FIG. 2A) and subjected to haptotaxis migration assays on membranes coated with a mixture of fibronectin and Slit2 (Slit2N (SEQ ID NO: 7)). Slit2 inhibited fibronectin-induced migration of cells expressing Robo4, but not Robo4ΔTail, demonstrating that the Robo4 cytoplasmic tail is critical for repulsive activity of the receptor (FIG. 2B).

The region of the Robo4 cytoplasmic tail that is required for inhibition of cell migration was next defined. HEK 293 cells were transfected with Robo4 deletion constructs (FIG. 2A) and subjected to haptotaxis migration assays. Fibronectin-dependent migration of cells expressing Robo4-NH2, but not Robo4-COOH was inhibited by Slit2 (FIG. 2C), demonstrating that the N-terminal half of the Robo4 cytoplasmic tail is necessary and sufficient for modulation of cell motility.

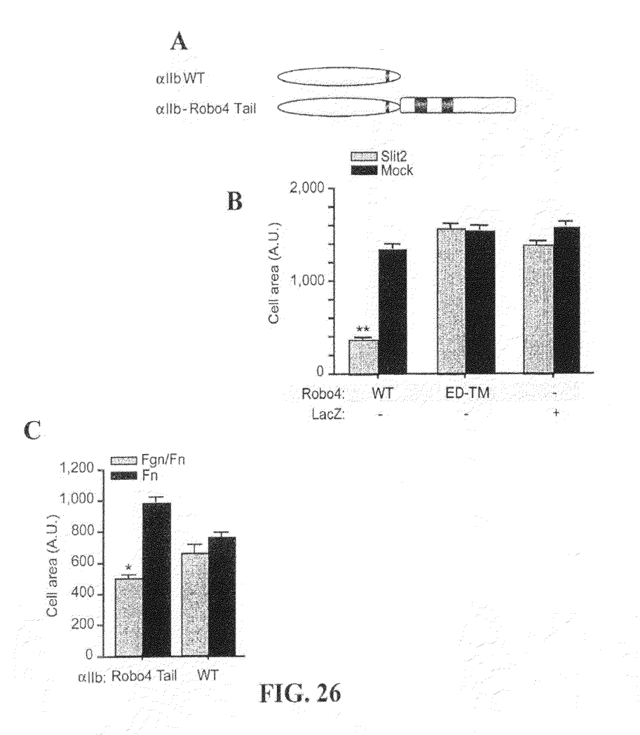

To assess whether Slit2-Robo4 signaling inhibited spreading, we transfected HEK 293 cells with Robo4, Robo4 ED-TM or empty vector (pcDNA3) and subjected them to adhesion and spreading assays on fibronectin. Although cells expressing Robo4 adhered normally to coverslips coated with fibronectin and Slit2 (data not shown), they spread significantly less than cells transfected with Robo4 ED-TM or pcDNA3 (FIG. 26B), indicating that Slit2-Robo4 signaling modulates intracellular pathways that control cell spreading.

The inability of Robo4 ED-TM to inhibit spreading showed that the Robo4 cytoplasmic domain is required for this activity; to test whether it is sufficient for inhibition of spreading, we generated an αIIb Integrin-Robo4 chimeric protein, in which the cytoplasmic domain of Robo4 was fused to the C-terminal tail of the integrin αIIb subunit (αIIb-Robo4), thus enabling us to initiate Robo4 signaling with fibrinogen, a ligand for αIIbβ3 integrin. We plated Chinese Hamster Ovary (CHO-K1) cells expressing αIIb and β3 subunits (αIIbβ3) or αIIb-Robo4 and β3 subunits (αIIb-Robo4:β3) on fibronectin alone or a mixture of fibronectin/fibrinogen. αIIb:β3 expressing cells spread to the same extent on either matrix, while spreading of αIIb-Robo4:β3 expressing cells was significantly inhibited in the presence of fibrinogen (FIG. 26C).

We next defined the region of the Robo4 cytoplasmic tail that is required for inhibition of cell migration. HEK 293 cells were transfected with Robo4 deletion constructs (FIG. 26A) and subjected to haptotaxis migration assays. Fibronectin-dependent migration of cells expressing Robo4-NH2, but not Robo4-COOH was inhibited by Slit2 (Slit2N (SEQ ID NO: 7)) (FIG. 2C), demonstrating that the N-terminal half of the Robo4 cytoplasmic tail is necessary and sufficient for modulation of cell motility.

### Example 4

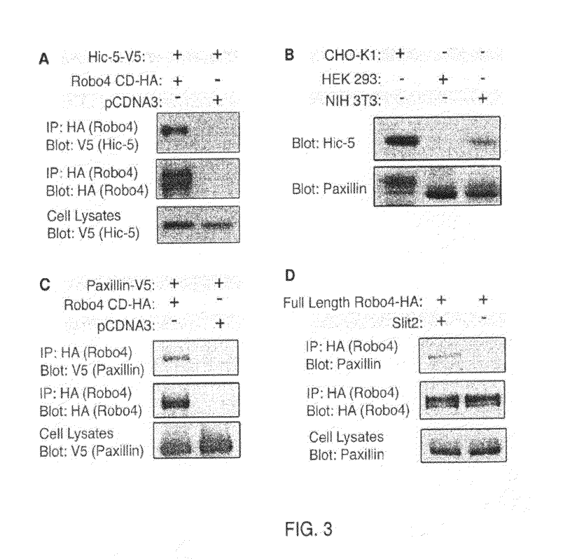

Paxillin Family Members are Robo4-interacting Proteins: Identification of the region of the Robo4 cytoplasmic tail that confers functional activity allowed the search for cytoplasmic components that might regulate Robo4 signal transduction. Using the N-terminal half of the Robo4 tail as a bait, a yeast two-hybrid screen of a human aortic cDNA library was performed, which identified a member of the paxillin family of adaptor proteins, Hic-5, as a potential Robo4-interacting protein (FIG. 8). To verify this interaction, Hic-5 plasmids were isolated and re-transformed into yeast with Robo4 or empty vector. Only strains co-expressing Robo4 and Hic-5 were competent to grow on nutrient deficient medium and induce robust betagalactosidase activity (FIG. 8B). To further confirm this interaction, co-immunoprecipitation experiments were performed using mammalian cells co-transfected with Hic-5 and the Robo4 cytoplasmic tail. Hic-5 was found in anti-Robo4 immunoprecipitates of HEK 293 cells expressing Robo4 and Hic-5, but not Hic-5 alone (FIG. 3A). Collectively, these data demonstrate that Hic-5 specifically interacts with the Robo4 cytoplasmic tail in both yeast and mammalian cells.

Hic-5 and its paralog, paxillin, can exhibit cell-type specific expression (Turner, 2000; Yuminamochi et al., 2003). For this reason, it was determined which of these proteins were expressed in HEK 293 cells, the cell line used in the haptotaxis migration assays. Western blotting of cell lysates from CHO-K1, HEK 293 and NIH3T3 cells with antibodies to Hic-5 or paxillin detected paxillin in all cell lines, whereas Hic-5 was only found in CHO-K1 and NIH3T3 cells (FIG. 3B). This not only suggested that Hic-5 and paxillin could interact with Robo4 to regulate cell migration, but that paxillin was the likely binding partner in HEK 293 cells. With this latter idea in mind, co-immunoprecipitation experiments were performed using mammalian cells expressing paxillin and the Robo4 cytoplasmic tail. As was observed with Hic-5, paxillin was identified in anti-Robo4 immunoprecipitates of HEK 293 cells expressing paxillin and Robo4, but not paxillin alone (FIG. 3C).

Since Slit2 is a physiological ligand of Robo4 (Park et al., 2003; Hohenester et al., 2006), it was determined whether Slit2 stimulation regulated the interaction between Robo4 and paxillin. HEK 293 cells expressing Robo4 were incubated in the presence or absence of Slit2 (Slit2N (SEQ ID NO: 7)). In the presence of Slit2, endogenous paxillin was detected in Robo4 immunoprecipitates. In sharp contrast, in the absence of Slit2, no paxillin was detected in the immunoprecipitates (FIG. 3E). Thus, engagement of Robo4 by Slit2 stimulated its association with paxillin.

### Example 5

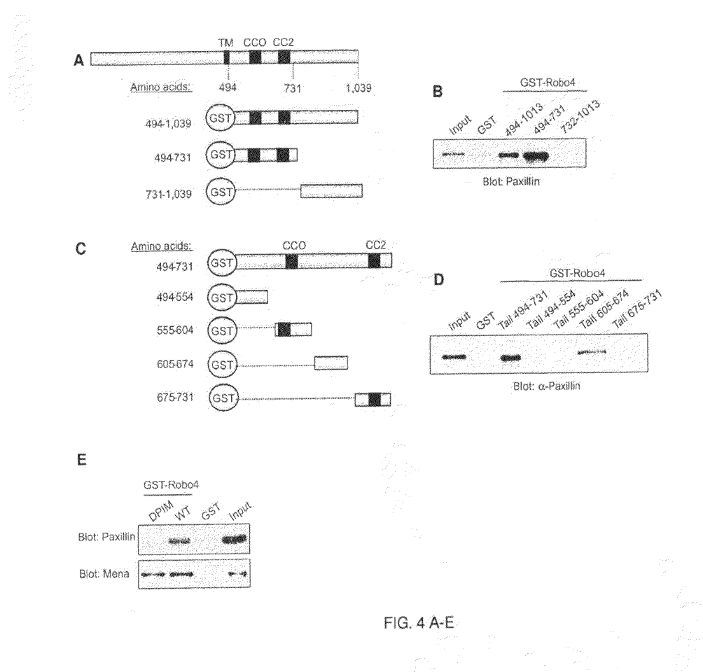

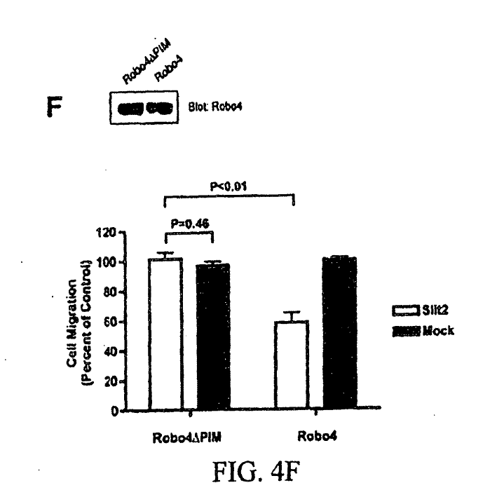

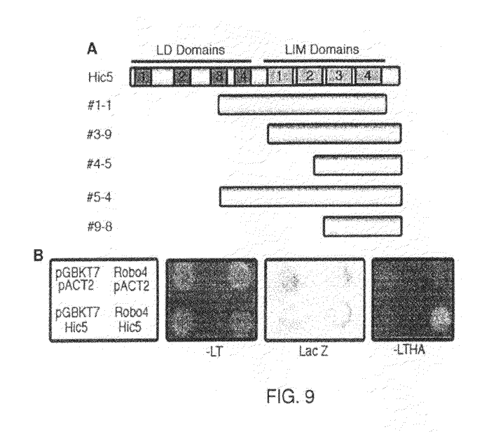

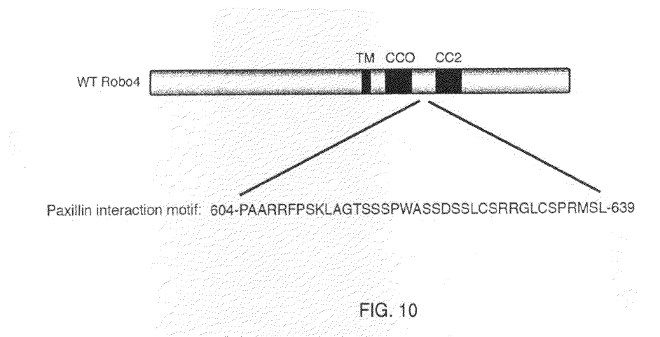

Identification of the Paxillin Interaction Motif of Robo4: To precisely define the region of Robo4 that is required for interaction with paxillin a series of GST-Robo4 fusion proteins spanning the entire length of the cytoplasmic tail were created (FIG. 4A). In vitro binding assays with purified recombinant paxillin demonstrated that the amino terminal half of the Robo4 tail (494-731) is necessary and sufficient for direct interaction with paxillin (FIG. 4B). Four additional GST-Robo4 fusion proteins encompassing approximately 70 amino acid fragments of the amino terminal half of the cytoplasmic tail were then generated (FIG. 4C). In vitro binding assays revealed that paxillin selectively interacts with a fragment of the Robo4 tail residing between the CC0 and CC2 motifs (604-674; FIG. 4D). To determine whether this region of Robo4 was necessary for interaction with paxillin amino acids 604-674 were deleted from the cytoplasmic tail and this mutant GST-Robo4 fusion protein subjected to in vitro binding assays. While interaction with paxillin was attenuated, so was interaction with a known Robo4-binding protein, Mena, indicating that elimination of amino acids 604-674 affects the conformation of the Robo4 tail. To circumvent this issue, smaller deletions were created within this 70 amino acid stretch and additional in vitro binding assays performed. Using this approach a mutant GST-Robo4 fusion protein was identified lacking 36 amino acids (604-639; FIG. 9) that lost binding to paxillin, but retained binding to Mena (FIG. 4E). This region of Robo4 is heretofore referred to as the paxillin interaction motif (PIM).

### Example 6

The Paxillin Interaction Motif is required for Robo4-dependent Inhibition of Haptotaxis: It was next determined whether the paxillin interaction motif of Robo4 is important for functional activity of the receptor. A mutant form of full length Robo4 lacking amino acids 604-639 (Robo4ΔPIM) was generated by site directed mutagenesis and used in haptotaxis migration assays. Robo4ΔPIM failed to mediate Slit2-directed inhibition of migration towards a gradient of fibronectin (FIG. 4F), demonstrating that the region of the Robo4 tail necessary for paxillin binding is likewise required for Robo4-dependent inhibition of protrusive activity.

### Example 7

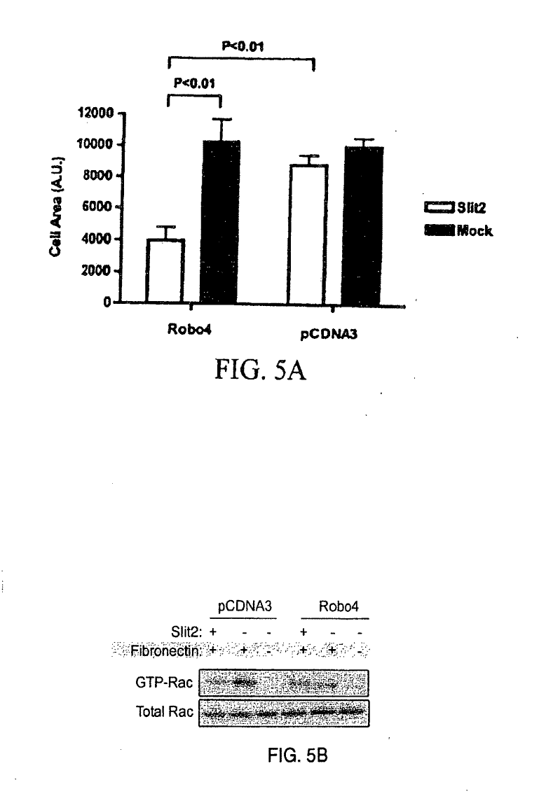

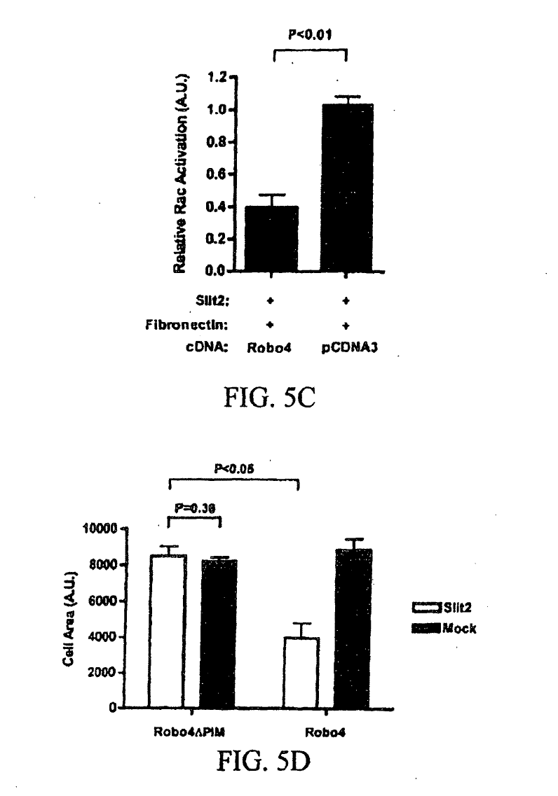

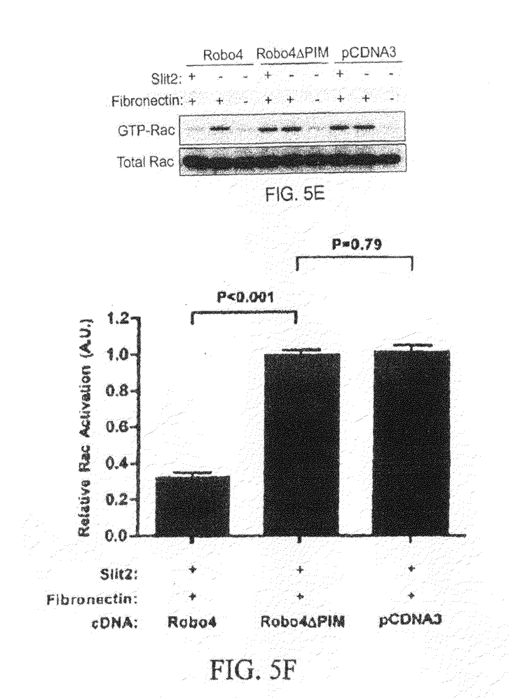

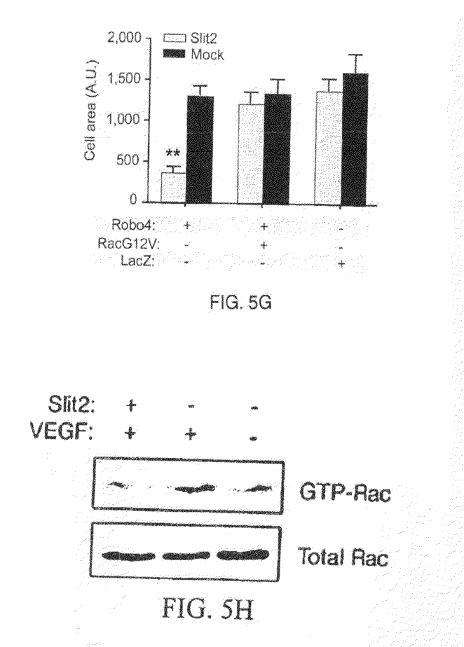

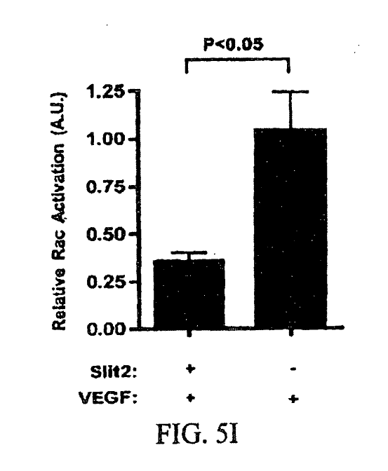

Slit2-Robo4 Signaling Inhibits Cell Spreading and Rac and ARF6: The ability of immobilized Slit2 to inhibit the migration of cells expressing Robo4 on fibronectin could potentially result from negative regulation of adhesion and/or spreading on this ECM protein. To determine whether Slit2-Robo4 signaling influences these processes, HEK 293 cells were transfected with Robo4 or empty vector (pcDNA3) and subjected to adhesion and spreading assays on fibronectin. Although cells expressing Robo4 adhered normally to coverslips coated with fibronectin and Slit2 (Slit2N (SEQ ID NO: 7)), they were significantly less spread than cells transfected with pcDNA3 (FIG. 5A). These data indicate that Slit2-Robo4 signaling modulates intracellular pathways that control cell spreading.

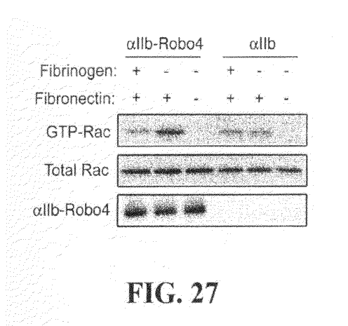

The ability of a cell to spread on an ECM protein, such as fibronectin, is regulated by activation of the Rho family of small GTPases, which include Rho, Cdc42 and Rac migration (Nobes and Hall, 1995; Nobes and Hall, 1998). Of these proteins, Rac plays an essential role in promoting the actin polymerization that leads to cell spreading and migration (Nobes and Hall, 1995; Nobes and Hall, 1998). This established relationship between Rac and cell spreading indicated that Slit2-Robo4 signaling might inhibit adhesion-dependent activation of Rac. To evaluate this, HEK 293 cells were transfected with Robo4 or pcDNA3, plated onto dishes coated with fibronectin and Slit2 (Slit2N (SEQ ID NO: 7)) and Rac-GTP levels were assayed using GST-PBD pull down assays. Additionally, cells expressing αIIb:β3 or αIIb-Robo4:β3 were plated on fibronectin and fibrinogen, and Rac-GTP levels were analyzed. Cells expressing Robo4 or αIIb-Robo4:β3 exhibited significantly less adhesion-stimulated Rac activation when compared to cells transfected with pcDNA3 or αIIb:β3 (FIGS. 5B, 5C and FIG. 27). We repeated these experiments with the Robo4ΔPIM, and found that cells expressing this mutant receptor were refractory to Slit2 (FIG. 5E).

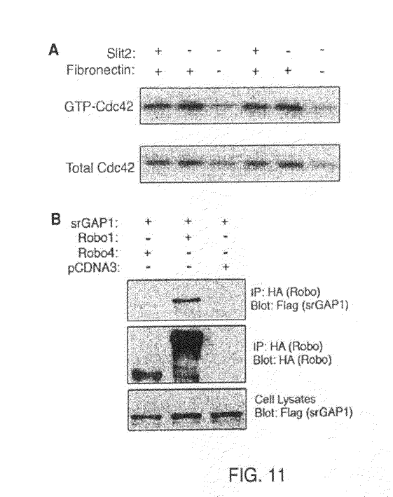

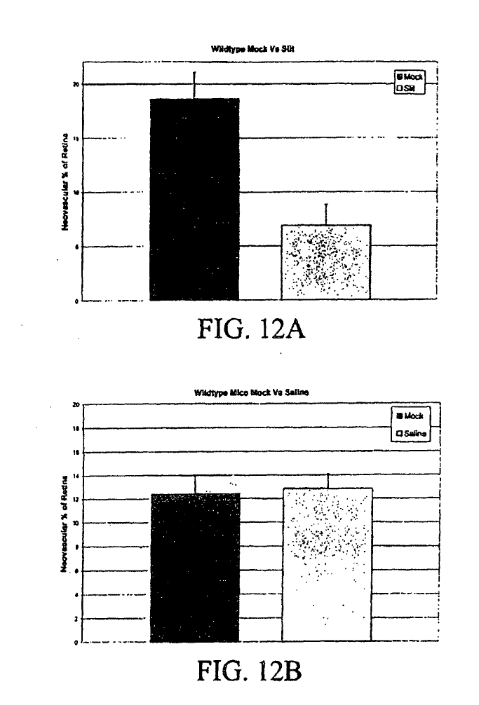

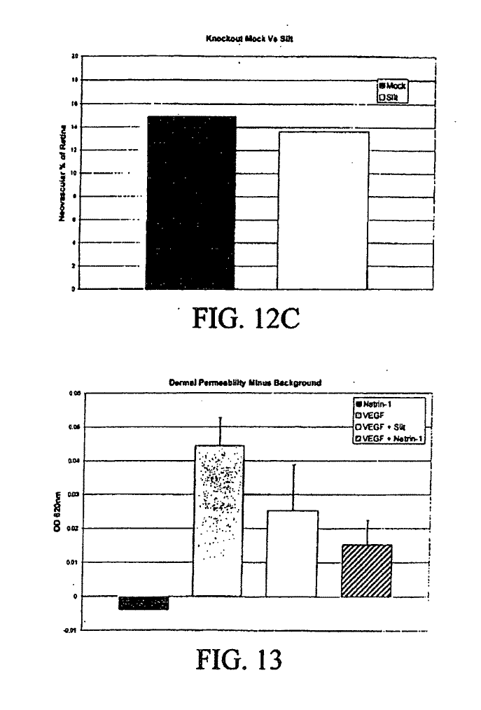

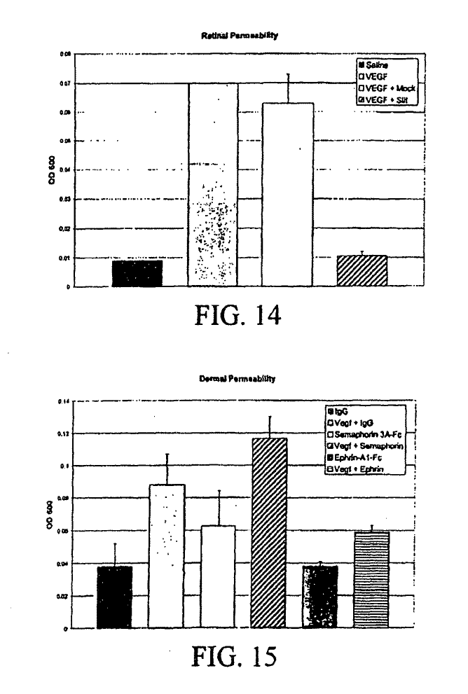

Cdc42 activation was also examined in cells expressing Robo4. We found that Cdc42 activation in cells expressing Robo4 was unaltered by exposure to Slit2 (FIG. 11A). This result is supported by the observation that Robo4 does not interact with the Robo1 binding-protein srGAP1, a known GTPase activating protein for Cdc42 (FIG. 11B). Together, these data demonstrate that Slit2-Robo4 signaling specifically inhibits adhesion-induced activation of Rac.

To confirm that Robo4-dependent inhibition of cell spreading was due principally to suppression of Rac activation, we co-transfected HEK 293 cells with Robo4 and a dominant active form of Rac, Rac (G12V), and subjected them to spreading assays. Cells expressing Rac (G12V) were refractory to Robo4-dependent inhibition of cell spreading (FIG. 5G), demonstrating that Slit2-Robo4 signaling blocks protrusive activity by inhibiting Rac.

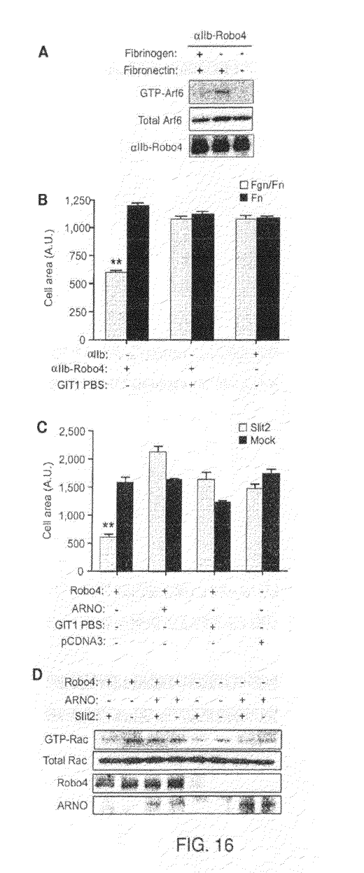

Our finding that Robo4 interacts with paxillin and inhibits protrusive activity prompted us to determine whether Robo4 signaling impinges upon the ARF6 pathway. Cells expressing αIIb-Robo4:β3 were plated on fibronectin alone, or fibronectin and fibrinogen, and ARF6-GTP levels were analyzed using a GST-GGA3 affinity precipitation technique. While fibronectin stimulated activation of ARF6, fibrinogen reduced Arf6-GTP levels in cells expressing αIIb-Robo4:β3 (FIG. 16A). This result demonstrated that Robo4 signaling inhibits ARF6 activation and suggested that Robo4's ability to block Rac activity stems from its regulation of ARF6.

### Example 8

The Paxillin Interaction Motif is required for Robo4-dependent Inhibition of Cell Spreading and Rac Activation: Whether Robo4ΔPIM was competent to inhibit fibronectin-induced cell spreading and Rac activation was next evaluated. HEK 293 cells were transfected with Robo4ΔPIM, plated onto fibronectin and Slit2 coated surfaces and subjected to spreading or Rac assays. This mutant form of the receptor was incapable of inhibiting cell spreading and adhesion-dependent Rac activation (FIGS. 5D, E and F), demonstrating that the paxillin interaction motif is essential for functional activity of Robo4 in vitro.

To confirm that Robo4-dependent inhibition of cell spreading was due principally to suppression of Rac activation, HEK 293 cells were co-transfected with Robo4 and a dominant active form of Rac, Rac (G12V), and subjected to spreading assays. Cells expressing Rac (G12V) were refractory to Robo4-dependent inhibition of cell spreading (FIG. 50), demonstrating that Slit2-Robo4 signaling blocks spreading by inhibiting Rac activity.

### Example 9

Slit2 Inhibits VEGF-induced Rac Activation in Primary Human Endothelial Cells: Slit2 inhibits VEGF-stimulated migration of several primary human endothelial cell lines (Park et al., 2003), and Rac plays an essential role for in VEGF-induced cell motility (Soga et al., 2001a; Soga et al., 2001b). It was therefore determined whether Slit2-Robo4 signaling could inhibit Rac activation in an endogenous setting. Human Umbilical Vein Endothelial Cells (HUVEC) were stimulated with VEGF in the presence and absence of Slit2 (Slit2N (SEQ ID NO: 7)), and GTP-Rac levels were analyzed using GST-PBD pull down assays. Slit2 treatment completely suppressed VEGF-stimulated Rac activation (FIGS. 5H and I), demonstrating that endogenous Slit2-Robo4 signaling modulates Rac activation.

### Example 10

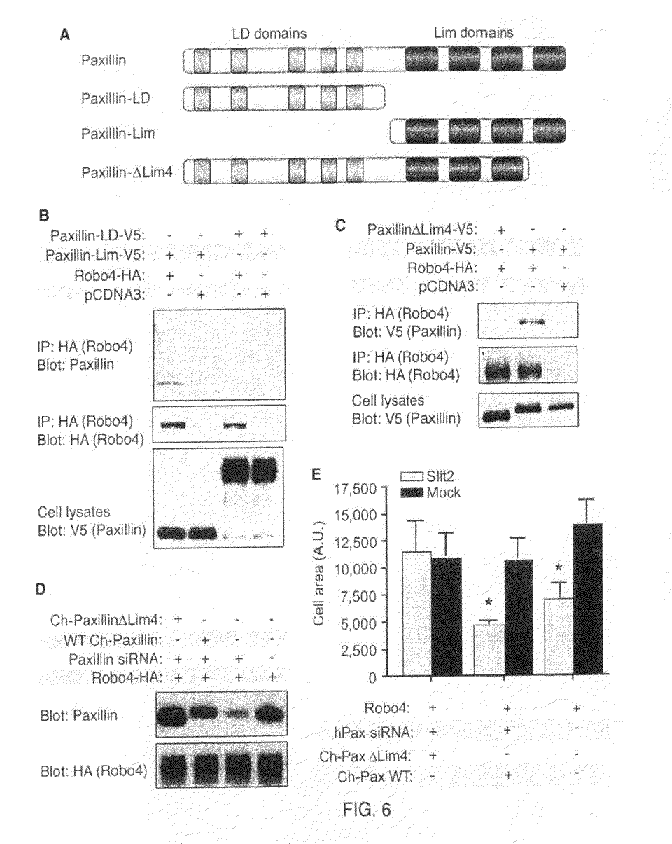

Lim4 of Paxillin is required for Interaction with Robo4 and Robo4-dependent Inhibition of Cell Spreading: Although Robo4ΔPIM maintains its interaction with Mena (FIG. 4E), it is possible that this mutation perturbed interaction of Robo4 with proteins other than paxillin. To address this issue definitively, paxillin mutants were generated that disrupt association with Robo4. Paxillin is a modular protein composed of N-terminal leucine/aspartic acid (LD) repeats and C-terminal Lim domains (FIG. 6A). Analysis of the clones recovered from the yeast two-hybrid screen (see FIG. 9A) indicated that the Lim domains, particularly Lim3 and Lim4, are important for interaction with Robo4. To validate this notion, co-immunoprecipitation experiments were performed using HEK 293 cells co-transfected with the Robo4 tail and either paxillin-LD or paxillin-Lim. Paxillin-Lim, but not paxillin-LD was found in Robo4 immunoprecipitates (FIG. 6B), demonstrating that the Lim domains of paxillin are necessary and sufficient for interaction with Robo4. To clarify which Lim domain is required for binding to Robo4, serial deletions were made from the carboxy terminus of paxillin, cotransfected with the Robo4 tail into HEK 293 cells, and coimmunoprecipitation experiments performed. Deletion of the Lim4 domain of paxillin completely abrogated binding to Robo4 (FIG. 6C), demonstrating that this region of paxillin is critical for its ability to interact with Robo4.

Delineation of the Robo4 binding site on paxillin allowed direct evaluation of the role of paxillin in Robo4-dependent inhibition of cell spreading. Endogenous paxillin was knocked-down in HEK 293 cells using siRNA and reconstituted with wild type chicken paxillin (Ch-paxillin) or Ch-paxillin ΔLim4 (FIG. 6D). These cells were then subjected to spreading assays on coverslips coated with fibronectin and Slit2 (Slit2N (SEQ ID NO: 7)). Cells expressing Ch-paxillin ΔLim4 were refractory to Robo4-dependent inhibition of cell spreading, while cells expressing Ch-paxillin exhibited the characteristic reduction in cell area (FIG. 6E). These data confirm that interaction of paxillin with the Robo4 enables Slit2-Robo4 signaling to suppress cell spreading.

### Example 11

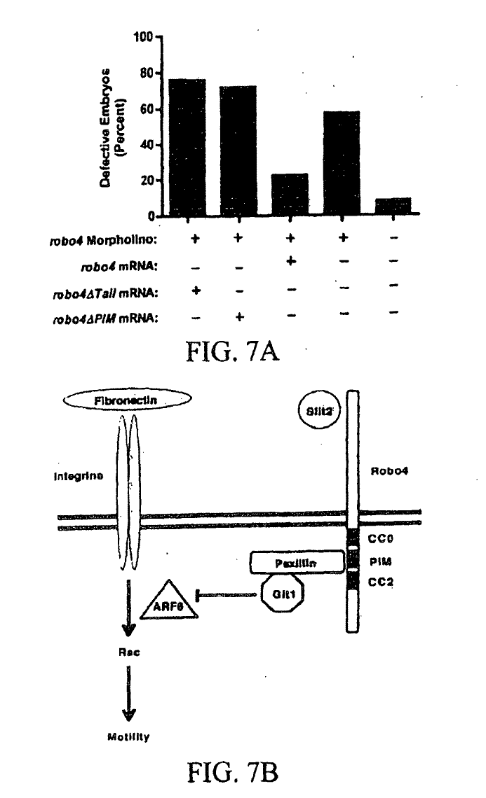

The Paxillin Interaction Motif is required for Vascular Guidance in vivo: The requirement of the paxillin interaction motif of Robo4 during zebrafish vascular development was assessed. As described previously, injection of robo4 MO into TG (fli1:egfp)^{yl} embryos caused disorganization of the intersegmental vessels (see FIG. 1B). Co-injection of robo4ΔPIM RNA exacerbated the defects caused by the robo4 MO, while wild-type robo4 RNA suppressed these defects (FIG. 7A). The inability of both robo4Δtail and robo4ΔPIM RNA to rescue vascular patterning defects in morphant embryos demonstrates that the 36 amino acid paxillin interaction motif is a critical signal transduction module in the Robo4 cytoplasmic tail. Further, these data indicate that the interaction between paxillin and Robo4 is essential for proper patterning of the zebrafish vasculature.

### Example 12

Robo4 blocks Rac-dependent protrusive activity through inhibition of ARF6: Our determination that Robo4 interacts with paxillin and inhibits protrusive activity prompted us to determine whether Robo4 impinges upon the ARF6 pathway. Cells expressing αIIb-Robo4:β3 were plated on fibronectin alone, or fibronectin and fibrinogen, and ARF6-GTP levels were analyzed using a GST-GGA3 affinity precipitation technique. While fibronectin stimulated activation of ARF6, fibrinogen reduced ARF6-GTP levels in cells expressing αIIb-Robo4:β3 (FIG. 16A). This result demonstrated that Robo4 signaling inhibits ARF6 activation and suggested that Robo4's ability to block Rac activity stems from its regulation of ARF6.

Next we analyzed the requirement of a paxillin-GIT1 complex in Robo4-dependent inhibition of protrusive activity. The paxillin binding sequence (PBS) on GIT1 is found at the carboxy-terminus of the protein and has been shown to prevent interaction of GIT1 and paxillin (Uemura et al., 2006). Cells were transfected with αIIb-Robo4:β3 and either an empty vector or the GIT1-PBS and subjected to spreading assays on fibronectin or fibronectin and fibrinogen. As described previously, cells expressing αIIb-Robo4:β3 displayed a decrease in cell area when plated on fibrinogen, but this was lost in cells transfected with the GIT1-PBS (FIG. 16B). We repeated this experiment in cells expressing full length Robo4 plated on fibronectin or fibronectin and Slit2 (Slit2N (SEQ ID NO: 7)), and similar to the chimeric receptor experiment, the GIT1-PBS prevented the Slit2-dependent decrease in cell area (FIG. 16C). These data demonstrate that a functional paxillin-GIT1 complex is required for Slit2-Robo4 signaling.

To determine whether Slit2-Robo4 signaling inhibits protrusive activity by inactivating ARF6, we co-expressed the ARF6 guanine nucleotide exchange factor ARNO with Robo4 and performed spreading assays. Overexpression of ARNO blocked the ability of Slit2 to reduce cell area, indicating that a principal effect of Slit2-Robo4 signaling is to prevent GTP-loading of ARF6 (FIG. 16C). If ARNO restored the ability of Robo4-expressing cells to spread on Slit2, we reasoned that it should likewise re-establish Rac activation in response to fibronectin. Indeed, overexpression of ARNO led to normal levels of GTP-Rac in cells plated on fibronectin and Slit2 (FIG. 16D). Together these experiments demonstrate that Slit2-Robo4 signaling inactivates ARF6, which leads to the local blockade of Rac activation and the subsequent inhibition of the membrane protrusion necessary for cell spreading and migration.

### Example 13

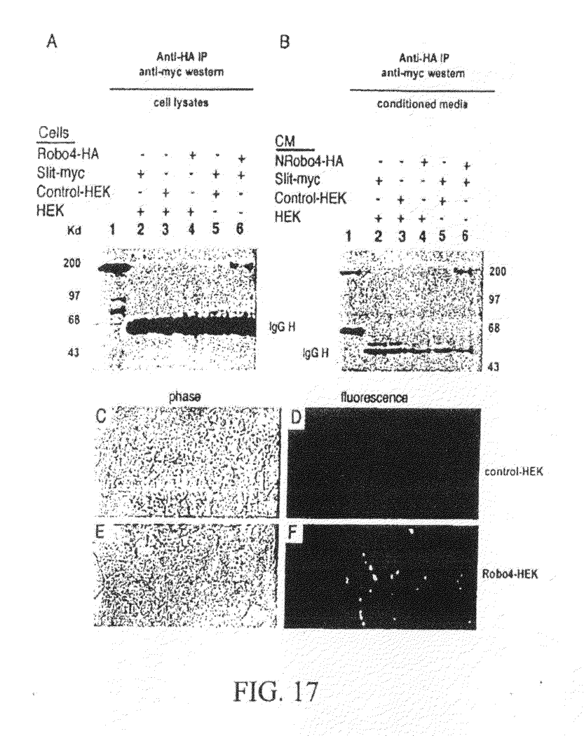

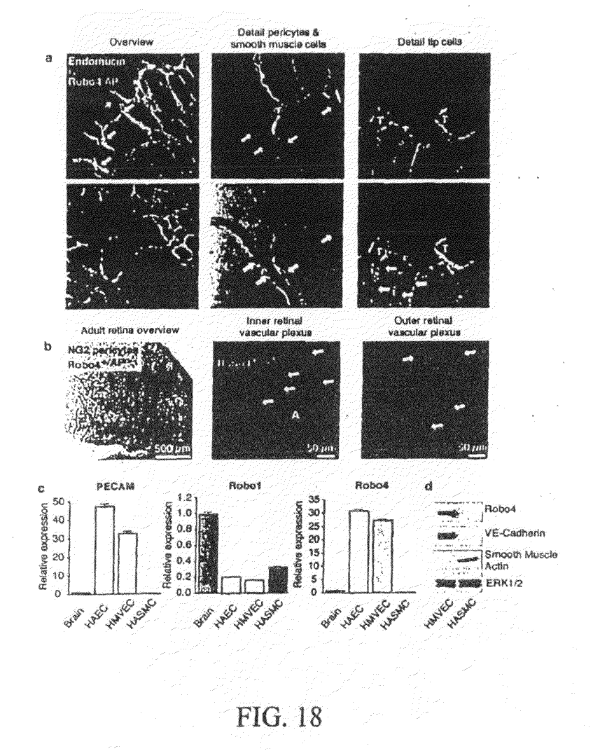

Immunoprecipitation Demonstrates Interaction Between Slit Ligand and Robo4 Receptor: Cell lysates from untransfected human embryonic kidney cells (HEK), HEK cells transfected with Slit tagged with a myc epitope (Slit-myc), HEK cells transfected with Robo4 tagged with a HA epitope (Robo4-HA) and HEK cells transfected with a control vector (Control-HEK) were immunoprecipitated. Slit-myc protein was detected by Western blot with an anti-myc antibody after Slit-myc and Robo4-HA cell lysates were combined and immunoprecipitated with an anti-HA antibody (FIG. 17A, lane 6). The specificity of this interaction was confirmed by the absence of detectable Slit protein with all other combinations of lysates (FIG. 17A, lanes 2-5). The same amount of lysate was used in each experiment. A Western blot analysis of the Slit-myc cell lysates served as a control and demonstrated that the Slit protein has a mass of approximately 210 kD in accordance with previous reports (FIG. 17A, lane 1). The lower bands shown in lanes 2-6 of FIG. 17A correspond to immunoglobulin heavy chains.

Conditioned media from untransfected HEK cells (HEK CM), HEK cells transfected with Slit tagged with a myc epitope (Slit-myc CM), HEK cells transfected with the N-terminal soluble ectodomain of Robo4 tagged with the HA epitope (NRobo4-HA CM) and HEK cells transfected with control vector (Control-HEK CM) was also immunoprecipitated. The full-length Slit-myc protein (210 KD) and its C-terminal proteolytic fragment (70 KD) were detected in Slit-myc CM by an anti-myc antibody (FIG. 17B, lane 1). Slit-myc protein was also detected by Western blot after Slit-myc and Robo4-HA conditioned media were combined and immunoprecipitated with an anti-HA antibody (FIG. 17B, lane 6). The specificity of this interaction was confirmed by the absence of Slit protein with all other combinations of conditioned media.

As is shown in FIG. 17C through FIG. 17F, Slit protein binds to the plasma membrane of cells expressing Robo4. Binding of Slit-myc protein was detected using an anti-myc antibody and an Alexa 594 conjugated anti-mouse antibody. As can be seen in FIG. 17D and FIG. 17F, binding was detected on the surface of Robo4-HEK cells (FIG. 17F) but not Control-HEK cells (FIG. 17D).

### Example 14

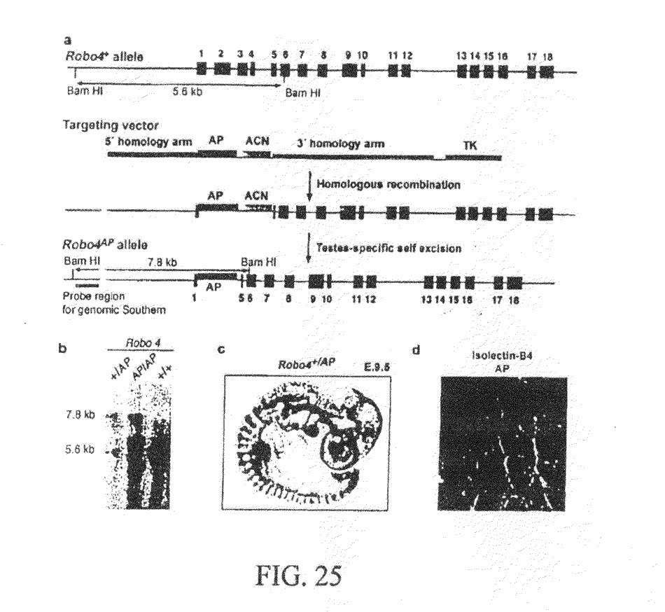

Robo4 Knockout Mouse: To ascertain the functional significance of Robo4 in vivo, knockout mice were produced using standard techniques. To produce the knockout mice, exons one through five of the gene expressing Robo4 were replaced with an alkaline phosphatase (AP) reporter gene using homologous recombination. This allele, Robo4^{AP}, lacked the exons encoding the immunoglobulin (IgG) repeats of the Robo4 ectodomain, which are predicted to be required for interaction with Slit proteins. The Robo4^{+/AP} animals were intercrossed to generate mice that were homozygous for the targeted allele. An illustration of the genomic structure of the mice is provided in FIG. 25. Robo4^{AP/AP} animals were viable and fertile, and exhibited normal patterning of the vascular system. These data indicate that Robo4 is not required for sprouting angiogenesis in the developing mouse, and point to an alternate function for Robo4 signaling in the mammalian endothelium. Alkaline phosphatase activity was detected in these animals throughout the endothelium of all vascular beds in the developing embryos and in the adult mice, which confirmed that the Robo4^{AP} allele is a valid marker of Robo4 expression.

### Example 15

Robo4 Activation Stabilizes Mature Vessels: The central region of the murine retinal vascular plexus, comprised specifically of stalk cells, is an example of the differentiated/stabilized phenotype characteristic of a mature, lumenized vascular tube. We reasoned, therefore, that Robo4 expression in the stalk might maintain this phenotype by inhibiting processes that are stimulated by pro-angiogenic factors, such as VEGF-A. The effect of Robo4 signaling on processes stimulated by VEGF-A was evaluated using a VEGF-A endothelial cell migration assay and a VEGF-A tube formation assay. Both such assays are routinely used to investigate angiogenesis in vitro.

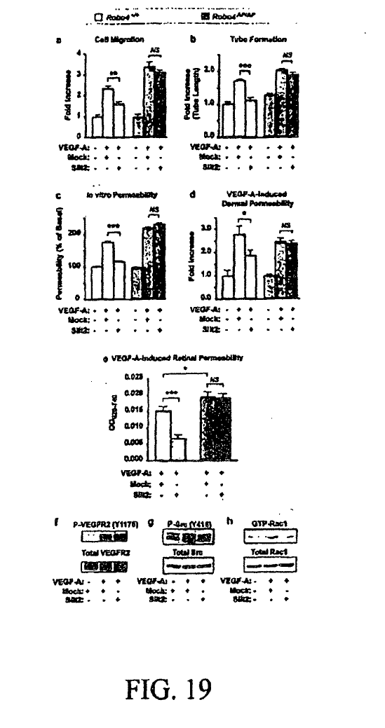

In order to conduct the endothelial cell migration and tube formation assays, endothelial cells from the lungs of Robo4^{+/+} and Robo4^{AP/AP} mice were isolated and their identity confirmed using immunocytochemistry and flow cytometry. These cells were then utilized in VEGF-A-dependent endothelial cell migration and tube formation assays. The Slit2 molecule used in these assays was Slit2N (SEQ ID NO: 7). As is shown in FIG. 19A and FIG. 19B, Slit2 inhibited both migration and tube formation of Robo4^{+/+} endothelial cells. However, the inhibitory activity of Slit2 was lost in Robo4^{AP/AP} endothelial cells. These results demonstrate that Slit2 inhibits endothelial cell migration and tube formation in a Robo4-dependent manner, and indicate that activation of Robo4 by Slit2 serves to stabilize the vascular endothelium of mature vessels.

### Example 16

Robo4 Activation Preserves Endothelial Barrier Function: In a mature vascular bed, endothelial cells do not behave independently of one another; rather they form a monolayer that prevents the movement of protein, fluid and cells from the endothelial lumen into the surrounding tissue. This barrier function was modeled in vitro using a Transwell assay to analyze the transport of horseradish peroxidase (HRP), across confluent cell monolayers of endothelial cells taken from the lungs of Robo4^{+/+} and Robo4^{AP/AP} mice. Stimulation of Robo4^{+/+} and Robo4^{AP/AP} endothelial cells with VEGF-A, a known permeability-inducing factor, enhanced the accumulation of HRP in the lower chamber of the Transwell. As is shown in FIG. 19C, however, pre-treatment of the cell monolayers with a Slit2 protein (Slit2N (SEQ ID NO: 7)) prevented this effect in Robo4^{+/+}, but not Robo4^{AP/AP} endothelial cells.

Next, the influence of Slit2 on endothelial barrier function in vivo was evaluated. A Miles assay was performed by injecting Evans Blue into the tail vein of Robo4^{+/+} and Robo4^{AP/AP} mice. VEGF-A in the absence and presence of a Slit2 protein (Slit2N (SEQ ID NO: 7)) was subsequently injected into the dermis. Analogous to the in vitro assay, VEGF-A-stimulated leak of Evans Blue into the dermis could be prevented by concomitant administration of Slit2 protein in Robo4^{+/+}, but not in Robo4^{AP/AP} mice (shown in FIG. 19D). These observations were extended by evaluating the ability of Slit2 to suppress VEGF-A induced hyperpermeability of the retinal endothelium. In particular, it was found that intravitreal injection VEGF-A in Robo4^{+/+} mice induced leak of Evans Blue from retinal blood vessels. However, such VEGF-A induced leak of Evans Blue from the retinal blood vessels was suppressed in Robo4^{+/+} mice by co-injection of the Slit2 protein Slit2N (SEQ ID NO: 39) (FIG. 19E). This experiment was repeated in retinas of Robo4^{AP/AP} mice, and it was found that Robo4^{AP/AP} were refractory to treatment with Slit2N (SEQ ID NO: 39). These data demonstrate that Robo4 mediates Slit2-dependent inhibition of VEGF-A-induced endothelial hyperpermeability in vitro and in vivo.

### Example 17

Robo4 Blocks VEGF Signaling Downstream of the VEGF Receptor: The ability of VEGF-A to promote angiogenesis and permeability is dependent upon activation of VEGFR2, which occurs by autophosphorylation following ligand binding. Subsequently, a number of non-receptor tyrosine kinases, serine/threonine kinases and small GTPases are activated to execute VEGF-A signaling in a spatially and temporally specific manner. To determine where Slit2-Robo4 signaling intersects the VEGF-A-VEGFR2 pathway, VEGFR2 phosphorylation following stimulation with VEGF-A and Slit2 was analyzed using Slit2N (SEQ ID NO: 7). Slit2N (SEQ ID NO: 7) had no effect on VEGF-A-induced VEGFR2 phosphorylation (FIG. 19F), indicating that the Slit2-Robo4 pathway must intersect VEGF-A signaling downstream of the receptor. Attention was then focused on the Src family of non-receptor tyrosine kinases, Fyn Yes and Src, due to their well-documented role in mediating VEGF-A-induced angiogenesis and permeability (Eliceiri et al., 2002; Eliceiri et al., 1999). Treatment of endothelial cells with Slit2N (SEQ ID NO: 7) reduced VEGF-A-stimulated phosphorylation of c-Src (FIG. 19G). Recently, several reports have shown that Src-dependent activation of the Rho family small GTPase, Rac1, is essential for VEGF-A-induced endothelial cell migration and permeability (Gavard et al., 2006; Garrett et al., 2007). Treatment of endothelial cell monolayers with Slit2N (SEQ ID NO: 7) prevented VEGF-A-dependent Rac1 activation (FIG. 19H). These biochemical experiments indicate that the Slit2-Robo4 pathway suppresses VEGF-A-induced endothelial migration and hyperpermeability via inhibition of an Src-Rac1 signaling axis.

### Example 18

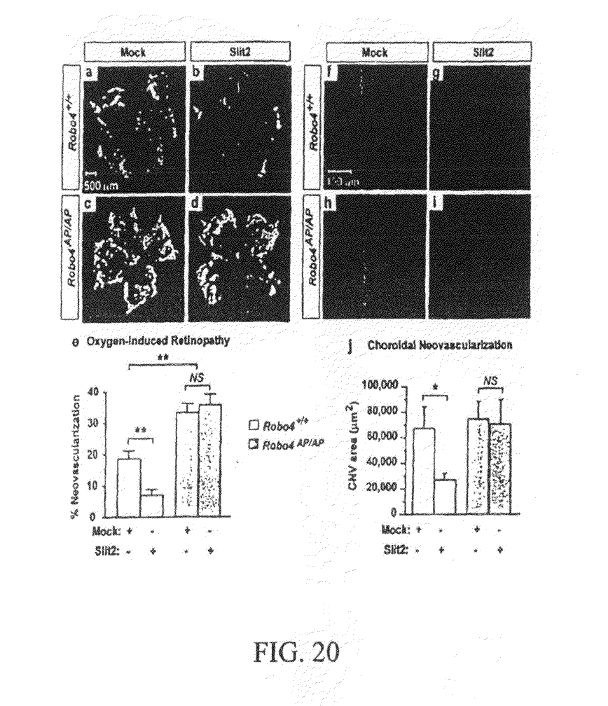

Activation of Robo4 Reduces Vascular Leak and Pathologic Angiogenesis in CNV and OIR Models: A murine model of oxygen-induced retinopathy (OIR) that mimics the ischemia-induced angiogenesis observed in both diabetic retinopathy and retinopathy of prematurity was used to investigate the effect of Robo4 signaling on retinal vascular disease. In this model, P7 mice were maintained in a 75% oxygen environment for five days and then returned to 25% oxygen for an additional five days. The perceived oxygen deficit initiates a rapid increase in VEGF-A expression in the retina, leading to pathological angiogenesis (Ozaki et al., 2000; Werdich et al., 2004. Robo4^{+/+} mice and Robo4^{AP/AP} mice were evaluated using this model. Intravitreal administration of Slit2N (SEQ ID NO: 7). markedly reduced angiogenesis in Robo4^{+/+} mice, but not in Robo4^{AP/AP} mice (FIG. 20A-FIG. 20E, where arrows indicate areas of pathological angiogenesis). Furthermore, Robo4^{AP/AP} mice displayed more aggressive angiogenesis than Robo4^{+/+} mice following exposure to hyperoxic conditions (See, e.g., FIGS. 20A and 20C).

In addition to the described OIR model, laser-induced choroidal neovascularization, which mimics age-related macular degeneration, is commonly used to study pathological angiogenesis in the mouse (Lima et al., 2005). In this model, a laser is used to disrupt Bruch's membrane, which allows the underlying choroidal vasculature to penetrate into the subretinal pigment epithelium. To discern the effect of Robo4 signaling on this pathological process, 8-12 week old Robo4^{+/+} and Robo4^{AP/AP} mice were subjected to laser-induced choroidal neovascularization followed by intravitreal injection of Slit2N (SEQ ID NO: 7). Similar to the results achieved in the mouse model of oxygen-induced retinopathy, intravitreal administration of Slit2N reduced angiogenesis in Robo4^{+/+} mice, but not in Robo4^{AP/AP} mice (See FIG. 20F-FIG. 20J). Together, the oxygen-induced retinopathy and choroidal neovascularization models indicate that two vascular beds with distinct characteristics, one a tight blood-brain barrier and the other a fenestrated endothelium, are protected from pathological insult by activation of Slit2-Robo4 signaling.

### Example 19

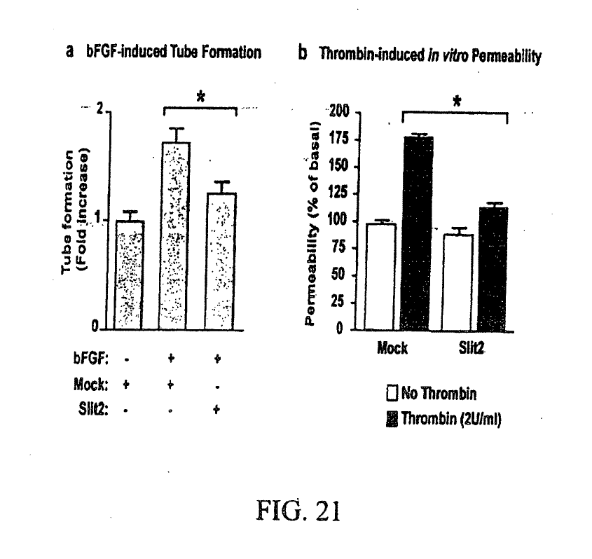

Robo4 Inhibits Signaling From Multiple Factors That Destabilize the Mature Vessel: The effect of Robo4 activation by a Slit2 molecule on the activity of bFGF, and angiogenic factor, and thrombin, the endothelial permeability factor, was evaluated. As shown in FIG. 21, Slit2N (SEQ ID NO: 7) blocked bFGF-induced endothelial tube formation and thrombin-induced permeability. These studies demonstrate that Slit-Robo4 signaling is capable of inhibiting the signaling induced by multiple angiogenic and permeability factors and support the concept that the Slit-Robo4 pathway protects the mature vascular beds from multiple angiogenic, permeability and cytokine factors.

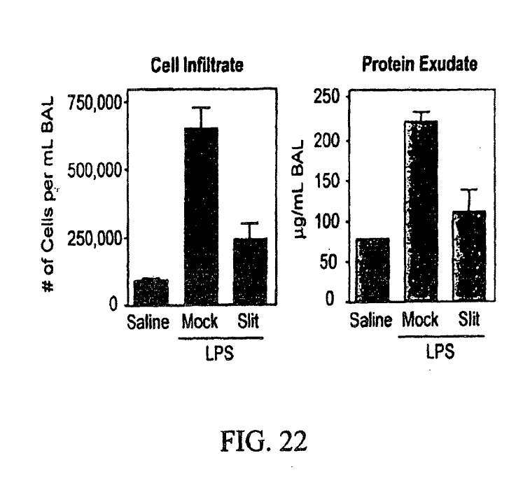

To reinforce that Robo4 signalizing protects vasculature from multiple angiogenic, permeability and cytokine factors, the effect of Robo4 activation by Slit2N (SEQ ID NO: 7) was evaluated in a mouse model of acute lung injury. In this model, the bacterial endotoxin LPS was dosed to the mice via intratracheal administration. Exposure to the bacterial endotoxin leads to a cytokine storm that causes catastrophic destabilization of the pulmonary vascular bed and results in non-cardiogenic pulmonary edema (Matthay et al., 2005). Following intratracheal administration of LPS, the mice were treated with Slit2N (SEQ ID NO: 7) or Mock preparation, which was a sham protein extract that served as a control. As shown in FIG. 22, the concentrations of inflammatory cells and protein in bronchoalveolar lavages (BAL) from mice treated with Slit2N (SEQ ID NO: 7) were significantly lower than in the mice treated with the Mock preparation. These results demonstrate that activating Robo4 under these circumstances provides potent vascular stabilization and suggest that Slit2-Robo4 is a potent vascular stabilization pathway that works to preserve the integrity of the mature endothelium and maintain vascular homeostasis against an extreme form of cytokine storm.

### Example 20

Administration of Slit2 Protein Reduces Mortality in Mouse Model of Avian Flu: In the following example, the effect of Slit protein on the survival of mice infected with Avian Flu Virus was analyzed. A total of 120 female BALB/c mice were inoculated intranasally with 50 μl of a 1:400 dilution of the Avian Flu Virus, strain H5N1/Duck/Mn/1525/81. The mice used in this example were obtained from Charles River and had an average weight ranging from 18-20 grams. With reference to Table 2, the mice were randomly divided into 6 cages of 20 mice each, and each group were subjected to daily treatments for 5 days. Survivorship (death) and body weight were observed during and after treatment.

TABLE 2

| # mice/ | | Infected | | | |
|---|---|---|---|---|---|
| Cage | Group # | y or n | Compound | Dosage | Treatment Schedule |
| 20 | 1 | Y | PSS | 50 μl volume | Qd X 4 or 5 (5 if |
| | | | | | possible) beg −4 before |
| | | | | | virus exposure, I.V. |
| 20 | 2 | Y | SLIT “Mock” 1 | 15.625 μl | Same as # 1 |
| | | | | SLIT/Mock + | |
| | | | | 34.375 μl PSS per | |
| | | | | mouse | |
| 20 | 3 | Y | SLIT “Mock” 2 | 1.5625 μl | Same as # 1 |
| | | | | SLIT/Mock + 48.44 μl | |
| | | | | PSS per mouse | |
| 20 | 4 | Y | SLIT - Conc. 1 | 15.625 μl of 800 μg/ml | Same as # 1 |
| | | | | SLIT + | |
| | | | | 34.375 μl PSS per | |
| | | | | mouse | |
| 20 | 5 | Y | SLIT - Conc. 2 | 1.5625 μl of 800 μg/ml | Same as # 1 |
| | | | | SLIT + 48.44 μl | |
| | | | | PSS per mouse | |
| 20 | 6 | Y | Ribavirin | 75 mg/kg/day | 0.1 ml I.P. BID X 5 |
| | | | | | days |

Briefly, as shown in Table 2, Group 1 was treated with physiological saline solution (PSS) a negative control. Groups 2 and 3 were treated with a Mock preparation. Groups 4 and 5 were treated with different concentrations of a Slit protein (Slit2N (SEQ ID NO: 7)). As a positive control, the 20 mice of group 6 were treated with intraperitoneally with 75 mg/kg/day of Ribavirin brought up in a total volume of 0.1 mL PSS.

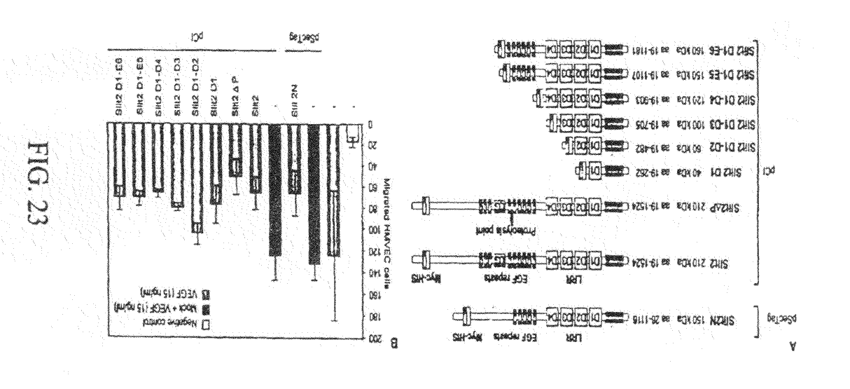

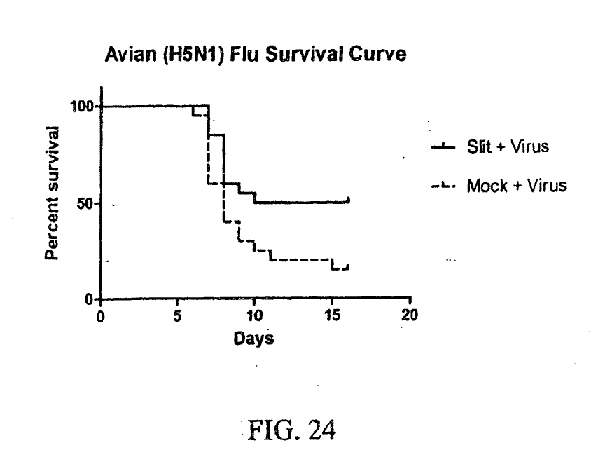

The results of the analysis are illustrated in FIG. 24 and detailed in Table 3. After 23 days, the mice treated with Slit protein in Groups 4 and 5 had a lower mortality than those mice that did not receive Slit protein in Groups 1, 2, and 3. The Group 4 mice, treated with 12.5 μg of Slit per dose, had a 25% survivability rate. The Group 5 mice, treated with 1.25 μg of Slit per dose, had a 50% survivability rate. In contrast to the survivorship of Groups 4 and 5, only 5% (1/20) of the negative control mice in Group 1, treated with PSS, survived past 23 days.

Table 3 shows that at 14 days after inoculation, the average body weights of the survivors in Groups 1, 2, and 3 were significantly lower than the Slit treated survivors in Groups 4 and 5. Moreover, 10/20 mice in Group 5, which was the lower of the Slit treatment concentrations, survived with body weights averaging 17.6 grams at 21 days, nearly as high as the starting average body weight of 17.7 grams. Therefore, those infected mice treated with Slit protein were able to maintain their body weights better than the untreated mice.

TABLE 3

| | | Day | | | | | | | | | |
|---|---|---|---|---|---|---|---|---|---|---|---|
| | | 0 | 1 | 2 | 3 | 4 | 5 | 6 | 7 | 8 | 9 |
| Cage | Alive | 20 | 20 | 20 | 20 | 20 | 19 | 17 | 11 | 8 | 3 |
| #1 | Total | 20 | 20 | 20 | 20 | 20 | 20 | 20 | 20 | 20 | 20 |
| | Av. | 17.6 | | | | | | | | | |
| | Wt. | | | | | | | | | | |
| Cage | Alive | 20 | 20 | 20 | 20 | 20 | 20 | 19 | 14 | 7 | 3 |
| #2 | Total | 20 | 20 | 20 | 20 | 20 | 20 | 20 | 20 | 20 | 20 |
| | Av. | 17.6 | | | | | | | | | |
| | Wt. | | | | | | | | | | |
| Cage | Alive | 20 | 20 | 20 | 20 | 20 | 20 | 19 | 12 | 8 | 6 |
| #3 | Total | 20 | 20 | 20 | 20 | 20 | 20 | 20 | 20 | 20 | 20 |
| | Av. Wt. | 17.6 | | | | | | | | | |
| Cage | Alive | 20 | 20 | 20 | 20 | 20 | 20 | 17 | 13 | 10 | 7 |
| #4 | Total | 20 | 20 | 20 | 20 | 20 | 20 | 20 | 20 | 20 | 20 |
| | Av. Wt. | 17.4 | | | | | | | | | |
| Cage | Alive | 20 | 20 | 20 | 20 | 20 | 20 | 20 | 17 | 12 | 11 |
| #5 | Total | 20 | 20 | 20 | 20 | 20 | 20 | 20 | 20 | 20 | 20 |
| | Av. Wt. | 17.7 | | | | | | | | | |
| Cage | Alive | 20 | 20 | 20 | 20 | 20 | 20 | 20 | 20 | 20 | 20 |
| #6 | Total | 20 | 20 | 20 | 20 | 20 | 20 | 20 | 20 | 20 | 20 |
| | Av. | 17.5 | | | | | | | | | |
| | Wt. | | | | | | | | | | |

| | | 10 | 11 | 12 | 13 | 14 | 15 | 16 | 17 | 18 | 19 | 20 | 21 | 22 | 23 |
|---|---|---|---|---|---|---|---|---|---|---|---|---|---|---|---|
| Cage | Alive | 2 | 2 | 1 | 1 | 1 | 1 | 1 | 1 | 1 | 1 | 1 | 1 | 1 | 1 |
| # 1 | Total | 20 | 20 | 20 | 20 | 20 | 20 | 20 | 20 | 20 | 20 | 20 | 20 | 20 | 20 |
| | Av. | | | | | 12.5 | | | | | | | 16.0 | | |
| | Wt. | | | | | | | | | | | | | | |
| Cage | Alive | 2 | 2 | 2 | 2 | 2 | 2 | 2 | 2 | 2 | 2 | 2 | 2 | 2 | 2 |
| # 2 | Total | 20 | 20 | 20 | 20 | 20 | 20 | 20 | 20 | 20 | 20 | 20 | 20 | 20 | 20 |
| | Av. | | | | | 12.5 | | | | | | | 15.3 | | |
| | Wt. | | | | | | | | | | | | | | |
| Cage | Alive | 5 | 4 | 4 | 4 | 4 | 3 | 3 | 3 | 3 | 3 | 3 | 3 | 3 | 3 |
| # 3 | Total | 20 | 20 | 20 | 20 | 20 | 20 | 20 | 20 | 20 | 20 | 20 | 20 | 20 | 20 |
| | Av. | | | | | 13.0 | | | | | | | 16.1 | | |
| | Wt. | | | | | | | | | | | | | | |
| Cage | Alive | 6 | 5 | 5 | 5 | 5 | 5 | 5 | 5 | 5 | 5 | 5 | 5 | 5 | 5 |
| # 4 | Total | 20 | 20 | 20 | 20 | 20 | 20 | 20 | 20 | 20 | 20 | 20 | 20 | 20 | 20 |
| | Av. | | | | | 16.0 | | | | | | | 18.5 | | |
| | Wt. | | | | | | | | | | | | | | |
| Cage | Alive | 10 | 10 | 10 | 10 | 10 | 10 | 10 | 10 | 10 | 10 | 10 | 10 | 10 | 10 |
| # 5 | Total | 20 | 20 | 20 | 20 | 20 | 20 | 20 | 20 | 20 | 20 | 20 | 20 | 20 | 20 |
| | Av. | | | | | 15.4 | | | | | | | 17.6 | | |
| | Wt. | | | | | | | | | | | | | | |
| Cage | Alive | 20 | 20 | 20 | 20 | 20 | 20 | 20 | 20 | 20 | 20 | 20 | 20 | 20 | 20 |
| # 6 | Total | 20 | 20 | 20 | 20 | 20 | 20 | 20 | 20 | 20 | 20 | 20 | 20 | 20 | 20 |
| | Av. | | | | | 17.2 | | | | | | | 18.3 | | |
| | Wt. | | | | | | | | | | | | | | |

### Example 21

Fragments of Slit Proteins Work to Activate Robo4: FIG. 23 illustrates various constructs of the Slit2 protein. As has already been described herein, the 150 kD protein Slit2N (SEQ ID NO: 7), has been found to be effective in in vitro and in vivo models, including Miles assays, assays for retinal permeability, tube formation and endothelial cell migration and in OIR and CNV models of ocular disease. Moreover, as is shown in FIG. 23, the (40 kD) protein SlitD1 (SEQ ID NO: 42) and Slit2N (SEQ ID NO: 39) constructs exhibits similar activity to full length Slit2 (SEQ ID NO: 40) in a VEGF-induced endothelial cell migration assay.

### Example 22

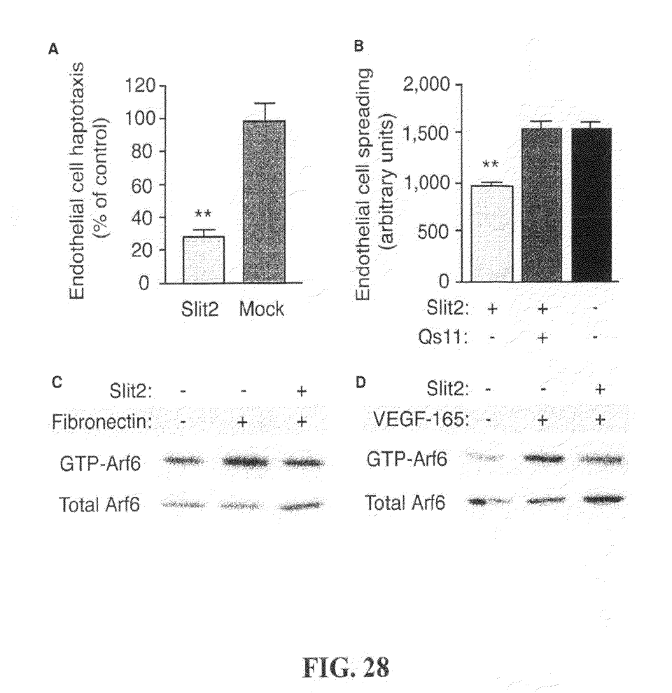

Slit2 Inhibits Cell Protrusion in Endothelial Cells via ARF-GAPs: Our experiments utilized model cell systems to decipher the signal transduction cascade downstream of Robo4. To determine whether this molecular mechanism is important for Robo4 function in primary cells we subjected human microvascular endothelial cells to haptotaxis migration assays on transwells coated on the underside with a mixture of fibronectin and Slit2 (Slit2N (SEQ ID NO: 7)). Analogous to HEK cells, Slit2 blocked fibronectin-driven cell migration (FIG. 28A). Next we performed spreading assays on coverslips coated with the 9-11 fragment of fibronectin (a ligand for alph5beta1 integrin) and Slit2 (Slit2N (SEQ ID NO: 7)) and again found that Slit2 suppressed cellular protrusive activity stimulated by integrin ligation (FIG. 28B). To analyze the role of GIT1 in Slit2-dependent inhibition of cell protrusion, we pre-treated endothelial cells with a small molecule inhibitor of Arf-GAPs, Qsl1, and then subjected them to spreading assays on 9-11 and Slit2 (Slit2N (SEQ ID NO: 7)). Arf-GAP inhibition prevented the reduction in cell area elicited by Slit2 (FIG. 28B) demonstrating that Arf-GAP activity is essential for Slit2-dependent inhibition of cell protrusion.

### Example 23

Slit2 Blocks ARF6 Activation in Response to Fibronectin and VEGF-165: These cell biological data suggested that Slit2-Robo4 signaling in endothelial cells should block ARF6 activation in response to integrin ligation. To confirm this idea, we plated endothelial cells onto dishes coated with fibronectin and Slit2, and ARF6-GTP levels were analyzed using the GST-GGA3 affinity precipitation technique. Consistent with results from CHO cells (FIG. 16A), Slit2 (Slit2N (SEQ ID NO: 7)) blocked the fibronectin-induced increase in ARF6-GTP (FIG. 28C). In addition to fibronectin, the angiogenic and permeability-inducing factor VEGF-165, which exists in vivo as an extracellular matrix bound form, has been suggested to activate ARF6. To clarify the effect of VEGF-165 and Slit2 (Slit2N (SEQ ID NO: 7)) on ARF6 activity we plated endothelial cells on dishes coated with both proteins, and ARF6-GTP levels were analyzed using the GST-GGA3 affinity precipitation technique. VEGF-165 activated ARF6 and Slit2 prevented this activation (FIG. 28D) demonstrating that Slit2 inhibits both extracellular matrix protein- and growth factor-induced ARF6 activation.

### Example 24

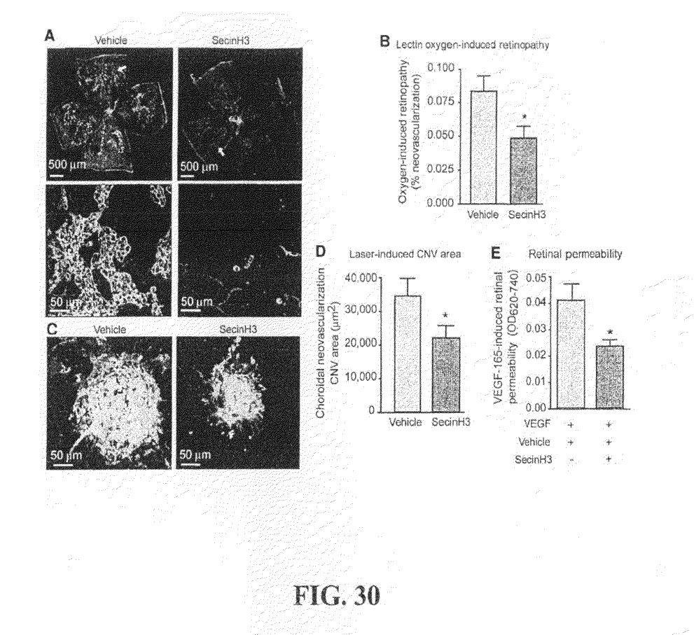

Inhibition of ARF6 Prevents Pathologic Angiogenesis and Vascular Leak: Robo4 mediates Slit2-dependent inhibition of neovascular tuft formation and endothelial hyperpermeability (REF), processes that are initiated and perpetuated by extracellular matrix proteins, such as fibronectin, and growth factors, such as VEGF (REFs). The involvement of ARF6 in integrin and VEGF receptor signaling, and the ability of Slit2 to block ARF6 activation in response to fibronectin and VEGF-165 led us to speculate that ARF6 might be a critical nexus in the signaling pathways regulating pathologic angiogenesis and vascular leak. To test this hypothesis, we injected a small molecule inhibitor of Cytohesin Arf-GEFs, SecinH3 (FIG. 29), into the eyes of wild-type mice and subjected these animals to oxygen-induced retinopathy (OIR), laser-induced choroidal neovascularization (CNV) and VEGF-165-induced retinal permeability assays. SecinH3, but not a DMSO control inhibited neovascular tuft formation in OIR (FIG. 30A and FIG. 30B) and CNV (FIG. 30C and FIG. 30D), and retinal hyperpermeability caused by VEGF-165 (FIG. 30E), thus demonstrating the central involvement of Arf-GTPases in these pathological processes and demonstrating that blockade of Arf activation is a potential therapy for diseases characterized by pathologic angiogenesis and vascular leak.

### Example 25

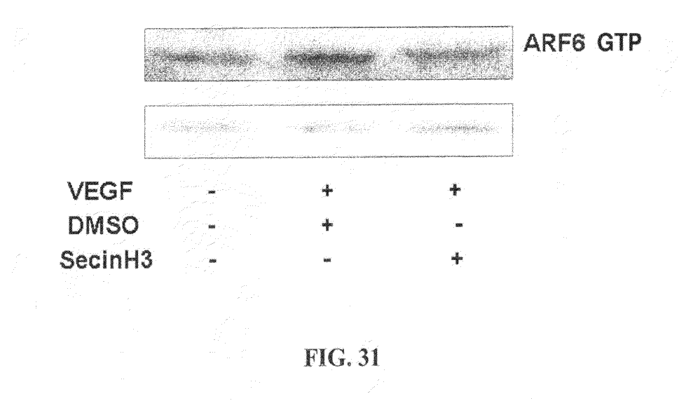

Secin-H3 Inhibits VEGF Induced ARF6-GTP: To test whether Secin-H3 attenuated the accumulation of ARF6-GTP, human microvessel endothelial cells (HMVEC) were either not treated (FIG. 31 Leftmost lane), treated with 20 ng/mlVEGF\ DMSO (FIG. 31 Middle lane) or treated with 20 ng/ml VEGF\ 50 μM Secin-H3 (FIG. 31 Rightmost lane). Cell lysates were probed with an ARF6-GTP antibody or an ARF6 antibody; relative amounts of these ARF6 species were compared via a western blot. The cells were then washed twice with ice-cold PBS and lysed in 50 mM Tris pH 7.0, 500 mM NaCl, 1 mM MgCl₂, 1 mM EGTA, 1 mM DTT, 0.5% NP-40, 1× protease inhibitors, 1× phosphatase inhibitors and 50 μg/ml GST-GGA3-VHS-GAT. The lysate was centrifuged for 5 min at 14,000 rpm and the supernatant was incubated with 50 μl of glutathione agarose for 30 min at 4° C. Following three washes with lysis buffer, bound proteins were eluted with 2× sample buffer. Arf6 was detected by western blotting with Arf6-specific antibodies The results of these experiments demonstrate that the small organic molecule Secin-H3 blocks the accumulation of ARF6-GTP (compare FIG. 31, top panel middle lane and top panel right lane).

### Example 26

Secin-H3 Inhibits VEGF Induced Migration of HMVECs: A cell migration assay was performed using a modified Boyden chamber Transwell assay to test whether Secin-H3 (FIG. 29) can inhibit VEGF induced HMVEC migration in an in vitro assay. Cells were plated as described herein, and subjected to either a control treatment or an experimental treatment. The control treatment comprised 0.2% BSA and DMSO and the experimental treatment comprised 0.2% BSA+15 ng/ml VEGF-165 and DMSO+15 μM Secin-H3. 50 μl of 0.2% BSA, the experimental treatment comprised 0.2% BSA\15 ng/ml VEGF-165, 0.2% BSA\15 ng/ml VEGF-165\DMSO, and 0.2% BSA\15 ng/ml VEGF-165\15 μM Secin-H3 were plated into each well of a 48-well Boyden chamber apparatus (NeuroProbe, Cabin John, Md.), and the wells were overlayed with an 8 μm pore polycarbonate membrane (NeuroProbe) that had been previously coated with 50 μg/ml human fibronectin (Biomedical Technologies, Inc., Stoughton, Mass.). Then 3.75×10⁴ cells human microvessel endothelial cells (HMVEC) were added to the upper chambers, and the migration was allowed to proceed for 3 h at 37° C. (5% CO,). The membranes were then removed, fixed in methanol, stained with a Hema 3 stain set (Fisher Scientific, Pittsburgh, Pa.), and placed (migrated-side down) onto 50×75 mm glass slides. Cells present on the migrated side of the membrane were manually counted (three random 200× fields per well), and data points for each experiment represent the average number of migrated cells from six separate wells (three 200× fields counted per well).

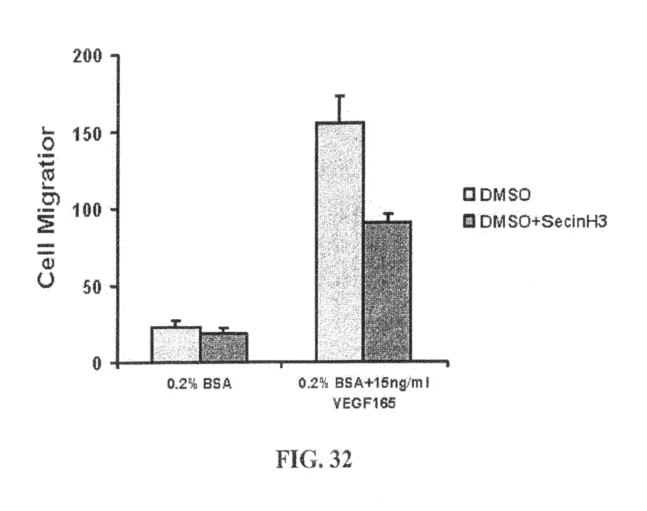

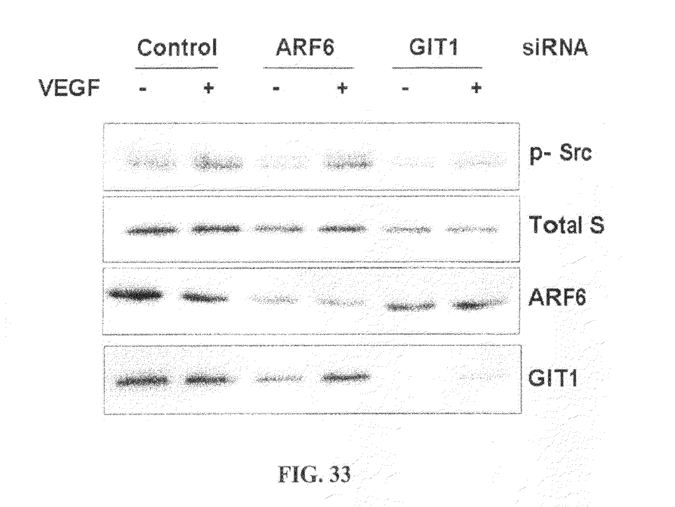

Results depicted in FIG. 32 show that cells treated with VEGF-165 demonstrate a cell migration response, which is not attenuated by further treatment with DMSO. Treatment with Secin-H3 attenuated the VEGF-165 induced cell migration response.

### Example 27

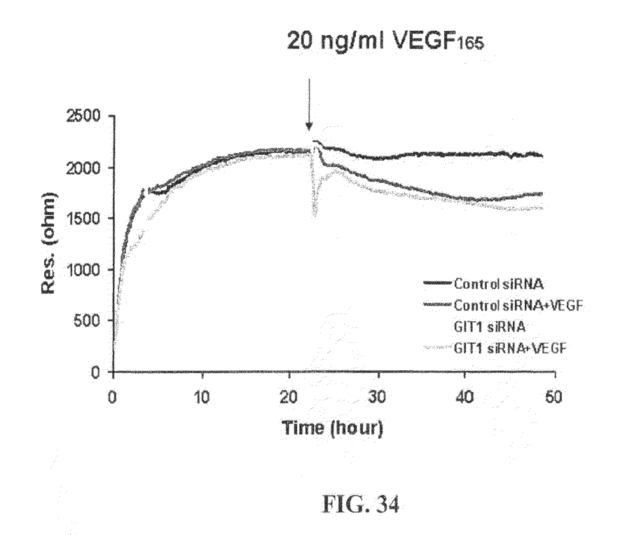

GIT1 RNAi Increases VEGF Induced HMVEC Permeability: FIG. 34 illustrates the results from HMVEC permeability assays in which the question was tested as to whether a reduction in expression of GIT1 via RNAi could enhance VEGF induced permeability. Cells were plated as described herein and transfected with either a control siRNA or a GIT1 siRNA. Each siRNA group was split and half of the cells were treated with VEGF-165. As depicted in FIG. 34, VEGF induced permeability was enhanced in the GIT1 siRNA cells compared to the other cells.

### Example 28

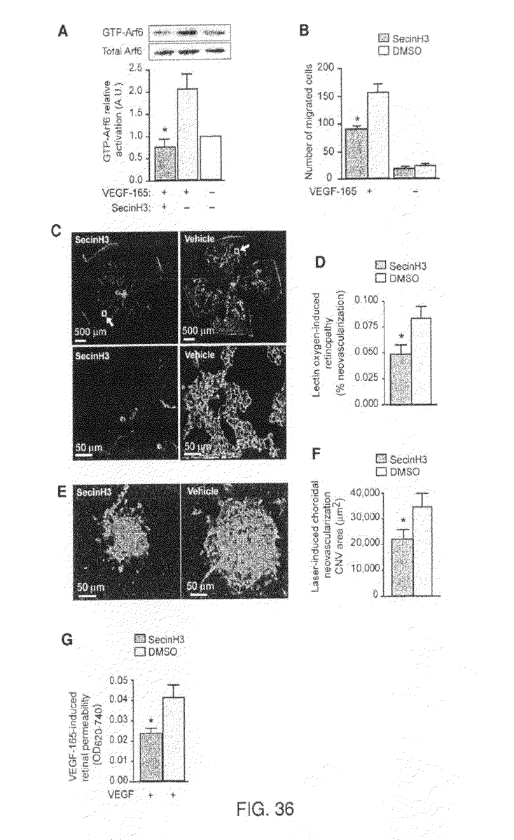

Secin-H3 Inhibits Arf6 activation, VEGF Induced Migration of Endothelial Cells, Neovascular Tuft Formation in Models of OIR and CNV, and Retinal Hyperpermeability Caused by VEGF-165: We determined the effect of SecinH3 on VEGF signaling by pre-treating endothelial cells with the inhibitor or vehicle control and performing Arf6 activation and cell migration assays. SecinH3 prevented both VEGF-induced Arf6 activation and VEGF-induced cell migration (FIG. 36 A, B). Next, we injected SecinH3 into the eyes of wild-type mice and subjected these animals to oxygen-induced retinopathy (OIR), laser-induced choroidal neovascularization (CNV) and VEGF-165-induced retinal permeability assays. SecinH3, but not a vehicle control of DMSO inhibited neovascular tuft formation in OIR (FIG. 36 C, D) and CNV (FIG. 36 E, F), and retinal hyperpermeability caused by VEGF-165 (FIG. 36 G), thus demonstrating the central involvement of Arf-GTPases in these pathological processes.

Oxygen-induced retinopathy: Briefly, P7 pups along with nursing mothers were placed in 80% oxygen, which was maintained by Pro-OX oxygen controller (BioSpherix). Pups were removed on P12 and given an intraocular injection of SecinH3 at a final concentration of 21.6 μM. Mice were sacrificed on P17, eyes enucleated and fixed for 2 hours in 4% paraformaldehyde. Retinas were then dissected and stained overnight using Alexa Fluor 488 conjugated isolectin 1:50 (Invitrogen). Retinal flatmounts were generated and images taken using Axiovert 200 fluorescence microscopy (Carl Zeiss). Neovascularization was quantified using AxioVision software (Carl Zeiss). Data are presented as mean±s.e. for 14 wild-type mice.

Laser-induced choroidal neovascularization: Briefly, two-three month old mice were anesthetized with Avertin (2-2-2 Tribromoethanol, 0.4 mg/g; Acros Organics) and the pupils dilated with 1% tropicamide (Alcon). An Iridex OcuLight GL 532 nm laser photocoagulator (Iridex) with slit lamp delivery system was used to create three burns 3 disc diameters from the optic disc at 3, 6, and 9 o'clock with the following parameters: 150 mW power, 75 μm spot size, and 0.1 second duration. Production of a bubble at the time of laser indicating rupture of Bruch's membrane is an important factor in obtaining CNV; therefore, only burns in which a bubble was produced were included in this study. Immediately after laser treatment and 3 days later, mice were given an intravitreal injection of SecinH3 at a final concentration of 21.6 μM. One week after laser treatment, mice were sacrificed and choroidal flat mounts generated. Alexa 488 conjugated isolectin (Sigma) was used to stain CNV. Flat mounts were examined using a Zeiss LSM 510 confocal microscope (Carl Zeiss) and CNV quantified using ImageJ software (NIH). Data are presented as mean±s.e. for at least 15 wild-type mice.

Retinal Permeability: Retinal permeability was assessed as previously described²⁴. Briefly, 8-10 week old mice were anesthetized with Avertin (2-2-2 Tribromoethanol, 0.4 mg/g; Acros Organics). Mice were given an intraocular injection of 1.5 μL of 35.7 μg/mL VEGF-165 (R&D Systems Inc) and either 216 μM of SecinH3 in 2% DMSO (we estimated the final concentration to be 216 μM and DMSO to be 0.2%) or 2% DMSO alone. Six hours later, 50 μL of 60 mg/mL Evans Blue solution was administered via the tail vein. After two hours, mice were sacrificed and perfused with citrate-buffered formaldehyde to remove intravenous Evans Blue. Eyes were enucleated and retinas dissected. Evans Blue dye was eluted in 0.4 mL formamide for 18 hours at 70° C. The extract was ultra-centrifuged through a 5 kD filter for 2 hours. Absorbance was measured at 620 nm. Background absorbance was measured at 740 nm and subtracted out. Data are presented as mean±s.e. for six wild-type mice.

### Example 29

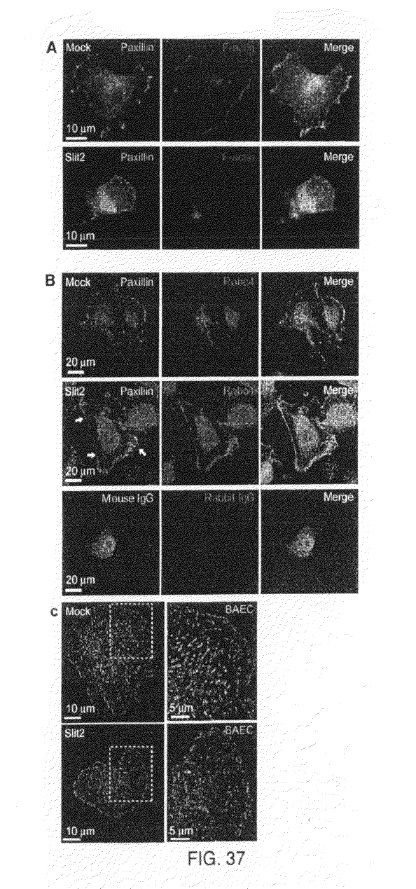

Slit2 blocks recruitment of paxillin to focal adhesions: To assess the effect of Slit ligation of Robo4 on the subcellular distribution of paxillin, cells were permitted to adhere to cover slips coated with fibronectin in the presence or absence of Slit2, and stained for endogenous paxillin. In the absence of Slit2 (Mock), HEK cells expressing full length Robo4 spread normally and formed abundant focal adhesions near the cell periphery that stained for paxillin (FIG. 37A, top panel). In contrast, cells plated on Slit2 (Slit2N (SEQ ID NO: 7)) and fibronectin exhibited reduced spreading, stained significantly less for F-actin, and formed much fewer and smaller paxillin-stained focal adhesions (FIG. 37A, bottom panel). Control HEK cells (not expressing Robo4) adhered on Fibronectin and Slit2 exhibited no differences in morphology when compared with adhesion on Fibronectin alone (data not shown), indicating the effect of Slit is dependent on Robo4.

We repeated the assay with bovine aortic endothelial (BAE) cells, which endogenously express Robo4. On substrata coated with fibronectin and Slit2 (Slit2N (SEQ ID NO: 7)), BAE cells exhibited reduced spreading compared to cells adhered to fibronectin alone (Mock), indicating a similar inhibitory effect of Slit2-Robo4 signaling (FIG. 37B). BAE cells adhered to fibronectin and Slit2 formed small paxillin-stained structures different from the mature focal adhesions of fibronectin-adherent cells that were larger and elongated (FIG. 37B). The inhibitory effect of Slit2 on cell spreading appears to be transient, as cells adhered for longer periods of time, with or without Slit2, exhibited similar degrees of spreading and focal adhesion formation (data not shown). Together with the observation that Slit2 induces recruitment of paxillin to Robo4, we propose that Robo4 ligation reduces the availability of paxillin for recruitment to adhesive structures, thereby contributing to reduced cell spreading and migration.

### Example 30

Slit2 recruits paxillin to the cell surface: Our data suggest that in Robo4-expressing cells, Slit2 treatment redistributes paxillin from focal adhesions to the cell surface, where it co-localizes with the receptor. To determine the veracity of this notion, we analyzed the subcellular distribution of paxillin and Robo4 in cells incubated in the absence and presence of a Slit2 protein (Slit2N (SEQ ID NO: 7)). Because Slit2 blocks cell spreading, and thus prevents clear visualization of the plasma membrane, we performed these experiments in endothelial cells pre-spread on fibronectin. In cells treated with Mock, paxillin was found almost exclusively in focal adhesions, while Robo4 was localized to the cell surface (FIG. 37C, top panel). In cells treated with Slit2, however, a significant portion of paxillin appeared at the cell surface and co-localized with Robo4; this alteration in localization was coincident with a reduction of paxillin in focal adhesions (FIG. 37C, bottom panel). These data reveal that Slit2 stimulation of Robo4 redistributes paxillin from focal adhesion to the cell surface, where it is accessible to Robo4.

### Example 31

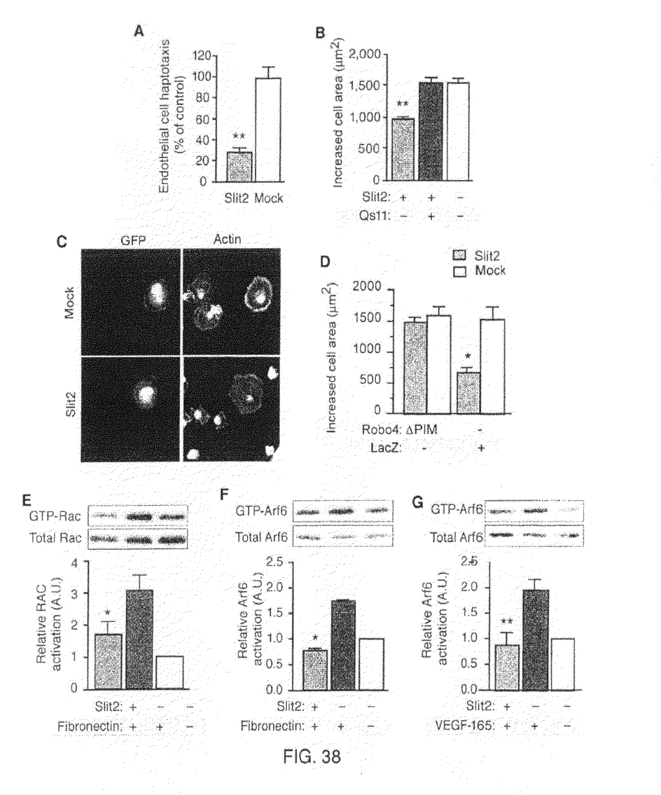

The Paxillin Interaction Motif (PIM) is required for Slit2 signaling in Endothelial Cells: Next, we determined the requirement of paxillin binding to Robo4 for Slit2-dependent inhibition of cell spreading. We expressed Robo4ΔPIM or LacZ in endothelial cells and performed spreading assays on fibronectin, in the absence and presence of a Slit2 protein (Slit2N (SEQ ID NO: 7)). Cells expressing Robo4ΔPIM (GFP+) spread equivalently on both Mock and Slit2, while untransfected cells expressing endogenous Robo4 (GFP−) were markedly inhibited on Slit2, but not Mock (FIGS. 38 C and D). These data indicate that paxillin binding to Robo4 is necessary for Slit2 to modulate cellular protrusive activity.

### Example 32

Slit2 blocks activation of Rac and Arf6 in Endothelial Cells: These cell biological data suggested that Slit2-Robo4 signaling in endothelial cells should block Rac and Arf6 activation in response to integrin ligation. To confirm this idea, we plated endothelial cells onto dishes coated with fibronectin and a Slit2 protein (Slit2N (SEQ ID NO: 7)), and analyzed Rac-GTP and Arf6-GTP levels. Consistent with results from HEK and CHO cells, Slit2 efficiently blocked the fibronectin-induced increase in Rac-GTP (FIG. 38 E) and Arf6-GTP levels (FIG. 38 F). In addition to fibronectin, the angiogenic and permeability-inducing factor VEGF-165, exists in vivo as a component of the extracellular matrix. To ascertain the effect of VEGF-165 and Slit2 on Arf6 activity, we plated endothelial cells on dishes coated with both proteins, and analyzed Arf6-GTP levels. While VEGF-165 alone activated Arf6, addition of Slit2 prevented this activation (FIG. 37 D), demonstrating that Slit2 inhibits both extracellular matrix protein- and growth factor-induced Arf6 activation.

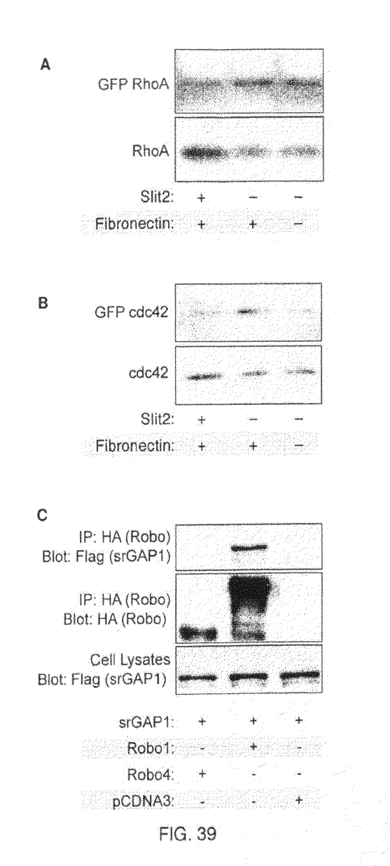

To gain insight into the regulation of Rho and Cdc42 by Slit2, we plated endothelial cells on fibronectin in the absence and presence of Slit2 (Slit2N (SEQ ID NO: 7)) and analyzed Rho-GTP and Cdc42-GTP levels. While Rho activation was unaltered by Slit2, Cdc42 activation was significantly reduced (FIG. 39). The effect of Slit2 on Cdc42 was somewhat surprising given that Robo4 does not interact with the Robo1 binding-protein srGAP1, a known GTPase activating protein for Cdc42 (FIG. 39).

### Materials and Methods

Reagents: HEK 293 and COS-7 cells, and all IMAGE clones were from ATCC. SP6 and T7 Message Machine kits were from Ambion. HUVEC, EBM-2 and bullet kits were from Cambrex. Yeast two-hybrid plasmids and reagents were from Clontech. FBS was from Hyclone. Anti-HA affinity matrix, Fugene6 and protease inhibitor cocktail were from Roche. Goat Anti-Mouse-HRP and Goat Anti-Rabbit-HRP secondary antibodies were from Jackson ImmunoResearch. Anti-V5 antibody, DAPI, DMEM, Lipofectamine 2000, Penicillin-Streptomycin, Superscript III kit, Trizol and TrypLE Express were from Invitrogen. Anti-Flag M2, Phosphatase Inhibitor Cocktails, Soybean Trypsin Inhibitor and Fatty acid-free Bovine Serum Albumin (BSA) were from Sigma. Human fibronectin was from Biomedical Technologies and Invitrogen. Costar Transwells and Amicon Ultra-15 Concentrator Columns were from Fisher. Rosetta2 E. coli were from Novagen. Glutathione-Sepharose 4B, parental pGEX-4T1 and ECL PLUS were from Amersham-Pharmacia. Coomassie Blue and PVDF were from BioRad. Quick change site-directed mutagenesis kit was from Stratagene. Normal Rat IgGagarose conjugate was from Santa Cruz. Robo4 morpholinos were from Gene Tools. Oligonucleotides for PCR were from the University of Utah Core Facility. Alexa564-Phalloidin, Anti-GFP and Goat Anti-Rabbit A1ex488 were from Molecular Probes. Low melt agarose was from NuSieve. T7 in vitro transcription/translation kit was form Promega.

Molecular Biology: The Robo4-HA, Slit2-Myc-His and chicken paxillin plasmids have been previously described (Park et al., 2003; Nishiya et al., 2005). Robo4-NH2 was amplified from Robo4-HA and cloned into EcoRV/NotI of pcDNA3-HA. Robo4-COOH was amplified from Robo4-HA by overlap-extension PCR and cloned into EcoRV/NotI of pcDNA3-HA. The amino terminal half of the human Robo4 cytoplasmic tail (AA 465-723) was amplified by PCR and cloned into (EcoRI/BamHI) of pGBKT7. Murine Robo4 fragments were amplified by PCR and cloned into BamHI/EcoRI of pGEX-4T1. Murine Hic-5, Mena and paxillin (including deletions) were amplified from IMAGE clones by PCR and cloned into EcoRV/NotI of pcDNA3-V5. GST-Robo4ΔPIM and full-length Robo4ΔPIM were generated by site-directed mutagenesis of relevant wild-type constructs using Quick Change. The integrity of all constructs was verified by sequencing at the University of Utah Core Facility.

Embryo Culture and Zebrafish Stocks: Zebrafish, Danio rerio, were maintained according to standard methods (Westerfield, 2000). Developmental staging was carried out using standard morphological features of embryos raised at 28.5° C. (Kimmel et al., 1995). The Tg (fli:EGFP)^{yl} transgenic zebrafish line used in this study was described in Lawson and Weinstein, 2002. Imaged embryos were treated with 0.2 mM 1-phenyl-2-thio-urea (PTU) after 24 hpf to prevent pigment formation.

Antisense Depletion of robo4: Antisense morpholino oligonucleotides (MO) directed against the exon 10/intron 10 splice site of robo4 (5′-tttttagcgtacctatgagcagtt-3′, SEQ ID NO:28) were dissolved in 1× Danieau's Buffer at a concentration of 5 ng/nl, respectively. Before injection, the morpholino was heated at 65° C. for 5 minutes, cooled briefly, mixed with a negligible amount of dye to monitor injection efficiency, and approximately 1 nl was injected into the streaming yolk of 1-2 cell stage embryos.

Reverse Transcription (RT) PCR: RNA was extracted from 20 uninjected and 20 robo4 MO-injected embryos using Trizol, reagent and subsequent cDNA synthesis was performed using Superscript III primed by a mixture of both random hexamers and oligo dT primers. robo4 was amplified from cDNA by PCR with a forward primer in exon 8 (5′-caacaccagacacttacgagtgcc-3′, SEQ ID NO:29) and a reverse primer in exon 12 (5′-ttcgaaggccagaattacctggc-3′, SEQ ID NO:30) using the following parameters: (94° C. for 4′, 94° C. for 30″, 58° C. for 30″, 68° C. for 45″, 68° C. for 1′). To identify the linear range of the PCR reaction, cDNA was amplified for 23, 25, 27 and 30 cycles. β-actin was amplified using a forward primer (5′-cccaaggccaacagggaaaa, SEQ ID NO:31) and a reverse primer (5% ggtgcccatctectgacaa-3′, SEQ ID NO:32) from all samples to control for cDNA input.

Whole-Mount Indirect Immunofluorescence: Briefly, age-matched 24 and 48 hpf embryos were dechorionated and fixed in 4% PFA/4% sucrose/PBS overnight at 4° C. The embryos were then washed in PBS/0.1% Tween-20, dehydrated to absolute methanol, re-hydrated back to PBS-Tween 20, further permeabilized in PBS/1% Triton-X, rinsed in PBS/1% Triton-X/2% BSA, blocked at room temperature in PBS/1% Triton-X/2% BSA /10% Sheep Serum/1% DMSO, then incubated in IgG purified anti-GFP (1:400) in blocking solution overnight at 4° C. The following day embryos were washed vigorously in PBS/1% Triton-X/2% BSA, then incubated in goat-anti-Rabbit Alexa 488 conjugated secondary antibody (1:200) in blocking solution overnight at 4° C. The following day the embryos were washed extensively in PBS/1% Triton-X/2% BSA, then embedded in 1% low melt agarose in PBS and photographed on Leica confocal microscope and processed using Adobe Photoshop software.

Cell Culture: HEK 293 and COS-7 cells were cultured in DMEM supplemented with 10% FBS and 1% penicillin/streptomycin. Human microvascular endothelial cells (HMVEC) were cultured in EGM-2 MV and human umbilical vein endothelial cells (HUVEC) were cultured in EGM-2 supplemented with 10% FBS. ECs were routinely used between passages 2 and 5.

Transfection: HEK293 and COS-7 cells were transfected with Fugene6 or Lipofectamine-2000 according to the manufacturer's protocol.

Preparation of Concentrated Slit2 Protein: COS-7 cells were transiently transfected with empty pSECTAG2 or pSECTAG2::hSlit2. Forty-eight hours later, the cells were washed twice with PBS and incubated with 6 ml salt extraction buffer (10 mM HEPES, pH 7.5, 1M NaCl and 1× protease inhibitors) for 15 minutes at 25° C. Salt extraction was repeated and the samples were centrifuged at 10,000 rpm for 20 minutes to pellet cell debris. The supernatant was loaded on Amicon Ultra-15 concentrator columns/100 kDa cutoff and centrifuged until 12 ml of salt extracts was reduced to approximately 500 μl. The concentrated protein preparations were analyzed by Coomassie Blue staining, and stored at 4° C. for up to one week. Using this protocol, Slit2 concentrations of 20-50 μg/ml were routinely obtained. In addition to preparing concentrated protein from cells transfected with Slit2 plasmid, the identical protocol was performed on cells transfected with an empty vector (pSECTAG2). This resulting preparation was referred to as a “Mock” preparation, and it was used as a control in all experiments analyzing the effect of Slit2.

Haptotaxis Migration Assay: Cells were removed from tissue culture dishes with TrypLE Express, washed once with 0.1% trypsin inhibitor, 0.2% fatty acid-free BSA in DMEM or EBM-2, and twice with 0.2% BSA in the relevant media. The washed cells were counted and resuspended at 0.3×10⁵ cells/ml. 1.5×10⁵ were loaded into the upper chamber of 12 μm Costar transwells pre-coated on the lower surface with 5 μg/ml fibronectin. The effect of Slit2 on haptotaxis was analyzed by co-coating with 0.5 μg/ml Slit2 or an equivalent amount of Mock preparation. Cell migration was allowed to proceed for 6 hours, after which cells on the upper surface of the transwell were removed with a cotton swab. The cells on the lower surface were fixed with 4% formaldehyde for 5 minutes and washed three times with PBS. For HEK 293 cells, the number of GFP-positive cells (HEK 293) on the lower surface was enumerated by counting six 10× fields on an inverted fluorescence microscope. The number of migrated cells on fibronectin/Mock-coated membranes was considered 100% for data presentation and subsequent statistical analysis.

Yeast Two Hybrid Assay: pGBKT7::hRobo4 465-723 was transformed into the yeast strain PJ694A, creating PJ694A-Robo4. A human aortic cDNA library was cloned into the prey plasmid pACT2 and then transformed into PJ694A-Robo4. Co-transformed yeast strains were plated onto SD-Leu-Trp (-LT) to analyze transformation efficiency and SD-Leu-Trp-His-Ade (-LTHA) to identify putative interacting proteins. Yeast strains competent to grow on SD-LTHA were then tested for expression of β-galactosidase by the filter lift assay. Prey plasmids were isolated from yeast strains capable of growing on SD-LTHA and expressing β-galactosidase, and sequenced at the University of Utah Core Facility.

Immunoprecipitation: Cell lysates were prepared in 50 mM Tris-Cl, pH 7.4, 50 mM NaCl, 1 mM DTT, 0.5% Triton X-100, phosphatase and protease inhibitors, centrifuged at 14K for 20 minutes to pellet insoluble material, cleared with normal IgG coupled to agarose beads for 60 minutes, and incubated for 2 hours at 4° C. with relevant antibodies coupled to agarose beads. The precipitates were washed extensively in lysis buffer and resuspended in 2× sample buffer (125 mM Tris-Cl, pH 6.8, 4% SDS, 20% Glycerol, 0.04% bromophenol blue and 1.4M 2-mercaptoethanol).

GST Pull Down Assay: Rosetta2 E. coli harboring pGEX-4T1::mRobo4 were grown to OD600 of 0.6 and induced with 0.3 mM IPTG. After 3-4 hours at 30° C., 220 rpm, the cells were lysed by sonication in 20 mM Tris-Cl pH 7.4, 1% Triton X-100, 1 μg/ml lysozyme, 1 mM DTT and protease inhibitors. The GST-fusion proteins were captured on glutathione-Sepharose 4B, washed once with lysis buffer without lysozyme and then twice with binding/wash buffer (50 mM Tris-Cl, pH 7.4, 150 mM NaCl, 1 mM DTT, 1% Triton X-100, 0.1% BSA and protease inhibitors). The GST-fusion proteins were incubated with 60 nM purified recombinant paxillin overnight at 4° C., washed extensively in binding/wash buffer, and resuspended in 2× sample buffer.

Western Blotting: Immunoprecipitates and GST-fusion proteins were incubated for 2 minutes at 100° C., separated by SDS polyacrylamide gel electrophoresis (SDS-PAGE) and transferred to a polyvinyldifluoride (PVDF) membrane. PVDF membranes were incubated with 5% nonfat dry milk in PBS+0.1% Tween20 (PBST) (PBST-M) for 60 minutes at 25° C. Blocked membranes were incubated with primary antibody (anti-Flag M2 at 1:2000; anti-HA at 1:10,000; anti-Hic-5 at 1:500; anti-paxillin at 1:10,000; anti-Rac at 1:1,000 and anti-Cdc42 at 1:500) in PBST-M for 60 minutes at 25° C., or overnight at 4° C. Membranes were washed 3×10 minutes in PBST and then incubated with secondary antibody (goat anti-mouse or goat anti-rabbit horseradish peroxidase at 1:10,000) for 60 minutes at 25° C. Membranes were washed 3×10 minutes in PBST and visualized with ECL PLUS.

In vitro Transcription/Translation: Mena-V5 was synthesized with the T7 Quick Coupled in vitro Transcription/Translation system according to the manufacturer's protocol.

Spreading Assay: Cells were plated onto coverslips coated with 5 μg/ml fibronectin or in some instances, where indicated, 30 μg/ml 9-11 fragment of fibronectin. Following a 30 minute incubation at 5% CO₂ and 37° C., the cells were washed three times with ice-cold PBS and fixed with 3.7% formaldehyde for 10 minutes at room temperature. The cells were then permeabilized with 0.2% Triton X-100 for three minutes, washed three times with PBS+0.1% Tween20 (PBST) and incubated with 10 μg/ml Rhodamine-Phalloidin for one hour at room temperature. Following three more washes in PBS-T, the coverslips were mounted in Pro-Long Gold and analyzed by confocal microscopy. The total area of at least 150 cells in three independent experiments was determined using ImageJ.

siRNA-mediated knockdown of paxillin: HEK 293 cells were transfected with 100 nM siRNA duplexes (5′-CCCUGACGAAAGAGAAGCCUAUU-3′, SEQ ID NO: 19 and 5′-UAGGCUUCUCUUUCGUCAGGGUU-3′) using LipofectAMINE 2000, according to the manufacturer's instructions. 48 h after transfection, cells were processed for biochemical analysis or cell spreading assays. Paxillin reconstitution was accomplished by transfection with an expression vector encoding chicken paxillin, which has the nucleotide sequence 5′-CCCCTACAAAAGAAAAACCAA-3′ within the siRNA target site. Knockdown and reconstitution were visualized by western blotting with paxillin antibodies and quantified by densitometry.

Rac and Cdc42 Activation Assay: Cells were detached from cell culture dishes, held in suspension for one hour in DMEM+0.2% BSA, and plated onto bacterial Petri dishes coated with 5 μg/ml fibronectin for five minutes. The cells were then washed twice with ice-cold PBS and lysed in 50 mM Tris pH 7.0, 500 mM NaCl, 1 mM MgCl₂, 1 mM EGTA, 1 mM DTT, 0.5% NP-40, 1× protease inhibitors, 1× phosphatase inhibitors and 20 μg/ml GST-PBD. The lysate was centrifuged for five minutes at 14,000 rpm and the supernatant was incubated with 30 μl of glutathione agarose for 30 minutes at 4° C. Following three washes with lysis buffer, bound proteins were eluted with 2× sample buffer. Rac and Cdc42 were detected by western blotting with antibodies specific to each protein. Rac activation levels were normalized to total Rac and the highest value in each experiment was assigned a value of 1.

Generation of Robo4^{AP/AP} mice and genotyping: The Robo4 targeting vector was electroporated into embryonic stem (ES) cells. ES cells heterozygous for the targeted allele were injected into blastocysts and then transferred to pseudopregnant females. Chimeric males were identified by the presence of agouti color and then mated to C57BL/6 females to produce ES-cell derived offspring. Genotype was confirmed by Southern blot analysis of tail DNA. Genomic DNA from ear punch or tail samples was used for PCR genotyping under the following conditions; denaturation at 94° C. for 30 seconds, annealing at 60° C. for 30 seconds, and extension at 72° C. for 60 seconds, 40 cycles. The following two primers were used for genotyping of Robo4: 5′ cccttcacagacagactctcgtatttcc 3′ (forward) and 5′cccagacctacattaccttttgccg 3′(reverse) and for AP: 5′ggcaacttccagaccattggcttg 3′(forward) and 5′ ggttaccactcccactgacttccctg 3′ (reverse).

Embryos and expression analysis: Staging of embryos, in situ hybridization, paraffin sectioning and whole-mount PECAM-1 immunohistochemistry were performed. For Northern Blot analysis, 20 μg of total RNA was loaded per lane after isolation with TRIZOL. ³²P-labelled probe was generated using prime It II Random-Primer labeling kit (Stratagene). Lung lysates were prepared with lysis buffer [1% NP-40, 150 mM NaCl, 50 mM Tris-Cl (pH 7.5), 1 mM EDTA and protease inhibitor cocktail (Roche)]. Robo4 protein from the lung lysates was detected by Western blot analysis using a polyclonal anti-Robo4 antibody as previously described.

Alkaline phosphatase (AP) staining: Embryos or tissues were fixed in 4% paraformaldehyde and 2 mM MgCl₂ in PBS overnight at 4° C. with shaking. Samples were washed three times for 15 min in PBST (PBS, 0.5% Tween 20). Endogenous alkaline phosphatase was inactivated at 65° C. for 90 min in PBS with 2 mM MgCl₂, then washed in AP buffer (100 mM Tris-Cl, pH9.5, 100 mM NaCl, 50 mM MgCl₂, 0.1% Tween 20, 2 mM Levamisole) twice for 15 minutes. Staining was carried out in BM purple substrate (Boehringer Mannheim) for embryos (Boehringer Mannheim) or NBT/BCIP for adult tissues. Staining was stopped in PBS, with 5 mM EDTA.

Whole mount immunohistochemistry after AP staining: Alkaline phosphatase (AP) staining on fixed and dissected retinas was performed as described above. Staining was stopped in PBS-5 mM EDTA. Retinas were washed twice in PBS and post-fixed 5 minutes in 4% paraformaldehyde, phosphate-buffered saline at RT, then washed twice in PBS. After 2 h hours incubation in PBlec (PBS, pH 6.8, 1% Triton-X100, 0.1 mM CaCl 0.1 mM MgCl 0.1 mM MnCl), retinas were incubated with antibodies overnight at 4° C. Pericytes were labeled using rabbit anti-NG2 antibody (1:200; Chemicon) and endothelial cells were labeled using rat anti-endomucin (Clone V.7C7 kindly provided by Dietmar Vestweber; diluted 1:20). After 3 washes in PBS-T (PBS, pH 7.4, 1% Triton-X100), samples were incubated with secondary, antibodies conjugated with the appropriate fluorochrome—Alexa Fluor 488 or 568 (Molecular Probes; Invitrogen) in PBS. After washing and a brief postfixation in 4% PFA, the retinas were flat mounted and coversliped using Mowiol/DABCO (Sigma-Aldrich) Samples were analyzed by conventional light and fluorescence microscopy using a Zeiss Stereomicroscope Stemi SV 11 Bioquad equipped with a Zeiss Axiocam HRc digital camera and by confocal laser scanning microscopy using a Zeiss LSM Meta 510. AP staining was visualized using the 633 nm HeNe laser and reflection settings. Digital images were processed using Volocity (4.0 Improvision) and compiled in Adobe Photoshop CS2.

Immunohistochemistry: Whole-mount triple immunofluorescence confocal microscopy was performed. Briefly, antibodies to PECAM, NP1, CX40, 2H3, BFABP and αSMA were used to label the limb skin of Robo4+/+ or Robo4−/− embryos at E15.5.

Binding and activity of Robo4 agonists on Robo4 expressing HEK cells: Stable cell lines expressing Robo4-HA (Robo4-HEK), or the pcDNA3 vector alone (Control-HEK), were seeded in 6-well culture dishes precoated with 100 μg/ml poly-L- lysine. Cells were incubated with HEK CM or Slit-myc CM at 37° C. After 1 hr incubation with conditioned media, followed by three washes in PBS, cells were fixed in 4% paraformaldehyde for 20 min. Cells were then washed three times with PBS and incubated with mouse anti-myc antibody (Santa Cruz Biotech) and anti-mouse Alexa 594-conjugated secondary antibody (Molecular Probes). The ability of those agonists, which bind to Robo4 to inhibit migration, was performed according to Park K W, Morrison C M, Sorensen L K, et al., “Robo4 is a vascular-specific receptor that inhibits endothelial migration,” Dev Biol 2003; 261(1):251-67.

Isolation of murine lung endothelial cells: Sheep anti-rat IgG Dynal beads (Dynal Biotech) were conjugated with either anti-PECAM-1 or anti-ICAM-2 monoclonal antibody (BD Pharmingen) at 5 μg of antibody per 1004 of beads. The beads were precoated and stored at 4° C. (4×10⁸ beads/mL of PBS with 0.1% BSA) for up to 2 weeks. The lungs from three adult mice were harvested. The lung lobes were dissected from visible bronchi and mediastinal connective tissue. The lungs were washed in 50 mL cold isolation medium (20% FBS-DMEM) to remove erythrocytes, minced with scissors and digested in 25 mL of pre-warmed Collagenase (2 mg/mL, Worthington) at 37° C. for 45 minutes with gentle agitation. The digested tissue was dissociated by triturating 12 times through a 60 cc syringe attached to a 14 gauge metal cannula and then filtered through sterile 70 μm disposable cell strainer (Falcon). The suspension was centrifuged at 400×g for 10 minutes at 4° C. The cell pellet was resuspended in 2 ml cold PBS and then incubated with PECAM-1 coated beads (15 μL/mL of cells) at room temperature for 10 minutes. A magnetic separator was used to recover the bead-bound cells, which were washed in isolation medium, and then resuspended in complete medium (EGM-2 MV, Lonza). The cells were plated in a single fibronectin-coated 75-cm² tissue culture flask and nonadherent cells were removed after overnight incubation. The adherent cells were washed with PBS and 15 ml of complete medium was added. Cultured cells were fed on alternate days with complete medium. When the cultures reached 70 to 80% confluency, they were detached with trypsin-EDTA, resuspended in 2 ml PBS and sorted for a second time using ICAM-2 conjugated beads (15 μL/mL of cells). The cells were washed and plated as above. Passages 2 to 5 were used for functional assays.

Cell Culture: Human dermal microvascular endothelial cells (HMVEC, Cambrex) were grown in EGM-2 MV, and used between passages 3 and 6.

Immunocytochemistry: 8 well chamber slides (Lab-Tek) were coated with 1.5 μg/cm² fibronectin for two hours prior to plating cells. Murine lung endothelial cells were plated overnight at 37° C. (100,000 cells/well) in complete medium, EGM-2 MV. The cells were then washed three times in PBS, and fixed in 4% paraformaldehyde for 10 minutes at room temperature. After three additional washes in PBS, the cells were washed in 1% Triton X-100 in PBS for 15 minutes at room temperature followed by three washes in PBST (0.1% Triton X-100 in PBS). The cells were then blocked in 2% BSA in PBS for 20 minutes at room temperature and incubated with primary antibody in 2% BSA: rat anti-PECAM-1 (Pharmigen), rabbit anti-Von Willebrand Factor (vWF) (DAKO) for 1 hour at room temperature. After incubation with primary antibody, the cells were washed in PBST and incubated with secondary antibody in 2% BSA: Alexa Fluor 488 donkey anti-rat IgG and Alexa Fluor 594 donkey anti-rabbit IgG (Molecular Probes) for 1 hour at room temperature. The cells were washed once in PBST, once in PBS, mounted in Vectashield mounting media (Vector Laboratories), and photographed by a confocal microscopy.

Fluorescence-Activated Cell Sorting (FACS): Murine lung endothelial cells were detached from the culture dish by brief trypsinization (no more than 2 minutes) at 37° C. Proteolysis was arrested by the addition of trypsin inhibitor in EBM-2+0.1% BSA. The cells were washed twice in FACS buffer (PBS without Ca2+ and Mg2++0.1% BSA) and then resuspended in 1 mL FACS buffer. Analysis of the expression of cell surface markers was performed with two-step immunofluorescence staining. The cells were incubated for 30 minutes at 4° C. with purified monoclonal antibodies: rat anti-PECAM-1, rabbit anti-vWF. The cells were then washed two times in FACS buffer and resuspended in 1 mL FACS buffer. The cells were then incubated for 30 minutes at 4° C. with fluorescent secondary antibody: Alexa Fluor 488 donkey anti-rat IgG and Alexa Fluor 594 donkey anti-rabbit IgG (Molecular Probes). The cells were again washed twice, resuspended in 1 mL FACS buffer and analyzed with the FACS.

Cell migration assay: Cells were labeled with CellTracker Green CMFDA (Molecular Probes) for 1 hour, washed and then starved overnight in EBM-2 supplemented with 0.1% BSA. Cells were trypsinized, washed and resuspended to 300,000 cells/mL. 100 μL of cell suspension (30,000 cells) was loaded onto 8-μm HTS FluoroBlock filters (BD Falcon) that had been previously coated on both sides with 5 μg/mL human fibronectin. Test factors were diluted in EBM-2/0.1% BSA and placed in the lower chamber. After incubation at 37° C. for 3 hours, two 5× fields from each well were photographed on an inverted fluorescence microscope (Axiovert 200). The number of migrated cells was enumerated by counting fluorescent cells. Basal migration of Robo4^{+/+} cells was set at 1. Data are presented as mean±S.E. of three independent experiments in triplicate.

Tube formation assay: Tube formation was performed as previously described⁵. In brief, lung endothelial cells isolated from Robo4^{+/+} and Robo4^{AP/AP} mice were plated onto matrigel-coated wells of a 48-well dish, and starved overnight in 0.5% serum. The cells were then stimulated with 0.48 nM VEGF-A in the absence or presence of Slit2 for 3.5 hours, and then photographed. Average tube length was determined using ImageJ software. Data are presented as mean±S.E. of three independent experiments in duplicate.

In vitro permeability assay: Lung endothelial cells (ECs) isolated from Robo4^{+/+} and Robo4^{AP/AP} mice were plated onto 3.0 μm Costar transwells pre-coated with 1.5 μg/cm² human fibronectin and grown to confluency. Cells were starved overnight, pre-treated with 0.3 nM Slit2 for 30-60 minutes and then stimulated with 2.4 nM VEGF-A for 3.5 hours. Horseradish peroxidase (HRP) was added to the top chamber at a final concentration of 100 μg/ml, and 30 minutes later the media was removed from the lower chamber. Aliquots were incubated with 0.5 mM guaiacol, 50 mM Na₂HPO₄, and 0.6 mM H₂O₂, and formation of O-phenylenediamine was determined by measure of absorbance at 470 nm. Basal permeability of monolayers was set at 100%. The data is presented as mean±S.E. of three independent experiments in triplicate.

VEGF Induced Retinal Permeability: In brief, 8-10 week old mice were anesthetized with Avertin (2-2-2 Tribromoethanol, 0.4 mg/g; Acros Organics, Morris Plains, N.J.). Mice were given an intraocular injection of 1.4 uL of 35.7 ug/mL VEGF-A (R&D Systems Inc. Minneapolis, Minn.) with 50 ng Slit2N (SEQ ID NO: 39). An injection with equivalent volume of Mock preparation was given in the contralateral eye. As indicated, other conditions of 1.4 uL of saline, Mock preparation, or slit were administered. Six hours later, mice were given an I. V. injection via the tail vein of 50 uL Evans Blue 60 mg/mL. After two hours, mice were sacrificed and perfused with citrate-buffered para-formaldehyde to remove intravenous Evans Blue. Eyes were enucleated and retinas dissected. Evans Blue dye was eluted in 0.3 mL formamide for 18 hours at 70° C. The extract was ultra-centrifuged through a 5 kD filter for 2 hours. Absorbance was measured at 620 nm. Background absorbance was measured at 740 nm and subtracted out.

Adenoviral expression of Robo4: Robo4 was expressed via adenovirus as previously described.

Miles Assay: Evans Blue was injected into the tail vein of 6-8 week old mice, and thirty minutes later either saline, or 10 ng of VEGF-A in the absence and presence of 100 ng Slit2 was injected into the dermis. After an additional thirty minutes, punch biopsies were preformed and Evans Blue was eluted from the dermal tissue in formamide for 18 hours at 60° C. Following centrifugation, the absorbance was measured at 620 nm. The amount of dermal permeability observed in saline injected animals was set at 1. Data are presented as mean±S.E. of five individual mice with each treatment in duplicate (six total injections per animal).

Biochemical assays: HMVEC were grown to confluence on fibronectin-coated dishes and starved overnight in EBM-2+0.2% BSA. The next day, the cells were stimulated with 50 ng/mL VEGF-A for 5 minutes, washed twice with ice-cold PBS and lysed in 50 mM Tris pH 7.4, 150 mM NaCl, 10 mM MgCl₂, 1 mM DTT, 10% Glycerol, 1% NP-40, 0.5% Sodium Deoxycholate, 0.1% SDS, 1× protease inhibitors, 1× phosphatase inhibitors. Lysates were combined with 2× sample buffer, separated by SDS-PAGE and probed with antibodies to phospho-VEGFR2, phospho-p42/44 and phospho-Src (Cell Signaling) at 1:1000. For Rac activation assays, crude membrane preps were generated⁹ and GTP-Rac was precipitated with 20 μg/ml GST-PBD. Following three washes with lysis buffer, bound proteins were eluted with 2× sample buffer. Rac1 was detected by western blotting with monoclonal antibodies (BD Biosciences).

ARF6 Activation Assay: Cells were detached from cell culture dishes, held in suspension for 1 h in DMEM+0.2% BSA, and plated onto bacterial Petri dishes coated with 5 μg/ml fibronectin for 5 min. The cells were then washed twice with ice-cold PBS and lysed in 50 mM Tris pH 7.0, 500 mM NaCl, 1 mM MgCl₂, 1 mM EGTA, 1 mM DTT, 0.5% NP-40, 1× protease inhibitors, 1× phosphatase inhibitors and 50 μg/ml GST-GGA3-VHS-GAT. The lysate was centrifuged for 5 min at 14,000 rpm and the supernatant was incubated with 50 μl of glutathione agarose for 30 min at 4° C. Following three washes with lysis buffer, bound proteins were eluted with 2× sample buffer. ARF6 was detected by western blotting with ARF6-specific antibodies.

Statistical Analysis: Statistical significance was determined using the Student's t-test or ANOVA, where appropriate.

### REFERENCES

Afzal A, Shaw L C, Ljubimov A V, Boulton M E, Segal M S, Grant M B. Retinal and choroidal microangiopathies: Therapeutic opportunities. Microvasc Res 2007.
Armour K L, Clark M R, Hadley A G, Williamson L M. Recombinant human IgG molecules lacking Fcgamma receptor I binding and monocyte triggering activities. Eur J Immunol 1999; 29(8):2613-24.
Balasubramanian, N., Scott, D. W., Castle, J. D., Casanova, J. E. & Schwartz, M. A. Arf6 and microtubules in adhesion-dependent trafficking of lipid rafts. Nature cell biology 9, 1381-1391 (2007).
Bashaw, G. J., Kidd, T., Murray, D., Pawson, T., and Goodman, C. S. (2000). Repulsive axon guidance: Abelson and Enabled play opposing roles downstream of the roundabout receptor. Cell 101, 703-715.
Battye, R., Stevens, A., and Jacobs, J. R. (1999). Axon repulsion from the midline of the Drosophila CNS requires slit function. Development 126, 2475-2481.
Battye R, Stevens A, Perry R L, Jacobs J R. Repellent signaling by Slit requires the leucine-rich repeats. J Neurosci 2001; 21(12):4290-8.
Bedell, V. M., Yeo, S. Y., Park, K. W., Chung. J., Seth, P., Shivalingappa, V., Zhao, J., Obara, T., Sukhatme, V. P., Drummond, I. A., Li, D. Y., and Ramchandran, R. (2005). roundabout 4 is essential for angiogenesis in vivo. Proc. Natl. Acad. Sci. U.S.A. 102, 6373-6378.
Brooks, P. C., Clark, R. A., and Cheresh, D. A. (1994). Requirement of vascular integrin alpha v beta 3 for angiogenesis. Science 264, 569-571.
Brooks, P. C., Montgomery, A. M., Rosenfeld, M., Reisfeld, R. A., Hu, T., Klier, G., and Cheresh, D. A. (1994). Integrin alpha v beta 3 antagonists promote tumor regression by inducing apoptosis of angiogenic blood vessels. Cell 30, 1157-1164.
Brose, K., Bland, K. S., Wang, K. H., ARNOtt, D., Henzel, W., Goodman, C. S., Tessier-Lavigne, M., and Kidd, T. (1999). Slit proteins bind Robo receptors and have an evolutionarily conserved role in repulsive axon guidance. Cell 19, 795-806.
Brown D M, Kaiser P K, Michels M, Soubrane G, Heier J S, Kim R Y, Sy J P, Schneider S; ANCHOR Study Group. Ranibizumab versus verteporfin for neovascular age-related macular degeneration. N Engl J Med. 2006 355(14):1432-44.
Brown, M. C., and Turner, C. E. (2004). Paxillin: adapting to change. Physiol. Rev. 84, 1315-1339.
Byzova, T. V., Goldman, C. K., Pampori, N., Thomas, K. A., Bert, A., Shattil, S. J., and Plow, E. F. (2000). A mechanism for modulation of cellular responses to VEGF: activation of the integrins. Mol. Cell. 6, 851-860.
Carmeliet P, Tessier-Lavigne M. Common mechanisms of nerve and blood vessel wiring. Nature 2005; 436(7048):193-200.
Cheng H J, Nakamoto M, Bergemann A D, Flanagan J G. Complementary gradients in expression and binding of ELF-1 and Mek4 in development of the topographic retinotectal projection map. Cell 1995; 82(3):371-81.
Chun D W, Heier J S, Topping T M, Duker J S, Bankert J M. A pilot study of multiple intravitreal injections of ranibizumab in patients with center-involving clinically significant diabetic macular edema. Opthalmology 2006; 113(10):1706-12.
Cross M J, Dixelius J, Matsumoto T, Claesson-Welsh L. VEGF-receptor signal transduction. Trends in biochemical sciences 2003; 28(9):488-94.
Culotti J G, Merz D C. DCC and netrins. Curr Opin Cell Biol 1998; 10(5):609-13.
Diabetic Retinopathy Clinical Research Network, Scott I U, Edwards A R, Beck R W, Bressler N M, Chan C K, Elman M J, Friedman S M, Greven C M, Maturi R K, Pieramici D J, Shami M, Singerman L J, Stockdale C R. A phase II randomized clinical trial of intravitreal bevacizumab for diabetic macular edema. Opthalmology. 2007 114(10):1860-7.
Dickson B J. Molecular mechanisms of axon guidance. Science 2002; 298(5600):1959-64.
Dong Q G, Bemasconi S, Lostaglio S, et al. A general strategy for isolation of endothelial cells from murine tissues. Characterization of two endothelial cell lines from the murine lung and subcutaneous sponge implants. Arterioscler Thromb Vasc Biol 1997; 17(8):1599-604.
Dorrell M, Uusitalo-Jarvinen H, Aguilar E, Friedlander M. Ocular neovascularization: basic mechanisms and therapeutic advances. Surv Opthalmol. 2007 52 Suppl 1:S3-19.
Drescher U, Kremoser C, Handwerker C, Loschinger J, Noda M, Bonhoeffer F. In vitro guidance of retinal ganglion cell axons by RAGS, a 25 kD tectal protein related to ligands for Eph receptor tyrosine kinases. Cell 1995; 82(3):359-70.
D'Souza-Schorey, C., and Chavrier, P. (2006). ARF proteins: roles in membrane traffic and beyond. Nat. Rev. Mol. Cell. Biol. 7, 347-358.
Eliceiri B P, Cheresh D A. (2000). Role of alpha v integrins during angiogenesis. Cancer J. 6, S245-249.
Eliceiri B P, Paul R, Schwartzberg P L, Hood J D, Leng J, Cheresh D A. Selective requirement for Src kinases during VEGF-induced angiogenesis and vascular permeability. Molecular cell 1999; 4(6):915-24.
Eliceiri B P, Puente X S, Hood J D, et al. Src-mediated coupling of focal adhesion kinase to integrin alpha(v) beta5 in vascular endothelial growth factor signaling. The Journal of cell biology 2002; 157(1):149-60.

Francis, S. E., Goh, K. L., Hodivala-Dilke, K., Bader, B. L., Stark, M., Davidson, D., and Hynes, R. O. (2002). Central roles of alpha5beta 1 integrin and fibronectin in vascular development in mouse embryos and embryoid bodies. Arterioscler. Thromb. Vasc. Biol. 22, 927-933.
Garrett T A, Van Buul J D, Burridge K. VEGF-induced Rac1 activation in endothelial cells is regulated by the guanine nucleotide exchange factor Vav2. Experimental cell research 2007; 313(15):3285-97.
Gavard J, Gutkind J S. VEGF controls endothelial-cell permeability by promoting the beta-arrestin-dependent endocytosis of VE-cadherin. Nature cell biology 2006; 8(11):1223-34.
Gerhardt H, Golding M, Fruttiger M, et al. VEGF guides angiogenic sprouting utilizing endothelial tip cell filopodia. The Journal of cell biology 2003; 161(6):1163-77.
Goldfinger, L. E., Han, J., Kiosses, W. B., Howe, A. K., and Ginsberg, M. H. (2003). Spatial restriction of alpha4 integrin phosphorylation regulates lamellipodial stability and alpha4beta1-dependent cell migration. J. Cell Biol. 162, 731-741.
Markus Hafner, Anton Schmitz, Imke Gru\ddot{}ne, Seergazhi G. Srivatsan, Bianca Paul, Waldemar Kolanus, Thomas Quast, Elisabeth Kremmer, Inga Bauer & Michael Famulok (2006). Inhibition of cytohesins by SecinH3 leads to hepatic insulin resistance. Nature. 444, 941-944.
Hagel, M., George, E. L., Kim, A., Tamimi, R., Opitz, S. L., Turner, C. E., Imamoto, A., and Thomas, S. M. (2002). The adaptor protein paxillin is essential for normal development in the mouse and is a critical transducer of fibronectin signaling. Mol. Cell. Biol. 22, 901-915.
Han, J., Liu, S., Rose, D. M., Schlaepfer, D. D., McDonald, H., and Ginsberg, M. H. (2001). Phosphorylation of the integrin alpha 4 cytoplasmic domain regulates paxillin binding. J. Biol. Chem. 276, 40903-40909.
Hohenester E, Hussain S, Howitt J A. Interaction of the guidance molecule Slit with cellular receptors. Biochem Soc Trans 2006; 34(Pt 3):418-21.
Hohenester, E., Hussain, S., and Howitt, J. A. (2006). Interaction of the guidance molecule Slit with cellular receptors. Biochem. Soc. Trans. 34, 418-421.
Hong K, Hinck L, Nishiyama M, Poo M M, Tessier-Lavigne M, Stein E. A ligand-gated association between cytoplasmic domains of UNC5 and DCC family receptors converts netrin-induced growth cone attraction to repulsion. Cell 1999; 97(7):927-41.
Howitt J A, Clout N J, Hohenester E. Binding site for Robo receptors revealed by dissection of the leucine-rich repeat region of Slit. Embo J 2004; 23(22):4406-12.
Hu, H., Li, M., Labrador, J. P., McEwen, J., Lai, E. C., Goodman, C. S., and Bashaw, G. J. (2005). Cross GTPase-activating protein (CrossGAP)/Vilse links the Roundabout receptor to Rac to regulate midline repulsion. Proc. Natl. Acad. Sci. U.S.A. 102, 4613-4618.
Huminiecki, L., and Bicknell, R. (2000). In silico cloning of novel endothelial specific genes. Genome Res. 10, 1796-1806.
Huminiecki L, Gorn M, Suchting S, Poulsom R, Bicknell R. Magic roundabout is a new member of the roundabout receptor family that is endothelial specific and expressed at sites of active angiogenesis. Genomics 2002; 79(4):547-52.
Huminiecki, L., Gom, M., Suchting, S., Poulsom, R., and Bicknell, R. (2002). Magic roundabout is a new member of the roundabout receptor family that is endothelial specific and expressed at sites of active angiogenesis. Genomics 79, 547-552.
Ikeda, S. et al. Novel role of ARF6 in vascular endothelial growth factor-induced signaling and angiogenesis. Circulation research 96, 467-475 (2005).
Jain R K. Molecular regulation of vessel maturation. Nat Med 2003; 9(6):685-93.
Jin, S. W., Beis, D., Mitchell, T., Chen, J. N., and Stainier, D. Y. (2005). Cellular and molecular analyses of vascular tube and lumen formation in zebrafish. Development 132, 5199-5209.
Jones C A, Li D Y. Common cues regulate neural and vascular patterning. Curr Opin Genet Dev. 2007 August; 17(4):332-6.
Kanellis, J., Garcia, G. E., Li, P., Parra, G., Wilson, C. B., Rao, Y., Han, S., Smith, C. W., Johnson, R. J., Wu, J. Y., and Feng, L. (2004). Modulation of inflammation by slit protein in vivo in experimental crescentic glomerulonephritis. Am. J. Pathol. 165, 341-352.
Katoh Y, Katoh M. Comparative genomics on SLIT1, SLIT2, and SLIT3 orthologs. Oncol Rep 2005; 14(5):1351-5.
Kaur, S., Castellone, M. D., Bedell, V. M., Konar, M., Gutkind, J. S., and Ramchandran, R. (2006). Robo4 signaling in endothelial cells implies attraction guidance mechanisms. J. Biol. Chem. 281, 11347-11356.
Kidd, T., Bland, K. S., and Goodman, C. S. (1999). Slit is the midline repellent for the robo receptor in Drosophila. Cell 96, 785-794.
Kidd, T., Brose, K., Mitchell, K. J., Fetter, R. D., Tessier-Lavigne, M., Goodman, C. S., and Tear, G. (1998). Roundabout controls axon crossing of the CNS midline and defines a novel subfamily of evolutionarily conserved guidance receptors. Cell 92, 205-215.
Kidd, T., et al. Roundabout controls axon crossing of the CNS midline and defines a novel subfamily of evolutionarily conserved guidance receptors. Cell 92, 205-215 (1998).
Kimmel, C. B., Ballard, W. W., Kimmel, S. R., Ullmann, B., and Schilling, T. F. (1995). Stages of embryonic development of the zebrafish. Dev. Dyn. 203, 253-310.
Lantry L E. Ranibizumab, a mAb against VEGF-A for the potential treatment of age-related macular degeneration and other ocular complications. Curr Opin Mol. Ther. 2007 9(6):592-602.
Lauffenburger, D. A., and Horwitz, A. F. (1996). Cell migration: a physically integrated molecular process. Cell 84, 359-369.
Lawson, N. D., and Weinstein, B. M. (2002). In vivo imaging of embryonic vascular development using transgenic zebrafish. Dev. Biol. 248, 307-318.
Li, H. S., Chen, J. H., Wu, W., Fagaly, T., Zhou, L., Yuan, W., Dupuis, S., Jiang, Z. H., Nash, W., Gick, C., Ornitz, D. M., Wu, J. Y., and Rao, Y. (1999). Vertebrate slit, a secreted ligand for the transmembrane protein roundabout, is a repellent for olfactory bulb axons. Cell 19, 807-818.
Li Q, Olsen B R. Increased angiogenic response in aortic explants of collagen XVIII/endostatin-null mice. Am J Pathol 2004; 165(2):415-24.
Lim Y C, Garcia-Cardena G, Allport J R, et al. Heterogeneity of endothelial cells from different organ sites in T-cell subset recruitment. Am J Pathol 2003; 162(5):1591-601.
Lima e Silva R, Saishin Y, Saishin Y, et al. Suppression and regression of choroidal neovascularization by polyamine analogues. Investigative opthalmology & visual science 2005; 46(9):3323-30.
Little M, Rumballe B, Georgas K, Yamada T, Teasdale R D. Conserved modularity and potential for alternate splicing in mouse and human Slit genes. Int J Dev Biol 2002; 46(4):385-91.
Liu, S., and Ginsberg, M. H. (2000). Paxillin binding to a conserved sequence motif in the alpha 4 integrin cytoplasmic domain. J. Biol. Chem. 275, 22736-22742.
Liu, S., Kiosses, W. B., Rose, D. M., Slepak, M., Salgia, R., Griffin, J. D., Turner, C. E., Schwartz, M. A., and Ginsberg, M. H. (2002). A fragment of paxillin binds the alpha 4 integrin cytoplasmic domain (tail) and selectively inhibits alpha 4-mediated cell migration. J. Biol. Chem. 277, 20887-20894.
Liu, S., Thomas, S. M., Woodside, D. G., Rose, D. M., Kiosses, W. B., Pfaff, M., and Ginsberg, M. H. (1999). Binding of paxillin to alpha4 integrins modifies integrin-dependent biological responses. Nature 402, 676-681.
Long, H., et al. Conserved roles for Slit and Robo proteins in midline commissural axon guidance. Neuron 42, 213-223 (2004).
Lundstrom, A., Gallio, M., Englund, C., Steneberg, P., Hemphala, J., Aspenstrom, P., Keleman, K., Falileeva, L., Dickson, B. J., and Samakovlis, C. (2004). Vilse, a conserved Rac/Cdc42 GAP mediating Robo repulsion in tracheal cells and axons. Genes Dev. 18, 2161-2171.
Mahabeleshwar, G. H., Feng, W., Reddy, K., Plow, E. F. & Byzova, T. V. Mechanisms of integrin-vascular endothelial growth factor receptor cross-activation in angiogenesis. Circulation research 101, 570-580 (2007).
Marillat, V., Cases, O., Nguyen-Ba-Charvet, K. T., Tessier-Lavigne, M., Sotelo, C., and Chedotal, A. (2002). Spatiotemporal expression patterns of slit and robo genes in the rat brain. J. Comp. Neurol. 442, 130-155.
Matthay M A, Zimmerman G A. (2005). Acute lung injury and the acute respiratory distress syndrome: four decades of inquiry into pathogenesis and rational management. Am J Respir Cell Mol. Biol.; 33(4):319-27.
Nakamura, K., Yano, H., Uchida, H., Hashimoto, S., Schaefer, E., and Sabe, H. (2000). Tyrosine phosphorylation of paxillin alpha is involved in temporospatial regulation of paxillin-containing focal adhesion formation and F-actin organization in motile cells. J. Biol. Chem. 275, 27155-27164.
Navankasattusas, S., K. J. Whitehead, A. Suli, L. K. Sorensen, A. H. Lim, J. Zhao, K. R. Thomas, C. B. Chien, and D. Y. Li. The netrin receptor, Unc5b, promotes angiogenesis in specific vascular beds. Development (in press).
Nishiya, N., Kiosses, W. B., Han, J., and Ginsberg, M. H. (2005). An alpha4 integrin-paxillin-Arf-GAP complex restricts Rac activation to the leading edge of migrating cells. Nat. Cell Biol. 7, 343-352.
Nobes, C. D., and Hall, A. (1995). Rho, rac, and cdc42 GTPases regulate the assembly of multimolecular focal complexes associated with actin stress fibers, lamellipodia, and filopodia. Cell 81, 53-62.
Nobes, C. D. & Hall, A. Rho GTPases control polarity, protrusion, and adhesion during cell movement. The Journal of cell biology 144, 1235-1244 (1999).
Ojima T, Takagi H, Suzuma K, Oh H, Suzuma I, Ohashi H, Watanabe D, Suganami E, Murakami T, Kurimoto M, Honda Y, Yoshimura N. EphrinA1 inhibits vascular endothelial growth factor-induced intracellular signaling and suppresses retinal neovascularization and blood-retinal barrier breakdown. Am J Pathol. 2006 January; 168(1):331-9.
Ozaki H, Seo M S, Ozaki K, et al. Blockade of vascular endothelial cell growth factor receptor signaling is sufficient to completely prevent retinal neovascularization. Am J Pathol 2000; 156(2):697-707.
Park K W, Crouse D, Lee M, et al. The axonal attractant Netrin-1 is an angiogenic factor. Proc Natl Acad Sci USA 2004; 101(46):16210-5.
Park K W, Morrison C M, Sorensen L K, et al. Robo4 is a vascular-specific receptor that inhibits endothelial migration. Dev Biol 2003; 261(1):251-67.
Park, K. W., Morrison, C. M., Sorensen, L. K., Jones, C. A., Rao, Y., Chien, C. B., Wu, J. Y., Urness, L. D., and Li, D. Y. (2003). Robo4 is a vascular-specific receptor that inhibits endothelial migration. Dev. Biol. 261, 251-267.
Raper J A. Semaphorins and their receptors in vertebrates and invertebrates. Curr Opin Neurobiol 2000; 10(1):88-94.
Reutershan J, Morris M A, Burcin T L, Smith D F, Chang D, Saprito M S, Ley K. Critical role of endothelial CXCR2 in LPS-induced neutrophil migration into the lung. J Clin Invest. 2006 116(3):695-702.
Ridley, A. J., Schwartz, M. A., Burridge, K., Firtel, R. A., Ginsberg, M. H., Borisy, G., Parsons, J. T., and Horwitz, A. R. (2003). Cell migration: integrating signals from front to back. Science. 302, 1704-1709.
Rosenfeld P J, Brown D M, Heier J S, et al. Ranibizumab for neovascular age-related macular degeneration. N Engl J Med 2006; 355(14):1419-31.
Ruhrberg C, Gerhardt H, Golding M, et al. Spatially restricted patterning cues provided by heparin-binding VEGF-A control blood vessel branching morphogenesis. Genes & development 2002; 16(20):2684-98.
Salgia, R., Li, J. L., Ewaniuk, D. S., Wang, Y. B., Sattler, M., Chen, W. C., Richards, W., Pisick, E., Shapiro, G. I., Rollins, B. J., Chen, L. B., Griffin, J. D., and Sugarbaker, D. J. (1999). Expression of the focal adhesion protein paxillin in lung cancer and its relation to cell motility. Oncogene. 18, 67-77.
Seeger, M., Tear, G., Ferres-Marco, D., and Goodman, C. S. (1993). Mutations affecting growth cone guidance in Drosophila: genes necessary for guidance toward or away from the midline. Neuron 10, 409-426.
Senger, D. R., Claffey, K. P., Benes, J. E., Perruzzi, C. A., Sergiou, A. P., and Detmar, M. (1997). Angiogenesis promoted by vascular endothelial growth factor: regulation through alpha1beta1 and alpha2beta 1 integrins. Proc. Natl. Acad. Sci. U.S.A. 94, 13612-13617.
Seth, P., Lin, Y., Hanai, J., Shivalingappa, V., Duyao, M. P., and Sukhatme, V. P. Magic roundabout, a tumor endothelial marker: expression and signaling. Biochem. Biophys. Res. Commun. 332, 533-541.
Seth P, Lin Y, Hanai J, Shivalingappa V, Duyao M P, Sukhatme V P. Magic roundabout, a tumor endothelial marker: expression and signaling. Biochem Biophys Res Commun 2005; 332(2):533-41.
Shields R L, Namenuk A K, Hong K, et al. High resolution mapping of the binding site on human IgG1 for Fc gamma RI, Fc gamma RII, Fc gamma RIII, and FcRn and design of IgG1 variants with improved binding to the Fc gamma R. J Biol Chem 2001; 276(9):6591-604.
Smith L E, Wesolowski E, McLellan A, et al. Oxygen-induced retinopathy in the mouse. Investigative opthalmology & visual science 1994; 35(1):101-11.
Soga, N., Connolly, J. O., Chellaiah, M., Kawamura, J., and Hruska, K. A. (2001). Rac regulates vascular endothelial growth factor stimulated motility. Cell Commun. Adhes. 8, 1-13.
Soga, N., Namba, N., McAllister, S., Cornelius, L., Teitelbaum, S. L., Dowdy, S. F., Kawamura, J., and Hruska, K. A. (2001). Rho family GTPases regulate VEGFstimulated endothelial cell motility. Exp. Cell Res. 269, 73-87.
Soldi, R., Mitola, S., Strasly, M., Defilippi, P., Tarone, G., and Bussolino, F. (1999). Role of alphavbeta3 integrin in the activation of vascular endothelial growth factor receptor-2. EMBO J. 18, 882-892.
Stein E, Tessier-Lavigne M. Hierarchical organization of guidance receptors: silencing of netrin attraction by slit through a Robo/DCC receptor complex. Science 2001; 291(5510):1928-38.
Suchting, S., Heal, P., Tahtis, K., Stewart, L. M., and Bicknell, R. (2005). Soluble Robo4 receptor inhibits in vivo angiogenesis and endothelial cell migration. FASEB J. 19, 121-138.
Turner, C. E. (2000). Paxillin interactions. J. Cell Sci. 113, 139-140. Wang, K. H., Brose, K., ARNOtt, D., Kidd, T., Goodman, C. S., Henzel, W., and Tessier-Lavigne, M. (1999). Biochemical purification of a mammalian slit protein as a positive regulator of sensory axon elongation and branching. Cell 96, 771-784.
Turner, C. E. Paxillin and focal adhesion signalling. Nature cell biology 2, E231-236 (2000).
Uemura A, Kusuhara S, Katsuta H, Nishikawa S. Angiogenesis in the mouse retina: a model system for experimental manipulation. Experimental cell research 2006; 312(5):676-83.
Umess L D, Li D Y. Wiring the vascular circuitry: from growth factors to guidance cues. Curr Top Dev Biol 2004; 62:87-126.
Wang, B., Xiao, Y., Ding, B. B., Zhang, N., Yuan, X., Gui, L., Qian, K. X., Duan, S., Chen, Z., Rao, Y., and Geng, J. G. (2003). Induction of tumor angiogenesis by Slit-Robo signaling and inhibition of cancer growth by blocking Robo activity. Cancer Cell 4, 19-29.
Wang, K. H. et al. Biochemical purification of a mammalian slit protein as a positive regulator of sensory axon elongation and branching. Cell 96, 771-784 (1999).
Watanabe D, Suzuma K, Matsui S, Kurimoto M, Kiryu J, Kita M, Suzuma I, Ohashi H, Ojima T, Murakami T, Kobayashi T, Masuda S, Nagao M, Yoshimura N, Takagi H. Erythropoietin as a retinal angiogenic factor in proliferative diabetic retinopathy. N Engl J. Med. 2005 353(8):782-92.
Weinstein, B. M. (2002). Plumbing the mysteries of vascular development using the zebrafish. Semin. Cell Dev. Biol. 13, 515-522.
Werdich X Q, McCollum G W, Rajaratnam V S, Penn J S. Variable oxygen and retinal VEGF levels: correlation with incidence and severity of pathology in a rat model of oxygen-induced retinopathy. Exp Eye Res 2004; 79(5):623-30.
West, K. A., Zhang, H., Brown, M. C., Nikolopoulos, S. N., Riedy, M. C., Horwitz, A. F., and Turner, C. E. (2001). The LD4 motif of paxillin regulates cell spreading and motility through an interaction with paxillin kinase linker (PKL). J. Cell Biol. 154, 161-176.
Westerfield, M. (2000). The zebrafish book. A guide for the laboratory use of zebrafish (Danio rerio). 4th ed. (Univ. of Oregon Press, Eugene).
Wilkinson D G, Bhatt S, Herrmann B G. Expression pattern of the mouse T gene and its role in mesoderm formation. Nature 1990; 343(6259):657-9.
Wilson B D, Ii M, Park K W, et al. Netrins promote developmental and therapeutic angiogenesis. Science 2006; 313(5787):640-4.
Wojciak-Stothard, B., Potempa, S., Eichholtz, T., and Ridley, A. J. (2001). Rho and Rac but not Cdc42 regulate endothelial cell permeability. J. Cell Sci. 114, 1343-1355.
Wong, K., Ren, X. R., Huang, Y. Z., Xie, Y., Liu, G., Saito, H., Tang, H., Wen, L., Brady-Kalnay, S. M., Mei, L., Wu, J. Y., Xiong, W. C., and Rao, Y. (2001). Signal transduction in neuronal migration: roles of GTPase activating proteins and the small GTPase Cdc42 in the Slit-Robo pathway. Cell 107, 209-221.
Wu, J. Y., Feng, L., Park, H. T., Havlioglu, N., Wen, L., Tang, H., Bacon, K. B., Jiang, Z., Zhang, X., and Rao, Y. (2001). The neuronal repellent Slit inhibits leukocyte chemotaxis induced by chemotactic factors. Nature 410, 948-952.
Wu, W., Wong, K., Chen, J., Jiang, Z., Dupuis, S., Wu, J. Y., and Rao, Y. (1999). Directional guidance of neuronal migration in the olfactory system by the protein Slit. Nature 400, 331-336.
Xu Q, Qaum T, Adamis A P. Sensitive blood-retinal barrier breakdown Quantitation using evans blue. Invest Opthalmol Vis Sci. 2001; 42(3):789-94.
Yano, H., Mazaki, Y., Kurokawa, K., Hanks, S. K., Matsuda, M., and Sabe, H. (2004). Roles played by a subset of integrin signaling molecules in cadherin based cell-cell adhesion. J. Cell Biol. 166, 283-295.
Yano, H., Uchida, H., Iwasaki, T., Mukai, M., Akedo, H., Nakamura, K., Hashimoto, S., and Sabe, H. (2000). Paxillin alpha and Crk-associated substrate exert opposing effects on cell migration and contact inhibition of growth through tyrosine phosphorylation. Proc. Natl. Acad. Sci. U.S.A. 97, 9076-9081.
Yu, T. W., Hao, J. C., Lim, W., Tessier-Lavigne, M., and Bargmann, C. I. (2002). Shared receptors in axon guidance: SAX-3/Robo signals via UNC-34/Enabled and a Netrin-independent UNC-40/DCC function. Nat. Neurosci. 5, 1147-1154.
Yuan, W., Zhou, L., Chen, J. H., Wu, J. Y., Rao, Y., and Ornitz, D. M. (1999). The mouse SLIT family: secreted ligands for ROBO expressed in patterns that suggest a role in morphogenesis and axon guidance. Dev. Biol. 212, 290-306.
Yuminamochi, T., Yatomi, Y., Osada, M., Ohmori, T., Ishii, Y., Nakazawa, K., Hosogaya, S., and Ozaki, Y. (2003). Expression of the LIM proteins paxillin and Hic-5 in human tissues. J. Histochem. Cytochem. 51, 513-521.
Zallen, J. A., Yi, B. A., and Bargmann, C. I. (1998). The conserved immunoglobulin superfamily member SAX-3/Robo directs multiple aspects of axon guidance in C. elegans. Cell 92, 217-227.
Zhang, Q. et al. Small-molecule synergist of the Wnt/beta-catenin signaling pathway. Proceedings of the National Academy of Sciences of the United States of America 104, 7444-7448 (2007).
Zhu, Y., Li, H., Zhou, L., Wu, J. Y., and Rao, Y. (1999). Cellular and molecular guidance of GABAergic neuronal migration from an extracortical origin to the neocortex. Neuron 23, 473-485.

1. A composition comprising a ligand of a Robo4 receptor that interacts with the Robo4 receptor in a manner that results in one or more of inhibition of Rac, inhibition of ARF6, preservation of endothelial barrier function, blocking of VEGF signaling downstream of the VEGF receptor, inhibition of vascular leak, inhibition of pathologic angiogenesis, and signal inhibition of multiple angiogenic, permeability and inflammatory factors, wherein the ligand comprises a Slit ligand.

2. The composition of claim 1 wherein the Slit ligand comprises a ligand selected from one or more of a Slit1 ligand, a Slit2 ligand and a Slit3 ligand.

3. A composition according to claim 1, wherein the Slit ligand comprises a ligand selected from one or more of Slit1 (SEQ ID NO: 1), Slit2 (SEQ ID NO: 2), Slit3 (SEQ ID NO: 3), and polypeptides comprising an amino acid sequence having at least about 70%, at least about 75%, at least about 80%, at least about 85%, at least about 90%, at least about 95%, or at least about 100% sequence identity to one of SEQ ID NO: 1, SEQ ID NO: 2, SEQ ID NO: 3.

4. A composition according to claim 1, wherein the Slit ligand comprises a Slit2 ligand selected from one or more of Slit2N (SEQ ID NO: 7), SEQ ID NO: 8, Slit2AP (SEQ ID NO: 9), Slit2 D1 (SEQ ID NO: 10), Slit2 D1-D2 (SEQ ID NO: 11), Slit2 D1-D3 (SEQ ID NO: 12), Slit2 D1-D4 (SEQ ID NO: 13), Slit2 D1-E5 (SEQ ID NO: 14), Slit2 D1-E6 (SEQ ID NO: 15), and polypeptides comprising an amino acid sequence having at least about 70%, at least about 75%, at least about 80%, at least about 85%, at least about 90%, at least about 95%, or at least about 100% sequence identity to any one of SEQ ID NO: 7 through SEQ ID NO: 15.

5. A composition according to claim 1, wherein the Slit ligand comprises a ligand selected from amino acids 1-1132 of Slit1 (SEQ ID NO: 4), amino acids 1-1119 of Slit2 (SEQ ID NO: 5), amino acids 1-1118 of Slit3 (SEQ ID NO: 6), amino acids 281-511 of Slit1 (SEQ ID NO: 16), amino acids 271-504 of Slit2 (SEQ ID NO: 17), and polypeptides comprising an amino acid sequence having at least about 70%, at least about 75%, at least about 80%, at least about 85%, at least about 90%, at least about 95%, or at least about 100% sequence identity to any one of SEQ ID NO: 4 through SEQ ID NO: 6, SEQ ID NO: 16, and SEQ ID NO: 17.

6. A composition according to claim 1, wherein the composition is prepared as a pharmaceutical composition for the treatment of vascular permeability associated with a disease state selected from infectious and non-infectious diseases that may result in a cytokine storm, graft versus host disease (GVHD), adult respiratory distress syndrome (ARDS), sepsis, avian influenza, smallpox, and systemic inflammatory response syndrome (SIRS), ischemia/reperfusion injury following stroke or myocardial infarction, edema associated with brain tumors, ascites associated with malignancies, Meigs' syndrome, lung inflammation, nephrotic syndrome, pericardial effusion and pleural effusion, inflammation, allergic diseases, cancer, cerebral stroke, myocardial infarction, pulmonary and cardiac insufficiency, renal failure, and retinopathies.

7. A composition according to claim 1, wherein the composition is prepared as a pharmaceutical composition for the treatment of pathologic angiogenesis associated with a disease state selected from hemangioma, solid tumors, leukemia, metastasis, telangiectasia psoriasis scleroderma, pyogenic granuloma, myocardial angiogenesis, plaque neovascularization, coronary collaterals, ischemic limb angiogenesis, corneal diseases, rubeosis, neovascular glaucoma, diabetic retinopathy (DR), retrolental fibroplasia, non-proliferative diabetic macular edema (DME), arthritis, diabetic neovascularization, age-related macular degeneration (AMD), retinopathy of prematurity (ROP), ischemic retinal vein occlusion (IRVO), wound healing, peptic ulcer, fractures, keloids, vasculogenesis, hematopoiesis, ovulation, menstruation, and placentation.

8. A composition comprising a small molecule that inhibits the availability, activation or activity of an ARF-GEF in a manner that results in one or more of inhibition of Rac, inhibition of ARF6, preservation of endothelial barrier function, blocking of VEGF signaling downstream of the VEGF receptor, inhibition of vascular leak, inhibition of pathologic angiogenesis, and signal inhibition of multiple angiogenic, permeability and inflammatory factors.

9. A composition according to claim 8, wherein the small molecule inhibits the availability, activation or activity of a cytohesin.

10. A composition according to claim 8, wherein the small molecule inhibits the availability, activation or activity of a cytohesin selected from ARNO and the ARNO family of cytohesins.

11. A composition according to claim 8, wherein the small molecule is selected from one or more compounds having the following chemical formula (Formula 1): 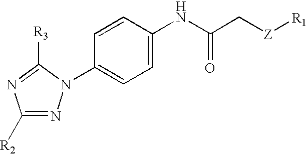
wherein:
R¹ and R³ are independently chosen from optionally substituted aryl, optionally substituted heteroaryl, optionally substituted cycloalkyl, or optionally substituted heterocycle;
R² is chosen from hydrogen, lower alkoxy, lower alkyl, halogen or hydroxy;
Z is chosen from O, S, NH, alkylene or a single bond; or
pharmaceutically acceptable salts, solvates or hydrates thereof.

12. A composition according to claim 11, wherein R³ of at least one of the one or more compounds is substituted with 1 to 5 substituents independently chosen from halogen, lower alkyl, lower alkoxy, heteroatom lower alkyl, hydroxy, or methylene dioxy, wherein two substituents together may form a fused cycloalkyl or heterocyclic ring structure.

13. A composition according to claim 11, wherein of at least one of the one or more compounds comprises: R¹ chosen from unsubstituted aryl or unsubstituted heteroaryl; R² chosen from hydrogen, lower alkoxy, or lower alkyl; R³ chosen from aryl, optionally substituted with 1 to 5 substituents independently chosen from halogen, lower alkyl, lower alkoxy, or methylene dioxy, wherein two substituents together may form a fused cycloalkyl or heterocyclic ring structure; and Z chosen from O, S, or a single bond.

14. A composition according to any of claims 8 through claim 10, wherein the small molecule is selected from one or more compounds having the following chemical formula (Formula 2): 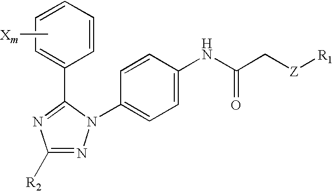
wherein:
R¹ is chosen from optionally substituted aryl, optionally substituted heteroaryl, optionally substituted cycloalkyl, or optionally substituted heterocycle;
R² is chosen from hydrogen, lower alkoxy, lower alkyl, halogen or hydroxy;
Z is chosen from O, S, NH, alkylene or a single bond;
X is independently chosen from halogen, lower alkyl, lower alkoxy, heteroatom lower alkyl, hydroxy, or methylene dioxy, wherein two substituents together may form a fused cycloalkyl or heterocyclic ring structure;
m is 0 to 5; or
pharmaceutically acceptable salts, solvates or hydrates thereof.

15. A composition according to claim 8, wherein the one or more compounds are selected from the following compounds: 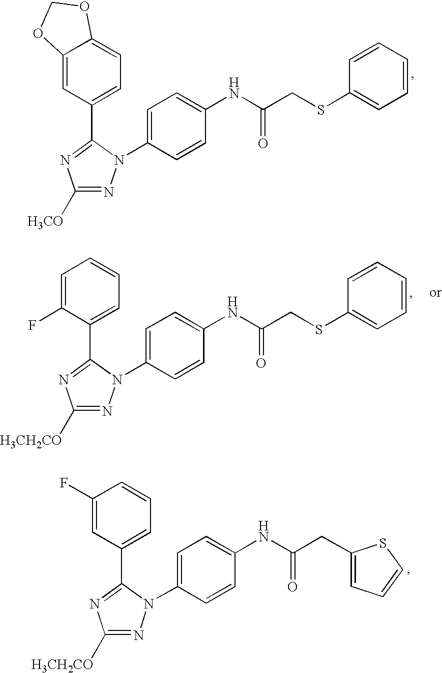
or pharmaceutically acceptable salts, solvates or hydrates thereof.

16. A composition according to claim 8, wherein the composition is prepared as a pharmaceutical composition for the treatment of vascular permeability associated with a disease state selected from infectious and non-infectious diseases that may result in a cytokine storm, graft versus host disease (GVHD), adult respiratory distress syndrome (ARDS), sepsis, avian influenza, smallpox, and systemic inflammatory response syndrome (SIRS), ischemia/reperfusion injury following stroke or myocardial infarction, edema associated with brain tumors, ascites associated with malignancies, Meigs' syndrome, lung inflammation, nephrotic syndrome, pericardial effusion and pleural effusion, inflammation, allergic diseases, cancer, cerebral stroke, myocardial infarction, pulmonary and cardiac insufficiency, renal failure, and retinopathies.

17. A composition according to claim 8, wherein the composition is prepared as a pharmaceutical composition for the treatment of pathologic angiogenesis associated with a disease state selected from hemangioma, solid tumors, leukemia, metastasis, telangiectasia psoriasis scleroderma, pyogenic granuloma, myocardial angiogenesis, plaque neovascularization, coronary collaterals, ischemic limb angiogenesis, corneal diseases, rubeosis, neovascular glaucoma, diabetic retinopathy (DR), retrolental fibroplasia, non-proliferative diabetic macular edema (DME), arthritis, diabetic neovascularization, age-related macular degeneration (AMD), retinopathy of prematurity (ROP), ischemic retinal vein occlusion (IRVO), wound healing, peptic ulcer, fractures, keloids, vasculogenesis, hematopoiesis, ovulation, menstruation, and placentation.

18. A method of inhibiting vascular permeability, comprising administering to a subject a therapeutically effective amount of a composition selected from a composition that inhibits the availability, activation or activity of one or more ARF-GEFs and a composition that promotes the availability, activation or activity of one or more ARF-GAPs.

19. A method of inhibiting pathologic angiogenesis, comprising administering to a subject a therapeutically effective amount of a composition selected from a composition that inhibits the availability, activation or activity of one or more ARF-GEFs and a composition that promotes the availability, activation or activity of one or more ARF-GAPs.

20.-30. (canceled)

31. A method according to claim 18 or 19, wherein a composition that inhibits the availability, activation or activity of one or more ARF-GEFs, comprises one or more compounds having the following chemical formula (Formula 1): 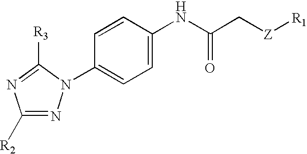
wherein:
R¹ and R³ are independently chosen from optionally substituted aryl, optionally substituted heteroaryl, optionally substituted cycloalkyl, or optionally substituted heterocycle;
R² is chosen from hydrogen, lower alkoxy, lower alkyl, halogen or hydroxy;
Z is chosen from O, S, NH, alkylene or a single bond; or
pharmaceutically acceptable salts, solvates or hydrates thereof.

32. A method according to claim 31, wherein R³ of at least one of the one or more compounds included in the composition administered to the subject is substituted with 1 to 5 substituents independently chosen from halogen, lower alkyl, lower alkoxy, heteroatom lower alkyl, hydroxy, or methylene dioxy, wherein two substituents together may form a fused cycloalkyl or heterocyclic ring structure.

33. A method according to claim 31, wherein of at least one of the one or more compounds included in the composition administered to the subject comprises: R¹ chosen from unsubstituted aryl or unsubstituted heteroaryl; R² chosen from hydrogen, lower alkoxy, or lower alkyl; R³ chosen from aryl, optionally substituted with 1 to 5 substituents independently chosen from halogen, lower alkyl, lower alkoxy, or methylene dioxy, wherein two substituents together may form a fused cycloalkyl or heterocyclic ring structure; and Z chosen from O, S, or a single bond.

34. A method according to claim 18 or 19, wherein a composition that inhibits the availability, activation or activity of one or more ARF-GEFs, comprises one or more compounds having the following chemical formula (Formula 2): 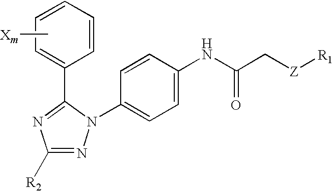
wherein:
R¹ is chosen from optionally substituted aryl, optionally substituted heteroaryl, optionally substituted cycloalkyl, or optionally substituted heterocycle;
R² is chosen from hydrogen, lower alkoxy, lower alkyl, halogen or hydroxy;
Z is chosen from O, S, NH, alkylene or a single bond;
X is independently chosen from halogen, lower alkyl, lower alkoxy, heteroatom lower alkyl, hydroxy, or methylene dioxy, wherein two substituents together may form a fused cycloalkyl or heterocyclic ring structure;
m is 0 to 5; or
pharmaceutically acceptable salts, solvates or hydrates thereof.

35. A method according to claims 31 and 34, wherein of at least one of the one or more compounds included in the composition administered to the subject comprises a compound selected from the following compounds: 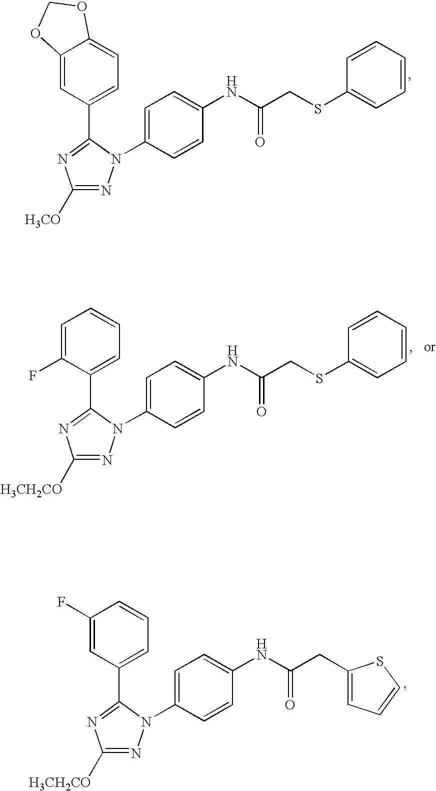
or pharmaceutically acceptable salts, solvates or hydrates thereof.

36.-44. (canceled)


SEQUENCE LISTING (17 sequences)

SEQ ID NO: 1 - protein, 1534 aa, Homo sapiens
MALTPGWGSSAGPVRPELWLLLWAAAWRLGASACPALCTCTGTTVDCHGTGLQAIPKNIPRNTERLELNGNNITRIHKNDFAGLKQLRVLQLMENQIGAVERGAFDDMKELERLRLNRNQLHMLPELLFQNNQALSRLDLSENAIQAIPRKAFRGATDLKNLQLDKNQISCIEEGAFRALRGLEVLTLNNNNITTIPVSSFNHMPKLRTFRLHSNHLFCDCHLAWLSQWLRQRPTIGLFTQCSGPASLRGLNVAEVQKSEFSCSGQGEAGRVPTCTLSSGSCPAMCTCSNGIVDCRGKGLTAIPANLPETMTEIRLELNGIKSIPPGAFSPYRKLRRIDLSNNQIAEIAPDAFQGLRSLNSLVLYGNKITDLPRGVFGGLYTLQLLLLNANKINCIRPDAFQDLQNLSLLSLYDNKIQSLAKGTFTSLRAIQTLHLAQNPFICDCNLKWLADFLRTNPIETSGARCASPRRLANKRIGQIKSKKFRCSAKEQYFIPGTEDYQLNSECNSDVVCPHKCRCEANVVECSSLKLTKIPERIPQSTAELRLNNNEISILEATGMFKKLTHLKKINLSNNKVSEIEDGAFEGAASVSELHLTANQLESIRSGMFRGLDGLRTLMLRNNRISCIHNDSFTGLRNVRLLSLYDNQITTVSPGAFDTLQSLSTLNLLANPFNCNCQLAWLGGWLRKRKIVTGNPRCQNPDFLRQIPLQDVAFPDFRCEEGQEEGGCLPRPQCPQECACLDTVVRCSNKHLRALPKGIPKNVTELYLDGNQFTLVPGQLSTFKYLQLVDLSNNKISSLSNSSFTNMSQLTTLILSYNALQCIPPLAFQGLRSLRLLSLHGNDISTLQEGIFADVTSLSHLAIGANPLYCDCHLRWLSSWVKTGYKEPGIARCAGPQDMEGKLLLTTPAKKFECQGPPTLAVQAKCDLCLSSPCQNQGTCHNDPLEVYRCACPSGYKGRDCEVSLNSCSSGPCENGGTCHAQEGEDAPFTCSCPTGFEGPTCGVNTDDCVDHACANGGVCVDGVGNYTCQCPLQYEGKACEQLVDLCSPDLNPCQHEAQCVGTPDGPRCECMPGYAGDNCSENQDDCRDHRCQNGAQCMDEVNSYSCLCAEGYSGQLCEIPPHLPAPKSPCEGTECQNGANCVDQGNRPVCQCLPGFGGPECEKLLSVNFVDRDTYLQFTDLQNWPRANITLQVSTAEDNGILLYNGDNDHIAVELYQGHVRVSYDPGSYPSSAIYSAETINDGQFHTVELVAFDQMVNLSIDGGSPMTMDNFGKHYTLNSEAPLYVGGMPVDVNSAAFRLWQILNGTGFHGCIRNLYINNELQDFTKTQMKPGVVPGCEPCRKLYCLHGICQPNATPGPMCHCEAGWVGLHCDQPADGPCHGHKCVHGQCVPLDALSYSCQCQDGYSGALCNQAGALAEPCRGLQCLHGHCQASGTKGAHCVCDPGFSGELCEQESECRGDPVRDFHQVQRGYAICQTTRPLSWVECRGSCPGQGCCQGLRLKRRKFTFECSDGTSFAEEVEKPTKCGCALCA

SEQ ID NO: 2 - protein, 1529 aa, Homo sapiens
MRGVGWQMLSLSLGLVLAILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRTSPCDNFDCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSCDREISCRGERIRDYYQKQQGYAACQTTKKVSRLECRGGCAGGQCCGPLRSKRRKYSFECTDGSSFVDEVEKVVKCGCTRCVS

SEQ ID NO: 3 - protein, 1523 aa, Homo sapiens
MAPGWAGVGAAVRARLALALALASVLSGPPAVACPTKCTCSAASVDCHGLGLRAVPRGIPRNAERLDLDRNNITRITKMDFAGLKNLRVLHLEDNQVSVIERGAFQDLKQLERLRLNKNKLQVLPELLFQSTPKLTRLDLSENQIQGIPRKAFRGITDVKNLQLDNNHISCIEDGAFRALRDLEILTLNNNNISRILVTSFNHMPKIRTLRLHSNHLYCDCHLAWLSDWLRQRRTVGQFTLCMAPVHLRGFNVADVQKKEYVCPAPHSEPPSCNANSISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSGSEDYRSRFSSECFMDLVCPEKCRCEGTIVDCSNQKLVRIPSHLPEYVTDLRLNDNEVSVLEATGIFKKLPNLRKINLSNNKIKEVREGAFDGAASVQELMLTGNQLETVHGRVFRGLSGLKTLMLRSNLIGCVSNDTFAGLSSVRLLSLYDNRITTITPGAFTTLVSLSTINLLSNPFNCNCHLAWLGKWLRKRRIVSGNPRCQKPFFLKEIPIQDVAIQDFTCDGNEESSCQLSPRCPEQCTCMETVVRCSNKGLRALPRGMPKDVTELYLEGNHLTAVPRELSALRHLTLIDLSNNSISMLTNYTFSNMSHLSTLILSYNRLRCIPVHAFNGLRSLRVLTLHGNDISSVPEGSFNDLTSLSHLALGTNPLHCDCSLRWLSEWVKAGYKEPGIARCSSPEPMADRLLLTTPTHRFQCKGPVDINIVAKCNACLSSPCKNNGTCTQDPVELYRCACPYSYKGKDCTVPINTCIQNPCQHGGTCHLSDSHKDGFSCSCPLGFEGQRCEINPDDCEDNDCENNATCVDGINNYVCICPPNYTGELCDEVIDHCVPELNLCQHEAKCIPLDKGFSCECVPGYSGKLCETDNDDCVAHKCRHGAQCVDTINGYTCTCPQGFSGPFCEHPPPMVLLQTSPCDQYECQNGAQCIVVQQEPTCRCPPGFAGPRCEKLITVNFVGKDSYVELASAKVRPQANISLQVATDKDNGILLYKGDNDPLALELYQGHVRLVYDSLSSPPTTVYSVETVNDGQFHSVELVTLNQTLNLVVDKGTPKSLGKLQKQPAVGINSPLYLGGIPTSTGLSALRQGTDRPLGGFHGCIHEVRINNELQDFKALPPQSLGVSPGCKSCTVCKHGLCRSVEKDSVVCECRPGWTGPLCDQEARDPCLGHRCHHGKCVATGTSYMCKCAEGYGGDLCDNKNDSANACSAFKCHHGQCHISDQGEPYCLCQPGFSGEHCQQENPCLGQVVREVIRRQKGYASCATASKVPIMECRGGCGPQCCQPTRSKRRKYVFQCTDGSSFVEEVERHLECGCLACS

SEQ ID NO: 4 - protein, 1132 aa, Homo sapiens
MALTPGWGSSAGPVRPELWLLLWAAAWRLGASACPALCTCTGTTVDCHGTGLQAIPKNIPRNTERLELNGNNITRIHKNDFAGLKQLRVLQLMENQIGAVERGAFDDMKELERLRLNRNQLHMLPELLFQNNQALSRLDLSENAIQAIPRKAFRGATDLKNLRLDKNQISCIEEGAFRALRGLEVLTLNNNNITTIPVSSFNHMPKLRTFRLHSNHLFCDCHLAWLSQWLRQRPTIGLFTQCSGPASLRGLNVAEVQKSEFSCSGQGEAGRVPTCTLSSGSCPAMCTCSNGIVDCRGKGLTAIPANLPETMTEIRLELNGIKSIPPGAFSPYRKLRRIDLSNNQIAEIAPDAFQGLRSLNSLVLYGNKITDLPRGVFGGLYTLQLLLLNANKINCIRPDAFQDLQNLSLLSLYDNKIQSLAKGTFTSLRAIQTLHLAQNPFICDCNLKWLADFLRTNPIETSGARCASPRRLANKRIGQIKSKKFRCSAKEQYFIPGTEDYQLNSECNSDVVCPHKCRCEANVVECSSLKLTKIPERIPQSTAELRLNNNEISILEATGMFKKLTHLKKINLSNNKVSEIEDGAFEGAASVSELHLTANQLESIRSGMFRGLDGLRTLMLRNNRISCIHNDSFTGLRNVRLLSLYDNQITTVSPGAFDTLQSLSTLNLLANPFNCNCQLAWLGGWLRKRKIVTGNPRCQNPDFLRQIPLQDVAFPDFRCEEGQEEGGCLPRPQCPQECACLDTVVRCSNKHLRALPKGIPKNVTELYLDGNQFTLVPGQLSTFKYLQLVDLSNNKISSLSNSSFTNMSQLTTLILSYNALQCIPPLAFQGLRSLRLLSLHGNDISTLQEGIFADVTSLSHLAIGANPLYCDCHLRWLSSWVKTGYKEPGIARCAGPQDMEGKLLLTTPAKKFECQGPPTLAVQAKCDLCLSSPCQNQGTCHNDPLEVYRCACPSGYKGRDCEVSLNSCSSGPCENGGTCHAQEGEDAPFTCSCPTGFEGPTCGVNTDDCVDHACANGGVCVDGVGNYTCQCPLQYEGKACEQLVDLCSPDLNPCQHEAQCVGTPDGPRCECMPGYAGDNCSENQDDCRDHRCQNGAQCMDEVNSYSCLCAEGYSGQLCEIPPHLPAPKSPCE

SEQ ID NO: 5 - protein, 1119 aa, Homo sapiens
MRGVGWQMLSLSLGLVLAILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLP

SEQ ID NO: 6 - protein, 1118 aa, Homo sapiens
MAPGWAGVGAAVRARLALALALASVLSGPPAVACPTKCTCSAASVDCHGLGLRAVPRGIPRNAERLDLDRNNITRITKMDFAGLKNLRVLHLEDNQVSVIERGAFQDLKQLERLRLNKNKLQVLPELLFQSTPKLTRLDLSENQIQGIPRKAFRGITDVKNLQLDNNHISCIEDGAFRALRDLEILTLNNNNISRILVTSFNHMPKIRTLRLHSNHLYCDCHLAWLSDWLRQRRTVGQFTLCMAPVHLRGFNVADVQKKEYVCPAPHSEPPSCNANSISCPSPCTCSNNIVDCRGKGLMEIPANLPEGIVEIRLEQNSIKAIPAGAFTQYKKLKRIDISKNQISDIAPDAFQGLKSLTSLVLYGNKITEIAKGLFDGLVSLQLLLLNANKINCLRVNTFQDLQNLNLLSLYDNKLQTISKGLFAPLQSIQTLHLAQNPFVCDCHLKWLADYLQDNPIETSGARCSSPRRLANKRISQIKSKKFRCSGSEDYRSRFSSECFMDLVCPEKCRCEGTIVDCSNQKLVRIPSHLPEYVTDLRLNDNEVSVLEATGIFKKLPNLRKINLSNNKIKEVREGAFDGAASVQELMLTGNQLETVHGRVFRGLSGLKTLMLRSNLIGCVSNDTFAGLSSVRLLSLYDNRITTITPGAFTTLVSLSTINLLSNPFNCNCHLAWLGKWLRKRRIVSGNPRCQKPFFLKEIPIQDVAIQDFTCDGNEESSCQLSPRCPEQCTCMETVVRCSNKGLRALPRGMPKDVTELYLEGNHLTAVPRELSALRHLTLIDLSNNSISMLTNYTFSNMSHLSTLILSYNRLRCIPVHAFNGLRSLRVLTLHGNDISSVPEGSFNDLTSLSHLALGTNPLHCDCSLRWLSEWVKAGYKEPGIARCSSPEPMADRLLLTTPTHRFQCKGPVDINIVAKCNACLSSPCKNNGTCTQDPVELYRCACPYSYKGKDCTVPINTCIQNPCQHGGTCHLSDSHKDGFSCSCPLGFEGQRCEINPDDCEDNDCENNATCVDGINNYVCICPPNYTGELCDEVIDHCVPELNLCQHEAKCIPLDKGFSCECVPGYSGKLCETDNDDCVAHKCRHGAQCVDTINGYTCTCPQGFSGPFCEHPPPMVLL

SEQ ID NO: 7 - protein, 1091 aa, Homo sapiens
QACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPP

SEQ ID NO: 8 - protein, 1506 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRTSPCDNFDCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSCDREISCRGERIRDYYQKQQGYAACQTTKKVSRLECRGGCAGGQCCGPLRSKRRKYSFECTDGSSFVDEVEKVVKCGC

SEQ ID NO: 9 - protein, 1498 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSCDREISCRGERIRDYYQKQQGYAACQTTKKVSRLECRGGCAGGQCCGPLRSKRRKYSFECTDGSSFVDEVEKVVKCGC

SEQ ID NO: 10 - protein, 244 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQS

SEQ ID NO: 11 - protein, 464 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKE

SEQ ID NO: 12 - protein, 687 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDV

SEQ ID NO: 13 - protein, 885 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSK

SEQ ID NO: 14 - protein, 1089 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYS

SEQ ID NO: 15 - protein, 1163 aa, Homo sapiens
ILNKVAPQACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMVLPRTSPCDNFDCQNGAQCIVRINEPICQCLPGYQGEKCEKLVSVNFINKESYLQIPSAKVRPQ

SEQ ID NO: 16 - protein, 231 aa, Homo sapiens
SCPAMCTCSNGIVDCRGKGLTAIPANLPETMTEIRLELNGIKSIPPGAFSPYRKLRRIDLSNNQIAEIAPDAFQGLRSLNSLVLYGNKITDLPRGVFGGLYTLQLLLLNANKINCIRPDAFQDLQNLSLLSLYDNKIQSLAKGTFTSLRAIQTLHLAQNPFICDCNLKWLADFLRTNPIETSGARCASPRRLANKRIGQIKSKKFRCSAKEQYFIPGTEDYQLNSECNSDV

SEQ ID NO: 17 - protein, 234 aa, Homo sapiens
LHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADL